(19) United States
(12) Patent Application Publication
(10) Pub. No.: US 2016/0002326 A1
(43) Pub. Date: Jan. 7, 2016

(54) COMPOSITIONS AND METHODS FOR TREATING RHEUMATOID ARTHRITIS

(71) Applicant: AbbVie Inc., North Chicago, IL (US)

(72) Inventors: Carolyn Cuff, Grafton, MA (US); Robert Caldwell, Franklin, WI (US); Renee Heuser, Lindenhurst, IL (US); Heikki Mansikka, Lake Forest, IL (US); Robert J. Padley, Lake Bluff, IL (US)

(21) Appl. No.: 14/736,095

(22) Filed: Jun. 10, 2015

Related U.S. Application Data
(60) Provisional application No. 62/010,430, filed on Jun. 10, 2014.
(60) Provisional application No. 62/130,362, filed on Mar. 9, 2015.

(51) Int. Cl. C07K 16/24

(52) U.S. Cl. CPC C07K 16/244; C07K 16/241; C07K 2317/565; C07K 2317/31; C07K 2317/76; A61K 2039/505

(57) ABSTRACT

The treatment of rheumatoid arthritis using engineered multivalent and multispecific binding proteins that target TNF and IL-17 is provided.

### RELATED APPLICATIONS

This application claims priority to U.S. provisional application Ser. No. 62/130,362, filed Mar. 9, 2015, and U.S. provisional application Ser. No. 62/010,430, filed Jun. 10, 2014, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 30, 2015, is named 569548_BBI-982PC_SL.txt and is 50,404 bytes in size.

### FIELD OF THE INVENTION

The present invention relates to bispecific TNF and IL-17 binding proteins, and to their uses in the prevention and/or treatment of acute and chronic immunological diseases such as rheumatoid arthritis.

### BACKGROUND OF THE INVENTION

Rheumatoid arthritis (RA) is an autoimmune disease that affects over a million Americans, with a significantly higher occurrence among women than men. Disease-modifying anti-rheumatic drugs (DMARDs) are often used to control the progression of RA and to try to prevent joint deterioration and disability. However, DMARD resistance occurs in some patients, for example, those who are receiving the DMARD methotrexate.

In addition to traditional DMARD therapies, a number of biologic therapies that target pro-inflammatory mediators, such as tumor necrosis factor-α, have been used successfully. Tumor necrosis factor-α (TNF-α) is a multifunctional pro-inflammatory cytokine secreted predominantly by monocytes and macrophages that plays a role in lipid metabolism, coagulation, insulin resistance, and endothelial function. TNF-α triggers pro-inflammatory pathways that result in tissue injury, such as degradation of cartilage and bone, induction of adhesion molecules, induction of pro-coagulant activity on vascular endothelial cells, an increase in the adherence of neutrophils and lymphocytes, and stimulation of the release of platelet activating factor from macrophages, neutrophils and vascular endothelial cells. Since TNF-α contributes to the etiology of many inflammatory disorders, including RA, it is a useful target for specific immunotherapy.

Adalimumab (also known by its trademark HUMIRA®) is a recombinant human monoclonal antibody specific for TNF-α. This monoclonal antibody binds to TNF-α and blocks its interaction with the p55 and p75 cell-surface TNF-α receptors. Adalimumab is used to treat a number of inflammatory disorders such as rheumatoid arthritis. Although Adalimumab and other TNF-α inhibitors have revolutionized RA therapy, a significant portion of patients do not respond adequately to these drugs.

Interleukin-17A (IL-17A) is an inflammatory cytokine produced by TH17 T cells that contributes to the etiology of a number of inflammatory diseases, including RA. IL-17A may exist as either a homodimer or as a heterodimer complexed with its homolog IL-17F to form heterodimeric IL-17A/F. IL-17A is involved in the induction of pro-inflammatory responses and induces or mediates expression of a variety of other cytokines, factors, and mediators including TNF-α, IL-6, IL-8 (CXCL8), IL-1β, granulocyte colony-stimulating factor (G-CSF), prostaglandin E2 (PGE2), IL-10, IL-12, IL-1R antagonist, leukemia inhibitory factor, stromelysin, and nitric oxide. Through its role in T cell mediated autoimmunity, IL-17 is an important local orchestrator of neutrophil accumulation and plays a role in cartilage and bone destruction of a number of inflammatory diseases.

Although a variety of biologics that specifically bind to IL-17 or TNF-α have been produced since the discovery of these cytokines, there remains a need for improved anti-inflammatory drugs that can effectively mediate or neutralize the activity of both IL-17 and TNF-α in the inflammatory response and autoimmune disorders such as RA. A number of bispecific molecules have been developed that can bind both TNF and IL-17. Although shown to be effective in vitro in cell culture models and in vivo in animal models, the effectiveness of such bispecifics for treating humans suffering from inflammatory diseases such as rheumatoid arthritis is unknown.

### BRIEF SUMMARY OF THE INVENTION

The invention provides methods for treating RA in a subject. Such methods comprise administering to a subject one or more binding proteins that bind IL-17 (e.g., IL-17A) and TNF (e.g., TNF-α). In an embodiment, the invention provides methods for treating RA in a human subject using a binding protein that binds to IL-17 and TNF-α. In certain embodiments, the binding protein is a dual variable domain immunoglobulin (DVD-Ig) protein. In certain embodiments, administering the binding protein improves a score of one or more RA metrics. In various embodiments, the subject's RA is resistant to one or more disease-modifying antirheumatic drugs (DMARDs). In certain embodiments, the binding protein is administered concurrently or subsequently with a DMARD. In various embodiments, the DMARD comprises a biologic. In various embodiments, the DMARD comprises a compound such as methotrexate, sulfasalazine, cyclosporine, leflunomide, hydroxychloroquine, or zathioprine. In various embodiments, the binding protein and DMARD are administered concurrently. Alternatively, the binding protein and DMARD are administered at different times (i.e., the DMARD is administered before or after the binding protein is administered).

In various embodiments, the binding protein neutralizes TNF and/or IL-17 in vivo. In various embodiments, the binding protein modulates a negative effect of TNF and/or IL-17 in vivo for a period of time. For example, the period of time is at least four hours, 12 hours, one day, three days, a week, two weeks, three weeks, or a month.

In various embodiments, the binding protein comprises a heavy chain variable region for binding TNF-α comprising the three complementarity determining regions (CDRs) from the amino acid sequence of SEQ ID NO: 12. In various embodiments, the binding protein comprises a heavy chain variable region for binding TNF-α comprising the amino acid sequence of SEQ ID NO: 12. In various embodiments, the binding protein comprises a heavy chain variable region for binding IL-17 comprising the three CDRs from the amino acid sequence of SEQ ID NO: 14. In various embodiments, the binding protein comprises a heavy chain variable region for binding IL-17 comprising the amino acid sequence of SEQ ID NO: 14.

In various embodiments, the binding protein comprises a light chain variable region for binding TNF-α comprising the three CDRs from the amino acid sequence of SEQ ID NO: 17. In various embodiments, the binding protein comprises a light chain variable region for binding TNF-α comprising the amino acid sequence of SEQ ID NO: 17. In various embodiments, the binding protein comprises a light chain variable region for binding IL-17 comprising the three CDRs from the amino acid sequence of SEQ ID NO: 19. In various embodiments, the binding protein comprises a light chain variable region for binding IL-17 comprising the amino acid sequence of SEQ ID NO: 19.

In various embodiments, the binding protein comprises three or six CDR amino acid sequences of the variable heavy chain amino acid sequence of SEQ ID NO: 11. In various embodiments, the binding protein comprises the amino acid sequence of SEQ ID NO: 11 or a portion thereof. In other embodiments, the binding protein comprises three or six CDR amino acid sequences of the variable light chain sequences of SEQ ID NO: 16. In various embodiments, the binding protein comprises the amino acid sequence of SEQ ID NO: 16 or a portion thereof. In an embodiment, the binding protein comprises the amino acid sequence of SEQ ID NO: 11 and the amino acid sequence of SEQ ID NO: 16.

In various embodiments, the binding protein further comprises a constant region. In various embodiments, the constant region comprises the amino acid sequence of SEQ ID NO: 13 or SEQ ID NO: 18. In various embodiments, the constant region comprises at least one mutation compared to a wild-type constant region. In various embodiments, the at least one mutation comprises L240A and/or L241A. In various embodiments, the constant region comprises an Fc. In various embodiments, the Fc region has been inactivated with regards to FcγR binding. In various embodiments, the binding protein comprises at least one mutation in the CH2 or CH3 domain.

In various embodiments, the binding protein is administered subcutaneously. In various embodiments, administering the binding protein is by at least one mode selected from the group consisting of parenteral, subcutaneous, intramuscular, intravenous, intra-articular, intra-abdominal, intra-capsular, intra-cartilaginous, intra-osteal, intrapelvic, intraperitoneal, intrasynovial, intravesical, via bolus, topical, oral, and transdermal.

In various embodiments, the binding protein is administered at least once every day, every other day, every few days, every week, every other week, every three weeks, or every month. For example, the binding protein is administered every two weeks.

In various embodiments, the binding protein is administered at a total dose of between about 0.1-1 milligrams (mg), about 1-5 mg, about 5-10 mg, about 10-15 mg, about 15-20 mg, about 20-25 mg, about 25-50 mg, about 50-75 mg, about 75-100 mg, about 100-125 mg, about 125-150 mg, about 150-175 mg, about 175-200 mg, about 200-225 mg, about 225-250 mg, about 250-275 mg, about 275-300 mg, about 300-325 mg, or about 325-350 mg of the binding protein. In an embodiment, the binding protein is subcutaneously administered weekly at a dose of about 120 milligrams. In various embodiments, the binding protein is subcutaneously administered weekly at a dose of about 15-150 milligrams or about 10-400 mgs. In various embodiments, the binding protein is administered at about 30 mg, about 100 mg, or about 300 mg every other week. In various embodiments, the binding protein is administered at about 30 mg every other week. In various embodiments, the binding protein is administered at about 100 mg every other week. In various embodiments, the binding protein is administered at about 300 mg every other week.

In various embodiments, the binding protein is administered at a dose related to the weight of the patient/subject. For example the dose is calculated in milligrams of binding protein per kilogram of patient weight (mg/kg). In various embodiments, the binding protein is administered at a dose of about: 0.1 mg/kg, 0.3 mg/kg, 1 mg/kg, 1.5 mg/kg; 2 mg/kg; 3 mg/kg, 4 mg/kg; 5 mg/kg; 6 mg/kg; 7 mg/kg; 8 mg/kg; 9 mg/kg; 10 mg/kg; 11 mg/kg; 12 mg/kg; 13 mg/kg; 14 mg/kg; 15 mg/kg; 16 mg/kg; 17 mg/kg; 18 mg/kg; 19 mg/kg; 20 mg/kg; 21 mg/kg; 22 mg/kg; 23 mg/kg; and 24 mg/kg. In various embodiments, the binding protein is formulated for administration to the patient. For example, the binding protein is lyophilized for stability, and then reconstituted with a fluid. For example, the fluid comprises a suspension. In various embodiments, the binding protein is administered using a stock solution at a concentration of about: 50, 75, 100, 120, or 150 milligrams per milliliter. In various embodiments, the binding protein is administered at 0.1 to 24 milligrams per kilograms (mpk). In various embodiments, the binding protein is administered at 0.1 to 24 milligrams per kilograms per day or milligrams per dose (mkd).

In various embodiments, the amount of binding protein administered over the period of time is constant. In various embodiments, the amount of binding protein administered over the period of time is altered. For example, the amount of binding protein is increased from one administration to the following administration. Alternatively, the amount of binding protein is decreased from one administration to the following administration.

In various embodiments, the binding protein that specifically binds both IL-17 and TNF-α is formulated in a pharmaceutical composition comprising a pharmaceutically acceptable carrier. In various embodiments, the method further includes administering to the subject a second agent such as, for example, a therapeutic agent or an imaging agent. For example, the therapeutic agent comprises a DMARD. In certain embodiments, the DMARD is methotrexate. In various embodiments, the second agent is administered concurrently with the binding protein or subsequently. Alternatively in various embodiments, the second agent is administered prior to administering the binding protein.

The subject in various embodiments of the method has been treated with a therapeutic agent (e.g., a DMARD, a steroid, a cyclooxygenase (COX)-2 inhibitor, and acetaminophen) for a period of time prior to administration of the binding protein. In various embodiments, the subject receives a dose of the DMARD of less than about 2 mg, about 5 mg, about 10 mg, about 15 mg or about 20 mg per week. Alternatively, the subject has been administered another therapeutic agent for a period of time of at least two days, a week, two weeks, three weeks, a month, two months, three months, four months, five months, six months or longer.

In various embodiments, the subject is resistant to the therapeutic agent. In various embodiments, the subject is resistant to one or more DMARDs. For example, the patient is resistant to methotrexate.

In various embodiments, the subject has been receiving a methotrexate therapy for a period of time. For example, the period of time is at least about: a day, a few days, a week, two weeks, a month, two months, three months, four months, five months, six months, seven months, eight months, nine months, ten months, eleven months, twelve months, fourteen months, or eighteen months. In various embodiments, the subject is on a stable dose (e.g., about 5-25 mg/week). In various embodiments, the subject is on a stable dose for at least one week, two weeks, three weeks or four weeks prior to the first dose of the binding protein. In various embodiments, the stable dose is about 10 mg/week.

In various embodiments, administration of the binding protein is systemic, is localized to an area of the subject, or diffuses to a treatment area. In various embodiments, the administration is intravenous or by subcutaneous injection.

In various embodiments, the pharmaceutical composition is lyophilized. In various embodiments, the method comprises reconstituting the lyophilized composition prior to administering the binding protein. In various embodiments, the composition comprises at least one of sucrose, histidine, polysorbate, and mineral acid. For example, the mineral acid comprises hydrochloric acid.

In various embodiments of the method, administering the binding protein improves at least one negative condition in the subject associated with RA. In various embodiments, the negative condition is selected from the group consisting of inflammation; stiffness; pain; bone erosion; osteoporosis; joint deformity; a nerve condition (e.g., tingling, numbness, and burning); scarring; a cardiac disorder; a blood vessel disorder; high blood pressure; tiredness; anemia; weight loss; abnormal temperature (e.g., fever); a lung disorder; a kidney disorder; a liver disorder; an ocular disorder; a skin disorder; an intestinal disorder; and an infection.

The method in various embodiments further comprises identifying an improvement in the subject in regards to the severity or duration of a symptom associated with the rheumatoid arthritis. For example, identifying comprises using a score, a test, or a metric for RA or inflammation. In various embodiments of the method, the score, the test, or the metric is selected from the group consisting of: Physician Global Assessment of Disease Activity (Physician Global); Global Arthritis Score; a Patient Global Assessment of Disease Activity (PTGL); Patient Assessment of General Health (GH); Patient Assessment of General Health (GH); patient's assessment of pain; Patient Reported Outcome; global disease activity and physical function; a Health Assessment Questionnaire (HAQ-DI); measurement or presence of an anti-drug antibody (ADA); tender joint count (TJC); swollen joint count (SJC); Work Instability Scale for Rheumatoid Arthritis; Short Form Health Survey (SF-36); American College of Rheumatology (ACR) Criteria (e.g., ACR20, ACR50, and ACR70); ACR Response Rate; proportion of subjects achieving Low Disease Activity (LDA); Disease Activity Score 28 (DAS28; e.g., DAS28 based on C-reactive protein); proportion of subjects achieving ACR70 responder status; Clinical Disease Activity Index (CDAI); simple disease activity index (SDAI); Clinical Remission criteria, and acute phase reactant levels.

In various embodiments, the metric is obtained by questioning the subject or patient regarding their health prior to and after treatment (see any of the tests described in Table 20). In various embodiments, the metric uses at least one test, exam, questionnaire or survey (see for example the regimen of testing described in Table 19).

In various embodiments, the method comprises, prior to administering, collecting a sample from the subject and analyzing and/or detecting at least one biomarker. For example, the biomarker is a protein, peptide, or polynucleotide. In various embodiments, the biomarker is selected from the group consisting of the group selected from: TNF, IL-1Ra, IFNγ, LIF, C-X-C motif chemokine (CXCL) 1, CXCL2, CXCL4, CXCL5, CXCL8, CXCL9, CXCL10, chemokine (C-C motif) ligand 2 (CCL2), CCL23, interleukin-1 beta (IL-1β), IL-6, IL-10, IL-17A, IL-17F, IL-21, IL-22, C-X-C chemokine receptor type 1 (CXCR1), CXCR4, CXCR5, granulocyte-macrophage colony-stimulating factor (GM-CSF), granulocyte-macrophage colony-stimulating factor receptor (GM-CSFR), granulocyte-colony stimulating factor receptor (G-CSFR), granulocyte colony stimulating factor (G-CSF) protein, and a homolog, portion or derivative thereof. In various embodiments, analyzing the biomarker comprises comparing the amount of the biomarker in the subject sample to the amount of biomarker in a second sample from a control subject not having rheumatoid arthritis.

In an embodiment, the invention provides methods for treating a subject having RA, wherein the subject is resistant to treatment with methotrexate, the method comprising the step of administering to the subject a composition comprising a binding protein that specifically binds both IL-17 and TNF-α, wherein the binding protein is a multispecific immunoglobulin, wherein the binding protein is administered at a frequency and dose that improves the score of one or more metrics of RA.

A particular embodiment of the invention provides methods for treating a subject having RA, wherein the subject is resistant to treatment with methotrexate, the method comprising the step of administering to the subject a composition comprising a binding protein that specifically binds both IL-17 and TNF-α, wherein the binding protein is a DVD-Ig protein, and wherein the binding protein comprises at least one polypeptide comprising an amino acid sequence of SEQ ID NO:11 and an amino acid sequence of SEQ ID NO:16, wherein the binding protein is administered weekly and the total amount administered is about 1-400 milligrams of the binding protein. For example, the subject is administered about 20-50 mg, about 50-75 mg, about 75-100 mg, about 100-125 mg, about 125-150 mg, about 150-175 mg, about 175-200 mg, about 200-225 mg, about 225-250 mg, about 250-275 mg, about 275-300 mg, about 300-325 mg, about 325-350 mg, about 350-375 mg, or about 375-400 mg of the binding protein. In various embodiments, the binding protein is administered at a dose of about 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, about 300-325 mg, about 325-350 mg, or about 350-400 mg of the binding protein. In various embodiments, the binding protein is subcutaneously or intravenously administered weekly. In various embodiments, the binding protein is subcutaneously or intravenously administered every other week. In various embodiments, the subject is administered about: 30 mg, 100 mg, 120 mg, 240 mg, or 300 mg every other week.

In an embodiment, the invention provides methods for treating a subject having rheumatoid arthritis, wherein the subject has been treated or is currently being treated with methotrexate, the method comprising the step of administering to the subject that has been treated or is currently being treated with methotrexate a composition comprising a binding protein that specifically binds both IL-17 and TNF-α, wherein the binding protein is a multispecific immunoglobulin, wherein the binding protein is administered at a frequency and dose that improves the score of one or more metrics of rheumatoid arthritis.

An aspect of the invention provides methods of treating a subject having rheumatoid arthritis, wherein the subject has been treated or is currently being treated with methotrexate, the method comprising administering to the subject that has been treated or is currently being treated with methotrexate a binding protein that binds both TNF-α and IL-17, wherein the binding protein is a DVD-Ig binding protein, wherein the binding protein comprises a variable heavy chain comprising the amino acid sequence of SEQ ID NO: 11 and comprises a variable light chain comprising the amino acid sequence of SEQ ID NO: 16, wherein administering the binding protein is performed, for example, using a dose of from 0.005 mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 1.5 mg/kg, from 1.5 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, or from 9 mg/kg to 10 mg/kg of weight of the binding protein to weight of the subject. For example, the binding protein is administered at a dose of about: 0.3 mg/kg, 1.0 mg/kg, or 1.5 mg/kg. In various embodiments of the method, the binding is administered at a dose of about 3.0 mg/kg or about 10 mg/kg. In various embodiments, the binding protein is administered intravenously or subcutaneously. In various embodiments, the binding protein is administered at least once, for example, every day, every other day, every week, every two weeks, every three weeks, every four weeks, and every month. In various embodiments, the binding protein is subcutaneously or intravenously administered every other week.

An aspect of the invention provides methods for treating a subject having RA wherein the subject has or is currently being treated with methotrexate, the method comprising: administering to the subject a binding protein that binds both TNF-α and IL-17, wherein the binding protein is a DVD-Ig binding protein, wherein the binding protein comprises a variable heavy chain comprising the amino acid sequence of SEQ ID NO: 11, and comprises a variable light chain comprising the amino acid sequence of SEQ ID NO: 16, wherein administering the binding protein is performed for example using multiple individual doses to reach the total dose. In various embodiments, the total dose is calculated based on a period of time (e.g., days, week, or weeks). For example, the total dose is between about 1-25 mg, about 25-50 mg, about 50-75 mg, about 75-100 mg, about 100-125 mg, about 125-150 mg, about 150-175 mg, about 175-200 mg, about 200-225 mg, about 225-250 mg, about 250-275 mg, about 275-300 mg, 300-325 mg, about 325-350 mg, 350-375 mg, or 375-400 mg of the binding protein. For example the weekly total dose is about 15 mg, about 50 mg, or about 150 mg. In various embodiments, the binding protein is administered at least once, for example every day, every other day, every week, every two weeks, every three weeks, every four weeks, and every month. In various embodiments, the binding protein is subcutaneously or intravenously administered 30 mg, 100 mg, or 300 mg every other week.

An aspect of the invention provides a method for treating a subject having RA, such that the subject is resistant to treatment with methotrexate, the method comprising the step of administering to the subject a composition comprising a binding protein that specifically binds both IL-17 and TNF-α, and the binding protein is a DVD-Ig protein, and the binding protein comprises at least one polypeptide comprising the amino acid sequence of SEQ ID NO: 11 and the amino acid sequence of SEQ ID NO:16, and the binding protein is administered at from about 10-400 milligrams of the binding protein. For example, the subject is administered about: 30, about 100, or about 300 milligrams of the binding protein. The binding protein in various embodiments of the method is administered every week or every other week. In various embodiments of the method, the binding protein is administered intravenously. The binding protein in various embodiments of the method is administered subcutaneously. In various embodiments, administering the binding protein is by at least one mode selected from the group consisting of: parenteral, subcutaneous, intramuscular, intravenous, intra-articular, intra-abdominal, intra-capsular, intra-cartilaginous, intra-osteal, intrapelvic, intraperitoneal, intrasynovial, intravesical, bolus, topical, oral, and transdermal.

In various embodiments, the method further comprises administering the composition including the binding protein after the methotrexate. Alternatively, the method further comprises administering the composition including the binding protein prior to or currently with the methotrexate.

In various embodiments, the binding protein is administered at a dose of about: 30 mg, 100 mg, or 300 mg. The binding protein in various embodiments of the method is administered at a dosage of about: 0.1 milligram per kilogram of subject weight (mg/kg); 0.3 mg/kg; 1.0 mg/kg; 3 mg/kg; or 10 mg/kg. In various embodiments, the binding protein is administered at 0.1 to 24 milligrams per kilogram. In various embodiments, the binding protein is administered at 0.1 to 24 milligrams per kilograms per day or milligrams per dose. The composition in various embodiments of the method further comprises at least one substance selected from the group consisting of: sucrose, histidine, polysorbate, and mineral acid.

In various embodiments, the binding protein neutralizes TNF-α and/or IL-17. In various embodiments, the binding protein neutralizes TNF-α and/or IL-17 in vivo for a period of time. In various embodiments, the period of time is four hours, 12 hours, one day, two days, three days, four days, ten days, 15 days, 18 days, 21 days, 36 days, 48 days, 60 days, 72 days, or 84 days. In various embodiments, the method further comprises observing modulation of a TNF-mediated or an IL-17-mediated symptom or condition.

In various embodiments, the RA affects one joint, two joints, three joints, four joints, or five joints. In various embodiments, the RA is manifested in the subject in the form of stiffness, pain, swelling, and tenderness of the joints and surrounding ligaments and tendons. In various embodiments, the RA is in a knee, hip, hand, finger, spine/back, toe, and/or foot. In various embodiments, the subject has tendon pain. In various embodiments the subject has at least one joint or nail deformity. In various embodiments, the methods of the invention results in treatment of or amelioration of at least one of the above symptoms.

In various embodiments, the linker comprises SEQ ID NO: 8, SEQ ID NO: 13, or a portion or combination thereof. In various embodiments the linker comprises at least one of SEQ ID NOs: 14-50.

The binding protein in various embodiments comprises a constant region described herein for example in Table 7. For example, the heavy chain constant region comprises the amino acid sequence of SEQ ID NO: 15. For example, the light chain constant region comprises the amino acid sequence of SEQ ID NO: 20.

In various embodiments, the binding protein is about: 30%, 35%, 40%, 45%, 50%, 55%, 60%, 65%, 70%, 75%, 80%, 85%, 90%, 95%, 96%, 98%, or 99% or more identical to the amino acid sequence of SEQ ID NO: 11 and/or SEQ ID NO: 16. In a related embodiment, the binding protein comprises a heavy chain variable region that is about: 30%, 35%, 40%, 45%, 50%, 55%, 60%, 65%, 70%, 75%, 80%, 85%, 90%, 95%, 96%, 98%, or 99% or more identical to the amino acid sequence of SEQ ID NO: 11 and/or a light chain variable region that is about: 30%, 35%, 40%, 45%, 50%, 55%, 60%, 65%, 70%, 75%, 80%, 85%, 90%, 95%, 96%, 98%, or 99% or more identical to the amino acid sequence of SEQ ID NO: 16. In a related embodiment, the binding protein comprises 3 CDRs of a heavy chain variable region that are about: 30%, 35%, 40%, 45%, 50%, 55%, 60%, 65%, 70%, 75%, 80%, 85%, 90%, 95%, 96%, 98%, or 99% or more identical to the three CDRs in the amino acid sequence of SEQ ID NO: 11 and/or a 3 CDRs of a light chain variable region that are about: 30%, 35%, 40%, 45%, 50%, 55%, 60%, 65%, 70%, 75%, 80%, 85%, 90%, 95%, 96%, 98%, or 99% or more identical to the three CDRs in the amino acid sequence of SEQ ID NO: 16.

In various embodiments, the binding protein is formulated in a pharmaceutical composition comprising a pharmaceutically acceptable carrier. In various embodiments, the binding protein is crystallized. In various embodiments, the crystallized binding protein is formulated in a composition comprising an ingredient and/or a polymeric carrier. For example, the polymeric carrier is a polymer selected from the group consisting of poly (acrylic acid), poly (cyanoacrylates), poly (amino acids), poly (anhydrides), poly (depsipeptide), poly (esters), poly (lactic acid), poly (lactic-co-glycolic acid) or PLGA, poly (b-hydroxybutryate), poly (caprolactone), poly (dioxanone); poly (ethylene glycol), poly (hydroxypropyl) methacrylamide, poly [(organo)phosphazene], poly (ortho esters), poly (vinyl alcohol), poly (vinylpyrrolidone), maleic anhydride-alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polysaccharides, blends and copolymers thereof. In various embodiments, the subject is also administered a pain reliever, or a nonsteroidal anti-inflammatory drug (NSAID). In various embodiments, the subject is administered a steroid (e.g., a corticosteroid) or a cyclooxygenase (COX)-2 inhibitor.

In various embodiments, the ingredient is selected from one or more of the group consisting of albumin, sucrose, trehalose, lactitol, gelatin, hydroxypropyl-β-cyclodextrin, methoxypolyethylene glycol and polyethylene glycol.

In various embodiments, the binding protein is formulated in a composition comprising sucrose, histidine, and/or polysorbate 80. In various embodiments, the binding protein is formulated as a powder and water is added to the composition. In various embodiments, the reconstituted solution comprising the binding protein is administered as an injection. In various embodiments, hydrochloric acid added as necessary to adjust pH. In various embodiments, the binding protein is reconstituted with 1.2 milliliters of sterile water for the injection. In various embodiments, the binding protein being reconstituted is at a concentration of about 100 mg/ml.

In various embodiments, the binding protein is administered at a dosage/dose of about: 0.1 milligram per kilogram of subject weight (mg/kg); 0.3 mg/kg; 1.0 mg/kg; 2 mg/kg; 3 mg/kg; 4 mg/kg; 5 mg/kg; 6 mg/kg; 7 mg/kg; 8 mg/kg; 9 mg/kg, or 10 mg/kg. For example, the dose administered is at least about: from 0.005 mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 1.5 mg/kg; from 1.5 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, or from 9 mg/kg to 10 mg/kg of weight of the binding protein to weight of the subject. In various embodiments, the binding protein is administered at a dose of about: 0.1 mg/kg, 0.3 mg/kg, 1.0 mg/kg or 1.5 mg/kg. In various embodiments, the binding protein is administered at a dose of about: 3 mg/kg or 10 mg/kg.

The binding protein may be administered using different regimens and administration schedules. For example, the binding protein may be administered once or a plurality of times (e.g., twice, three times, four times to eight times, eight times to ten times, and ten times to twelve times). For example the administration schedule is determined based on the efficacy and/or tolerability of the binding protein in the subject or subject. In various embodiments, the binding protein is administered at least once, for example every day, every other day, every week, every two weeks, every three weeks, every four weeks, and every month. For example the binding protein is administered every week at a dose of about: 0.3 mg/kg, 1.0 mg/kg, 1.5 mg/kg, 3 mg/kg, or 10 mg/kg. In various embodiments, the binding protein is administered at a weekly total dose of about 10-400 mg. In an embodiment, the binding protein is subcutaneously administered weekly or every other week at a dose of about 60-300 mg. For example, the binding protein is administered 100-300 mg (e.g., 120 mg and 200 mg) every other per week.

In various embodiments, the subject has been treated with a DMARD for a period of time prior to administration of the binding protein such that the subject has become resistant to the treatment/therapy. For example, the resistance is least about: 1%-10%, 10%-20%, 20%-30%, 30%-40%, 40%-50%, 50%-60%, 60%-70%, 70%-80%, 90-95%, or 95%-99%, resistance to one or more DMARD activities. In various embodiments, the binding protein modulates and reduces the level of resistance by about: 1%-10%, 10%-20%, 20%-30%, 30%-40%, 40%-50%, 50%-60%, 60%-70%, 70%-80%, 90-95%, or 95%-99%.

In various embodiments, the method further includes administering the binding protein after administering the DMARD, e.g., methotrexate. Alternatively, the method involves administering the binding protein prior to or concurrently with the DMARD. In a related embodiment of the method, administering the binding protein improves at least one negative condition or symptom in the subject associated with RA. In various embodiments, the at least one RA-associated symptom or condition is selected from the group consisting of: autoimmune response (e.g., antibodies and adverse effects); inflammation; stiffness; pain; bone erosion/osteoporosis; joint deformity; joint destruction, a nerve condition (e.g., tingling, numbness, and burning); scarring; a cardiac disorder/condition; a blood vessel disorder/condition; high blood pressure; tiredness; anemia; weight loss; an abnormal temperature (e.g., elevated); a lung condition/disease; a kidney condition/disorder; a liver condition/disorder; an ocular disorder/condition; a skin disorder/condition; an intestinal disorder/condition; and an infection.

In various embodiments, administration of the binding protein to the subject improves a score of one or more RA metrics or criteria in the subject. For example, RA metric and criteria are described in the Examples herein. In various embodiments, the RA metric is selected from the group consisting of one or more of an: Physician Global Assessment of Disease Activity (Physician Global); Global Arthritis Score; a Patient Global Assessment of Disease Activity (PTGL); Patient Assessment of General Health (GH); Patient Assessment of General Health (GH); patient's assessment of pain; Patient Reported Outcome; global disease activity and physical function; a Health Assessment Questionnaire (HAQ-DI); measurement or presence of an anti-drug antibody (ADA); tender joint count (TJC); swollen joint count (SJC); Work Instability Scale for Rheumatoid Arthritis; Short Form Health Survey (SF-36); American College of Rheumatology (ACR) Criteria (e.g., ACR20, ACR50, and ACR70); ACR Response Rate; proportion of subjects achieving Low Disease Activity (LDA); Disease Activity Score 28 (DAS28; e.g., DAS28 based on C-reactive protein); proportion of subjects achieving ACR70 responder status; Clinical Disease Activity Index (CDAI); simple disease activity index (SDAI); Clinical Remission criteria, and acute phase reactant levels. For example, the binding protein reduces and/or modulates the RA metric or criteria by at least about 1%, 3%, 5%, 7%, 10%, 15%, 20%, 25%, 30%, 35%, 40%, 45%, 50%, 55%, 60%, 65%, 70%, 75%, 80%, 85%, 90%, 95%, 99% or more.

In various embodiments, the biomarker is selected from the group consisting of the group selected from: TNF, IL-1Ra, IFNγ, LIF, CXCL 1, CXCL2, CXCL4, CXCL5, CXCL8, CXCL9, CXCL10, CCL2, CCL23, interleukin-1 beta (IL-1(3), IL-6, IL-10, IL-17A, IL-17F, IL-21, IL-22, CXCR1, CXCR4, CXCR5, GM-CSF, GM-CSFR, G-CSFR, G-CSF protein, and a homolog, portion or derivative thereof.

In various embodiments, the method further comprises observing or detecting a modulation (i.e., a reduction or an increase) in presence or activity of the biomarker. In various embodiments, the biomarker is selected from the group consisting of: IL-1Ra, GM-CSF, TNF, IL-10, IFNγ, IL-21, LIF, CXC4, CXCR5, a high-sensitivity C-reactive protein (hsCRP); a matrix metallopeptidase (MMP; for example MMP-9); a vascular endothelial growth factor (VEGF), a MMP degradation product for example MMP degradation product of type I, II, or III collagen (C1M, C2M, C3M); a C-reactive protein (CRPM), a prostaglandin, nitric oxide, a disintegrin and metalloproteinase with thrombospondin motifs (ADAMTS), an adipokine, an endothelial growth factor (EGF), a bone morphogenetic protein (BMP), a nerve growth factor (NGF), a substance P, an inducible Nitric Oxide Synthase (iNOS), CTX-I, CTX-II, TIINE, creatinine, and a vimentin (for example a citrullinated and MMP-degraded vimentin; VICM). In various embodiments of the method, the binding protein reduces the arthritis and/or modulates (e.g., reduces and increases) expression and/or activity of the biomarker by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

In various embodiments of the method, the binding protein reduces the arthritis and/or modulates (e.g., reduces and increases) expression and/or activity of the biomarker by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

In various embodiments, the method further comprises administering the composition including the binding protein after having administered the methotrexate. In various embodiments, the method further comprises administering another agent to the subject. For example, the additional agent is selected from the group consisting of: therapeutic agent, imaging agent, cytotoxic agent, angiogenesis inhibitors; kinase inhibitors; co-stimulation molecule blockers; adhesion molecule blockers; anti-cytokine antibody or functional fragment thereof; methotrexate; cyclosporin; rapamycin; FK506; detectable label or reporter; a TNF antagonist; an antirheumatic; a muscle relaxant, a narcotic, NSAID, an analgesic, an anesthetic, a sedative, a local anesthetic, a neuromuscular blocker, an antimicrobial, an antipsoriatic, a corticosteroid, an anabolic steroid, an erythropoietin, an immunization, an immunoglobulin, an immunosuppressive, a growth hormone, a hormone replacement drug, a radiopharmaceutical, an antidepressant, an antipsychotic, a stimulant, an asthma medication, a beta agonist, an inhaled steroid, an epinephrine or analog, a cytokine, and a cytokine antagonist. Alternatively, the binding protein is administered concurrently or prior to administering the additional agent.

Methods are provided for treating rheumatoid arthritis and/or other inflammatory diseases using any of the binding proteins described herein that are capable of binding TNF and IL-17 with high affinity. An aspect of the invention provides a method for reducing a symptom of rheumatoid arthritis and/or an inflammatory disorder in a subject in need thereof comprising administering to the subject a binding protein that specifically binds both human IL-17 and TNF-α. For example, the binding protein comprises at least one amino acid sequence of SEQ ID NOs: 1-20. In various embodiments of the method, the binding protein comprises at least one amino acid sequence of SEQ ID NOs: 11-20 or a portion or combination thereof.

In various embodiments, the binding protein comprises three complementarity determining regions (CDRs) in a heavy chain variable domain found in amino acid sequence of SEQ ID NO: 12 or SEQ ID NO: 14. In various embodiments of the method, the binding protein comprises a heavy polypeptide chain of the formula VD1-(X1)n-VD2-C-(X2)n, wherein;

VD1 is a first heavy chain variable domain;

VD2 is a second heavy chain variable domain;

C is a heavy chain constant domain;

X1 is a linker with the proviso that it is not CH1;

X2 is an Fc region; and

n is 0 or 1;

wherein VD1 comprises the amino acid sequence of SEQ ID NO: 12 or SEQ ID NO:14.

In various embodiments of the method, n is 0 or 1 and X1 is a polypeptide comprising the amino acid sequence SEQ ID NO: 13. The heavy polypeptide chain in various embodiments of the method comprises the amino acid sequence SEQ ID NO: 11.

In various embodiments of the method, the binding protein comprises three CDRs in a light chain variable domain found in amino acid sequence of SEQ ID NO: 17 and SEQ ID NO: 19. In various embodiments, the binding protein comprises a light polypeptide chain, of the formula VD1-(X1)n-VD2-C-(X2)n, wherein;

VD1 is a first light chain variable domain;

VD2 is a second light chain variable domain;

C is a light chain constant domain;

X1 is a linker with the proviso that it is not CL;

X2 does not comprise an Fc region; and

n is 0 or 1;

wherein VD1 comprises the amino acid sequence of SEQ ID NO: 17 or SEQ ID NO:19.

In various embodiments of the method, n is 0 or 1 and the X1 of the first light chain variable domain comprises SEQ ID NO: 18. In various embodiments, the light polypeptide chain comprises the amino acid sequence SEQ ID NO: 16.

In various embodiments of the method, the binding protein comprises two heavy polypeptide chains and two light polypeptide chains. In various embodiments, the binding protein further comprises an Fc region or a mutated Fc region compared to a wild-type Fc region. For example, the Fc region comprises an amino acid sequence selected from SEQ ID NO: 15 and SEQ ID NO: 20.

In various embodiments of the method, the binding protein is a DVD-Ig binding protein. For example comprising a variable heavy chain domain amino acid sequence of SEQ ID NO: 11 and a variable light chain domain amino acid sequence of SEQ ID NO: 16. In various embodiments, the binding protein is a DVD-Ig binding protein, for example comprising a variable heavy chain domain amino acid sequence of SEQ ID NO: 5 and a variable light chain domain amino acid sequence of SEQ ID NO: 8. In various embodiments of the method, the arthritis comprises RA. In various embodiments, the subject is a mammal, for example a rodent. In various embodiments of the method, the subject is a human.

The binding protein is a DVD-Ig binding protein, and for example the binding protein is administered subcutaneously or parenterally. An aspect of the invention provides a method for treating rheumatoid arthritis in a human subject comprising the step of administering to the human subject a binding protein that specifically binds both TNF-α and IL-17, wherein the binding protein is a DVD-Ig binding protein including a variable heavy chain comprising an amino acid sequence of SEQ ID NO: 11, and including a variable light chain comprising an amino acid sequence of SEQ ID NO: 16, in a dose to achieve: an area under the curve (AUC) of between about 1 and about 2000 μg·day/mL; an AUCinf/dose of about 100 to about 1000 μg·day/mL per mg/kg; a serum or plasma half-life (T1/2) of an area under the curve from 0 to 14 days (AUC₀₋₄₄) at least about between about 120,000 and 160,000 mg/kg; a time point to maximum observed serum concentration (Tmax) of between about 2 to 960 hours; a Tmax of between 2 to 40 days; a maximum observed serum concentration (Cmax) of between about 0.5 to 400 μg/mL; a maximum observed serum concentration (Cmax) per dose of between about 2 to 100 μg/mL per mg/kg; a clearance of about 0.05 to 10 liters per day (L/day); and a serum half-life (t_{1/2}) of about 1 to 15 days; a maximum observed serum concentration (Cmax) of between about 12,000 and 16,000 μg/mL; an improvement of a negative condition or symptom associated with rheumatoid arthritis; and/or an improvement in a score or criteria of at least one rheumatoid arthritis metric.

In various embodiments, the AUC is between about 75 and about 1600 μg·day/mL. (for example about 105 to 1500 μg·day/mL, or about 105 to 500 μg·day/mL. In various embodiments, the AUCinf/dose is about 250 to 750 μg·day/mL per mg/kg. In various embodiments, the serum or plasma half-life (T1/2) of an area under the curve from 0 to 14 days (AUC₀₋₁₄) at least about between about 140,000 and 150,000 mg/kg. In various embodiments, the Tmax is about 4-300 hours. In various embodiments, the Tmax is about 6-12 days. In various embodiments, the Cmax is about 8 to 100 μg/mL. In various embodiments, the Cmax per dose is about 10 to 40 μg/mL per mg/kg. In various embodiments, the clearance rate is about 0.2 to 1 L/day. In various embodiments, the serum half-life is about 3 to 20 days. In various embodiments, the NOAEL dose was 200 mg/kg IV and resulted in a Cmax and stimulated AUC of 0-14 day of 14,600 μg/mL and 147,500 μg·day/mL, respectively. The estimated AUC at the NOAEL provide 1222- and 122-fold safety margin relative to the steady-state AUC at the starting dose of 30 mg/kg EOW and highest dose of 300 mg/kg EOW, respectively.

In various embodiments of the method, the negative condition or symptom is selected from the group consisting of: autoimmune response; inflammation; stiffness; pain; bone erosion; osteoporosis; joint deformity; joint destruction, a nerve condition; scarring; a cardiac disorder; a blood vessel disorder; high blood pressure; tiredness; anemia; weight loss; an abnormal temperature; a lung disorder; a kidney disorder; a liver disorder; an ocular disorder; a skin disorder; an intestinal disorder; and an infection. In an embodiment, the binding protein reduces the negative symptom by about: 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

In various embodiments, the rheumatoid arthritis metric is selected from the group consisting of: Physician Global Assessment of Disease Activity (Physician Global); Global Arthritis Score; a Patient Global Assessment of Disease Activity (PTGL); Patient Assessment of General Health (GH); Patient Assessment of General Health (GH); patient's assessment of pain; Patient Reported Outcome; global disease activity and physical function; a Health Assessment Questionnaire (HAQ-DI); measurement or presence of an anti-drug antibody (ADA); tender joint count (TJC); swollen joint count (SJC); Work Instability Scale for Rheumatoid Arthritis; Short Form Health Survey (SF-36); American College of Rheumatology (ACR) Criteria (e.g., ACR20, ACR50, and ACR70); ACR Response Rate; proportion of subjects achieving Low Disease Activity (LDA); Disease Activity Score 28 (DAS28; e.g., DAS28 based on C-reactive protein); proportion of subjects achieving ACR70 responder status; Clinical Disease Activity Index (CDAI); simple disease activity index (SDAI); Clinical Remission criteria, and acute phase reactant levels. In various embodiments, the metric is obtained using an assay or test (see any of the tests described in Table 20). In various embodiments, the metric uses at least one test, exam, questionnaire or survey (see for example the regimen or variations of testing described in Table 19).

In an embodiment, the binding protein reduces the metric by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

The subject in various embodiments of the method is resistant to treatment with at least one DMARD. For example, the DMARD is selected from the group consisting of methotrexate, sulfasalazine, cyclosporine, leflunomide, hydroxychloroquine, and zathioprine.

In various embodiments of the method, administering the binding protein is by at least one mode selected from the group consisting of: parenteral, subcutaneous, intramuscular, intravenous, intra-articular, intra-abdominal, intra-capsular, intra-cartilaginous, intra-osteal, intrapelvic, intraperitoneal, intrasynovial, intravesical, bolus, topical, oral, and transdermal. In various embodiments, the binding protein is administered every day, every two days, twice per week, once per week, every two weeks, every three weeks, every month, every two months, or every few months. In various embodiments, the binding protein is administered in a single dose. In various embodiments, the binding protein is administered in multiple doses.

In various embodiments, the method further comprises administering another therapeutic agent. In various embodiments of the method, the therapeutic agent comprises a DMARD.

In various embodiments, the binding protein is administered at a dosage from the group consisting of: 0.1 milligram per kilogram of subject weight (mg/kg); 0.3 mg/kg; 1.0 mg/kg; 1.5 mg/kg; 2 mg/kg; 3 mg/kg; 4 mg/kg; 5 mg/kg; 6 mg/kg; 7 mg/kg; 8 mg/kg; 9 mg/kg; 10 mg/kg; 11 mg/kg; 12 mg/kg; 13 mg/kg; 14 mg/kg; 15 mg/kg; 16 mg/kg; 17 mg/kg; 18 mg/kg; 19 mg/kg; 20 mg/kg; 21 mg/kg; 22 mg/kg; 23 mg/kg; and 24 mg/kg. For example, the binding protein is administered at a dose selected from the group consisting of: from about 0.5 mg/kg, 1 mg/kg, 1.5 mg/kg, 3 mg/kg, and 10 mg/kg.

In various embodiments, the binding protein is administered at a dose from the group consisting of about: 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, about 325-350 mg, 350-375 mg, or 375-400 mg of the binding protein. For example, the dose comprises a dose described herein. In various embodiments, the dose is at least about from 60 mg, 120 mg, 200 mg, or 240 mg. In various embodiments, the dose is administered weekly or every other week. In various embodiments, the binding protein is administered at 0.1 to 24 milligrams per kilograms. In various embodiments, the binding protein is administered at 0.1 to 24 milligrams per kilograms per day. For example, the dose is about 0.3 mg/kg, 1 mg/kg, 1.5 mg/kg, 3 mg/kg, or 10 mg/kg.

### BRIEF DESCRIPTION OF THE DRAWINGS

FIG. 1A is a protocol for a mouse collagen induced arthritis (CIA) model involving injecting collagen II and complete Freund's adjuvant (CFA) into subjects at day zero. In addition subjects were either administered a prophylactic dosing of anti-TNF antibody, anti-IL-17 antibody or anti-TNF/anti-IL-17 DVD-Ig protein (at day 20 after collagen II/CFA injection) one day prior to injection of one milligram of zymosan (at day 21 after collagen II/CFA injection). For another group, a therapeutic dose of anti-TNF antibody, anti-IL-17 antibody or anti-TNF/anti-IL-17 DVD-Ig protein was administered to subjects (at days 24-28 after collagen II/CFA injection) three to seven days after an injection of zymosan (at day 21 after collagen II/CFA injection). Paw swelling (millimeter cubed divided by mean arthritis score; mm³/MAS) was analyzed using calipers over a period of days.

FIG. 1B is a graph showing mean arthritic score (ordinate) as a function of time (abscissa) of subjects in a CIA model administered a prophylactic dose of antibodies. The murine subjects were administered either 8C11 anti-TNF antibody (Ab); MAB421 anti-IL-17 Ab; or a mixture of both 8C11 anti-TNF Ab and MAB421 anti-IL-17 Ab. Control subjects were administered vehicle only.

FIG. 1C is a graph showing mean arthritic score (ordinate; millimeter cubed; mm³) as a function of time (abscissa) of subjects in a CIA model administered a therapeutic dose of antibodies. The murine subjects were administered either 8C11 anti-TNF Ab; MAB421 anti-IL-17 Ab; or a mixture of both 8C11 anti-TNF Ab and MAB421 anti-IL-17 Ab. Control subjects were administered vehicle only. The MAS was calculated over 21 days of disease in the CIA model. Treatment groups: vehicle, 12 mg/kg anti-TNF Ab, 12 mg/kg of anti-IL-17 Ab, or 12 mg/kg each of anti-TNF Ab+anti-IL-17 Ab.

FIG. 1D includes both a representative micro-CT of tarsal bone from naïve or arthritic animals treated with vehicle or antibodies as indicated (FIG. 1D top), and a graph showing micro CT analyzed bone volume (mm³; ordinate) of tarsal bone of subjects in a CIA model administered a dose of antibodies (FIG. 1D bottom). The subjects were administered either 8C11 anti-TNF Ab; MAB421 anti-Il-17 Ab; or a mixture of both 8C11 anti-TNF Ab and MAB421 anti-IL-17 Ab. Control subjects were administered vehicle only. Naïve subjects were not administered a dose.

FIG. 1E is a graph showing histological scores (ordinate) of rear paws of subjects in a CIA model administered a dose of antibodies. The subjects were administered either 8C11 anti-TNF Ab; MAB421 anti-IL-17 Ab; or a mixture of both 8C11 anti-TNF Ab and MAB421 anti-IL-17 Ab. Control subjects were administered vehicle only.

FIG. 1F is a bar graph showing area under the curve (AUC) measured using mean arthritic score (MAS) of rear paws of subjects in a CIA model administered a dose of antibodies. The subjects were administered either 8C11 anti-TNF Ab; MAB421 anti-IL-17 Ab; or a mixture of both 8C11 anti-TNF Ab and MAB421 anti-IL-17 Ab. Control subjects were administered vehicle only. The graph shows quantification of percent (%) inhibition by comparison of the AUC.

FIG. 2 is a graph showing percent inhibition of paw swelling (ordinate) for subjects in a CIA model that were administered different doses (mg/kg) of 8C11 anti-TNF Ab or a mixture of both 8C11 anti-TNF Ab and MAB421 anti-IL-17 Ab. The ED₅₀ is the mean effective dose; ED₅₀ for the anti-TNF Ab is 0.23 mg/kg and the ED₅₀ for the combination of anti-TNF Ab and anti-IL-17 Ab is 0.14 mg/kg. Dose-dependent inhibition of the AUC of paw swelling in the mouse CIA model through day 7 was observed. Black circles represent data for mice treated with anti-TNF Ab alone. Triangles represent data for mice treated with anti-TNF Ab dose in combination with 6 mg/kg of anti-Il-17 Ab.

FIG. 3A is a graph showing change in paw thickness (ordinate; change in millimeters) as a function of time in subjects administered 8C11/10F7M11 anti-TNF/anti-IL-17 DVD-Ig protein (abscissa). Control subjects were administered vehicle only.

FIG. 3B is a graph showing AUC of change in paw thickness (ordinate; millimeters) in subjects administered 8C11/10F7M11 anti-TNF/anti-IL-17 DVD-Ig protein (abscissa). Control subjects were administered vehicle only.

FIG. 3C is a graph showing histology score (ordinate) for inflammation, cartilage, and bone in subjects administered 8C11/10F7M11 anti-TNF/anti-IL-17 DVD-Ig protein. Control subjects were administered vehicle only.

FIG. 3D is a graph showing bone volume (ordinate; millimeters cubed, mm³) in subjects administered 8C11/10F7M11 anti-TNF/anti-IL-17 DVD-Ig protein. Control subjects were administered vehicle only. Naïve subjects were not administered a DVD-Ig protein or vehicle.

FIG. 4A is a graph showing serum concentration (μg/ml; ordinate) of ABBV-257 as a function of time (abscissa; hours) for mice intravenously administered the TNF/IL-17 DVD-Ig binding protein (5 mg/kg). Serum exposure was maintained in 4/6 mice Animals with apparent ADA (*) were excluded from pharmacokinetic calculations.

FIG. 4B is a graph showing serum concentration (μg/ml; ordinate) of ABBV-257 as a function of time (abscissa; hours) for Sprague Dawley rats (numbers 1-5) intravenously administered the TNF/IL-17 DVD-Ig binding protein (5 mg/kg). Serum exposure was maintained in 5/5 rats.

FIG. 5 is a graph showing serum concentration values (μg/ml; ordinate) as function of time (abscissa; hours) for female cynomolgus monkeys following weekly 100 mg/kg intravenous infusion doses of ABBV-257 (4 doses total) followed by a five week washout period (n=4 per group). The terminal half-life observed after the fourth dose was 13.0 days.

FIG. 6A is a graph showing serum concentrations of ABBV-257 (μg/mL; ordinate) as a function of time (abscissa; hours) for cynomolgus monkeys administered weekly intravenous doses (60 or 200 mg/kg) of the binding protein or administered weekly subcutaneous doses (200 mg/kg) of the binding protein. N=6 per dose group. The intravenous administration involved a continuous infusion over two hours. The mean (±SD) concentrations are shown. N=4 to 8. Samples were obtained after dosing on D1 (Day 1), D22 (Day 22), and D50 (Day 50).

FIG. 6B is a graph showing trough concentrations of ABBV-257 (μg/mL; ordinate) as a function of time (abscissa; days) in cynomolgus monkeys administered weekly intravenous doses (60 mg/kg, square; or 200 mg/kg, circle) of the binding protein or administered weekly subcutaneous doses (200 mg/kg, triangle) of the binding protein. N=6 per dose group. The mean (±SD) concentrations are shown. N=4 to 8. Samples were obtained after dosing on D1 (Day 1), D22 (Day 22), and D50 (Day 50).

FIG. 7A is a graph showing serum concentrations of ABBV-257 (μg/mL; ordinate) as a function of time (abscissa; days) for human patients intravenously administered a single dose (0.3 mg/kg, 1.0 mg/kg, or 3.0 mg/kg) of ABBV-257 binding protein.

FIG. 7B is a graph showing serum concentrations of ABBV-257 (μg/mL; ordinate) as a function of time (abscissa; days) for human patients subcutaneously administered a single dose (0.3 mg/kg or 3.0 mg/kg) of ABBV-257 binding protein.

### DETAILED DESCRIPTION OF THE INVENTION

Rheumatoid arthritis (RA) is an autoimmune disease that produces a number of symptoms in subjects, including inflammation, redness, swelling, and pain. During the inflammation process, the normally thin synovium thickens and makes the joint swollen, puffy, and sometimes warm to the touch. As rheumatoid arthritis progresses, the inflamed synovium invades and destroys the cartilage and bone within the joint. The surrounding muscles, ligaments, and tendons that support and stabilize the joint become weak and unable to work normally. These effects lead to the pain and joint damage often seen in rheumatoid arthritis. DMARDs are often used to treat these effects; however over time some patients fail to effectively respond to the DMARDs, e.g., the patient becomes resistant.

Pro-inflammatory cytokines tumor necrosis factor (TNF, also known as TNF-α) and Interleukin 17 (IL-17) have important roles in the pathogenesis of RA. Both cytokines are expressed at increased levels in synovial tissue and are key factors in the joint inflammation and damage to bone and cartilage that are hallmarks of the disease. This invention pertains to methods of using binding proteins, or antigen-binding portions thereof, that bind to IL-17 and TNF-α, to treat RA, RA-associated symptoms and/or DMARD-resistant RA.

Methods of improving symptoms associated with rheumatoid arthritis are provided herein. These methods encompass using binding proteins to specifically bind TNF and IL-17. In certain embodiments, these binding proteins neutralize at least one activity associated with TNF or IL-17. In some embodiments, two binding proteins are used: a first binding protein that specifically binds TNF and a second binding protein that specifically binds IL-17. In other embodiments one binding protein is used that specifically binds both TNF and IL-17.

In certain embodiments, the binding protein is a dual variable domain (DVD) immunoglobulin (DVD-Ig) binding protein. ABBV-257 is a DVD-Ig binding protein that specifically binds and neutralizes TNF-α and IL-17 and prevents them from binding to their respective receptors on cells. These proteins are described in greater detail herein. In other embodiments, other binding proteins can be used including antibodies and fragments thereof.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including”, as well as other forms, such as “includes” and “included”, is not limiting. Terms such as “element” or “component” encompass both elements and components comprising one unit and elements and components that comprise more than one subunit unless specifically stated otherwise.

Generally, nomenclatures used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients. For example, formulations and methods of producing and making compositions using a binding protein (e.g., a DVD-Ig protein) are described in U.S. publication number 20140161817; U.S. Pat. No. 8,835,610; and U.S. Pat. No. 8,779,101, each of which is incorporated by reference herein in its entirety. Select terms are defined below:

The term “biological activity” means all inherent biological properties of a molecule.

A “disease-modifying anti-rheumatic drug” (DMARD) means a drug or agent that modulates, reduces or treats the symptoms and/or progression associated with an immune system disease, including autoimmune diseases (e.g., rheumatic diseases), graft-related disorders and immunoproliferative diseases. The DMARD may be a synthetic DMARD (e.g., a conventional synthetic disease modifying antirheumatic drug) or a biologic DMARD. For example, the DMARD used may be a methotrexate, a sulfasalazine (Azulfidine), a cyclosporine (Neoral®, Sandimmune®), a leflunomide (Arava®), a hydroxychloroquine (Plaquenil®), a Azathioprine (Imuran®), or a combination thereof. In various embodiments, a DMARD is used to treat or control progression, joint deterioration, and/or disability associated with RA.

The term “polypeptide” means any polymeric chain of amino acids and encompasses native or artificial proteins, polypeptide analogs or variants of a protein sequence, or fragments thereof, unless otherwise contradicted by context. A polypeptide may be monomeric or polymeric. For an antigenic polypeptide, a fragment of a polypeptide optionally contains at least one contiguous or nonlinear epitope of a polypeptide. The precise boundaries of the at least one epitope fragment can be confirmed using ordinary skill in the art.

The term “variant” means a polypeptide that differs from a given polypeptide in amino acid sequence by the addition, deletion, or conservative substitution of amino acids, but that retains the biological activity of the given polypeptide (e.g., a variant TNF-α can compete with anti-TNFα antibody for binding to TNF). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index, hydrophilicity of amino acids, as is understood in the art. The term “variant” encompasses a polypeptide or fragment thereof that has been differentially processed, such as by proteolysis, phosphorylation, or other post-translational modification, yet retains its biological activity or antigen reactivity, e.g., the ability to bind to TNF-α and IL-17.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a protein or polypeptide that is chemically synthesized or synthesized in a cellular system different from the cell from which it naturally originates is isolated from its naturally associated components. A protein or polypeptide may also be rendered substantially free of naturally associated components by isolation using protein purification techniques well known in the art.

The term “human IL-17” (“hIL-17”) includes a homodimeric protein comprising two 15 kD IL-17A proteins (hIL-17A/A) and a heterodimeric protein comprising a 15 kD IL-17A protein and a 15 kD IL-17F protein (“hIL-17A/F”). The amino acid sequences of hIL-17A and hIL-17F are shown in Table 1. The term “hIL-17” includes recombinant hIL-17 (rhIL-17), which can be prepared by standard recombinant expression methods.

The term “human TNF-α” (“hTNF-α”, or “hTNF”) means a 17 kD secreted form and a 26 kD membrane associated form of a human cytokine, the biologically active form of which is composed of a trimer of noncovalently bound 17 kD molecules. The structure of hTNF-α is described further in, for example, Pennica et al. (1984) Nature 312:724-729; Davis et al. (1987) Biochem. 26:1322-1326; and Jones et al. (1989) Nature 338:225-228. The term hTNF-α includes recombinant human TNF-α (“rhTNF-α”). The amino acid sequence of hTNF-α is shown in Table 1.

TABLE 1
Sequence of Human IL-17A and Human TNF-α

| | | Sequence |
|---|---|---|
| | Sequence | 1234567890123456789012345678901234 |
| Protein | Identifier | 567890 |
| Human IL- | SEQ ID | GITIPRNPGCPNSEDKNFPRTVMVNLNIHNRNTN |
| 17A | NO.: 64 | TNPKRSSDYYNRSTSPWNLHRNEDPERYPSVIWEAKCRHL |
| | | GCINADGNVDYHMNSVPIQQEILVLRREPPHCPNSFRLEK |
| | | ILVSVGCTCVTPIVHHVA |
| Human IL- | SEQ ID | RKIPKVGHTFFQKPESCPPVPGGSMKLDIGIINE |
| 17F | NO.: 65 | NQRVSMSRNIESRSTSPWNYTVTWDPNRYPSEVVQAQCRN |
| | | LGCINAQGKEDISMNSVPIQQETLVVRRKHQGCSVSFQLE |
| | | KVLVTVGCTCVTPVIHHVQ |
| Human | SEQ ID | MSTESMIRDVELAEEALPKKTGGPQGSRRCLFLS |
| TNF-α | NO.: 66 | LFSFLIVAGATTLFCLLHFGVIGPQREEFPRDLSLISPLA |
| | | QAVRSSSRTPSDKPVAHVVANPQAEGQLQWLNRRANALLA |
| | | NGVELRDNQLVVPSEGLYLIYSQVLFKGQGCPSTHVLLTH |
| | | TISRIAVSYQTKVNLLSAIKSPCQRETPEGAEAKPWYEPI |
| | | YLGGVFQLEKGDRLSAEINRPDYLDFAESGQVYFGIIAL |

The term “IL-17/TNF-α binding protein” means a bispecific binding protein (e.g., DVD-Ig protein) that binds IL-17 and TNF-α.

The terms “specific binding” or “specifically binding,” in reference to the interaction of an antibody, a protein, or a peptide with a second chemical species, mean that the interaction is dependent upon the presence of a particular structure (e.g., an antigenic determinant or epitope) on the chemical species. If an antibody is specific for epitope “A”, in the presence of a molecule containing epitope A (or free, unlabeled epitope A) in which “A” is labeled, the antibody reduces the amount of labeled A bound to the antibody.

The term “antibody” means any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art, and nonlimiting embodiments thereof are discussed herein.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG 1, IgG2, IgG 3, IgG4, IgA1 and IgA2), or subclass.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, a CH2 domain and a CH3 domain, and optionally comprises a CH4 domain. Replacements of amino acid residues in the Fc portion to alter antibody effector function are known in the art (U.S. Pat. Nos. 5,648,260 and 5,624,821). The Fc portion of an antibody mediates several important effector functions, e.g., cytokine induction, ADCC, phagocytosis, complement dependent cytotoxicity (CDC), and half-life/clearance rate of antibody and antigen-antibody complexes. In some cases these effector functions are desirable for a therapeutic antibody but in other cases might be unnecessary or even deleterious, depending on the therapeutic objectives. Certain human IgG isotypes, particularly IgG1 and IgG3, mediate ADCC and CDC via binding to FcγRs and complement Clq, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered. The dimerization of two identical heavy chains of an immunoglobulin is mediated by the dimerization of CH3 domains and is stabilized by the disulfide bonds within the hinge region (Huber et al. 1976 Nature 264: 415-20; Thies et al., 1999 J. Mol. Biol. 293: 67-79). Mutation of cysteine residues within the hinge regions to prevent heavy chain-heavy chain disulfide bonds destabilizes dimerization of CH3 domains. Residues responsible for CH3 dimerization have been identified (Dall'Acqua 1998Biochem. 37: 9266-73). Therefore, it is possible to generate a monovalent half-Ig. Interestingly, these monovalent half Ig molecules have been found in nature for both IgG and IgA subclasses (Seligman 1978 Ann. Immunol. 129: 855-70; Biewenga et al., 1983 Clin. Exp. Immunol. 51: 395-400). The stoichiometry of FcRn: Ig Fc region has been determined to be 2:1 (West et al. 2000 Biochem. 39: 9698-708), and half Fc is sufficient for mediating FcRn binding (Kim et al., 1994 Eur. J. Immunol. 24: 542-548). Mutations to disrupt the dimerization of CH3 domain may not have greater adverse effect on its FcRn binding as the residues important for CH3 dimerization are located on the inner interface of CH3 β sheet structure, whereas the region responsible for FcRn binding is located on the outside interface of CH2-CH3 domains. However the half Ig molecule may have certain advantages in tissue penetration due to its smaller size than that of a regular antibody. In one embodiment at least one amino acid residue is replaced in the constant region of the binding proteins provided herein, for example the Fc region, such that the dimerization of the heavy chains is disrupted, resulting in half DVD-binding protein molecules. The anti-inflammatory activity of IgG is dependent on sialylation of the N-linked glycan of the IgG Fc fragment. The precise glycan requirements for anti-inflammatory activity has been determined, such that an appropriate IgG1 Fc fragment can be created, thereby generating a fully recombinant, sialylated IgG1 Fc with greatly enhanced potency (Anthony et al., 2008 Science 320: 373-376). Exemplary constant regions are shown below.

```
Wild type hIgG1 constant region (SEQ ID NO: 60):
          ASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEV
TCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFL
YSKLTVDKSRWQQGNVFSCSVMHEALHNHYTQKSLSLSPGK.
Mutant hIgG1 constant region (SEQ ID NO: 61)
          ASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTK
VDEKVEPESCDETHTCPPCPAPEAAGGPSVFLFPPKPKDTLMISRTPEV
TCVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSREE
MTKNQVSLTCLVKGFYPSDIAVEWESNGQPENNYETTPPVLDSDGSFFL
YSKLTVDESRWQQGNVFSCSVMHEALHNHYTQKSLSLSPGK
Ig Kappa constant region (SEQ ID NO: 62):
          TVAAPSVFIFPPSDEQLKSGTASVVCLLNNFYPREAKVQWKVDN
ALQSGNSQESVTEQDSKDSTYSLSSTLTLSKADYEKHKVYACEVTHQGL
SSPVTKSFNRGEC
Ig Lambda constant region (SEQ ID NO: 63):
          QPKAAPSVTLFPPSSEELQANKATLVCLISDFYPGAVTVAWKAD
SSPVKAGVETTTPSKQSNNKYAASSYLSLTPEQWKSHRSYSCQVTHEGS
TVEKTVAPTEC
```

The term “antigen-binding portion” of an antibody refers to one or more fragments of an antibody that retain the ability to specifically bind to an antigen. The antigen-binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment, consisting of the VH and CH1 domains; (iv) a Fv fragment, consisting of the VL and VH domains of a single arm of an antibody; (v) a dAb fragment, consisting of a single variable domain; (vi) an isolated complementarity determining region (CDR), and (vii) an scFv, consisting of a single protein chain in which the VL and VH regions pair to form a monovalent molecule Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites. Such antibody binding portions are known in the art (Kontermann and Dubel eds., Antibody Engineering (2001) Springer-Verlag. New York. 790 pp. (ISBN 3-540-41354-5). In addition, single chain antibodies also include “linear antibodies” comprising a pair of tandem Fv segments (VH-CH1-VH-CH1) which, together with complementary light chain polypeptides, form a pair of antigen binding regions (Zapata et al. (1995) Protein Eng. 8(10):1057-1062; and U.S. Pat. No. 5,641,870).

The term “multivalent binding protein” means a binding protein comprising two or more antigen binding sites. In an embodiment, the multivalent binding protein is engineered to have the three or more antigen binding sites, and is generally not a naturally occurring antibody. The term “multispecific binding protein” refers to a binding protein capable of binding two or more related or unrelated targets. Dual variable domain (DVD) binding proteins provided herein comprise two or more antigen binding sites and are tetravalent or multivalent binding proteins. DVD binding proteins may be monospecific, i.e., capable of binding one antigen or multispecific, i.e., capable of binding two or more antigens. DVD-binding proteins comprising two heavy chain DVD polypeptides and two light chain DVD polypeptides are referred to as a DVD. Each half of a DVD binding protein comprises a heavy chain DVD polypeptide, and a light chain DVD polypeptide, and two antigen binding sites. Each binding site comprises a heavy chain variable domain and a light chain variable domain with a total of 6 CDRs involved in antigen binding per antigen binding site. Detailed description of specific DVD-binding protein molecules capable of binding specific targets (e.g., TNF and IL-17), and methods of making the same, is provided in the Examples section below and in U.S. Pat. No. 8,835,610, U.S. Pat. No. 8,779,101, U.S. patent publication number 20130164256, and U.S. application serial number 20140170152, U.S. application serial number 20140161804, and U.S. patent publication number 20140079705, each of which is incorporated herein in its entirety.

The term “bispecific antibody” refers to an antibody that binds one antigen (or epitope) on one of its two binding arms (one pair of HC/LC), and binds a different antigen (or epitope) on its second arm (a different pair of HC/LC). By this definition, a bispecific antibody has two distinct antigen binding arms (in both specificity and CDR sequences), and is monovalent for each antigen to which it binds.

The term “dual-specific antibody” refers to an antibody that can bind two different antigens (or epitopes) in each of its two binding arms (a pair of HC/LC). Accordingly a dual-specific binding protein has two identical antigen binding arms, with identical specificity and identical CDR sequences, and is bivalent for each antigen to which it binds.

The term “functional antigen binding site” means that the binding site of the binding protein is one that is capable of binding a target antigen.

The term “immunoglobulin constant domain” refers to a heavy or light chain constant domain. Human IgG heavy chain and light chain constant domain amino acid sequences are known in the art.

The term “monoclonal antibody” or “mAb” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method.

The term “human antibody” includes antibodies having variable and constant regions derived from human germline immunoglobulin sequences. The human antibodies provided herein may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody” does not include antibodies in which CDR sequences derived from the germline of another mammalian species, such as a mouse, have been grafted onto human framework sequences.

The term “recombinant human antibody” means human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. The term “CDR” means the complementarity determining region within antibody variable sequences. There are three CDRs in each of the variable regions of the heavy chain and the light chain, which are designated CDR1, CDR2, and CDR3, for each of the variable regions. The term “CDR set” means a group of three CDRs that occur in a single variable region (i.e., VH or VL) of an antigen binding site. The exact boundaries of these CDRs have been defined differently according to different systems. The system described by Kabat (Kabat et al. (1987, 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk (1987) J. Mol. Biol. 196: 901-917 and Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2, and L3 or H1, H2, and H3, where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139 and MacCallum (1996) J. Mol. Biol. 262(5): 732-745). Still other CDR boundary definitions may not strictly follow one of the above systems, but nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. A FR represents one of the four sub-regions, and FRs represents two or more of the four sub-regions constituting a framework region.

The terms “Kabat numbering”, “Kabat definitions” and “Kabat labeling” mean a system of numbering amino acid residues which are more variable (i.e., hypervariable) than other amino acid residues in the heavy and light chain variable regions of an antibody, or an antigen binding portion thereof (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region generally ranges from amino acid positions 31 to 35 for CDR1, amino acid positions 50 to 65 for CDR2, and amino acid positions 95 to 102 for CDR3. For the light chain variable region, the hypervariable region generally ranges from amino acid positions 24 to 34 for CDR1, amino acid positions 50 to 56 for CDR2, and amino acid positions 89 to 97 for CDR3.

The growth and analysis of extensive public databases of amino acid sequences of variable heavy and light regions over the past twenty years have led to the understanding of the typical boundaries between framework regions (FR) and CDR sequences within variable region sequences and enabled persons skilled in this art to accurately determine the CDRs according to Kabat numbering, Chothia numbering, or other systems. See, e.g., Martin, “Protein Sequence and Structure Analysis of Antibody Variable Domains, “In Kontermann and Dübel, eds., Antibody Engineering (Springer-Verlag, Berlin, 2001), chapter 31, pages 432-433. A useful method of determining the amino acid sequences of Kabat CDRs within the amino acid sequences of variable heavy (VH) and variable light (VL) regions is provided below:

To identify a CDR-L1 amino acid sequence:
Starts approximately 24 amino acid residues from the amino terminus of the VL region;
Residue before the CDR-L1 sequence is always cysteine (C);
Residue after the CDR-L1 sequence is always a tryptophan (W) residue, typically Trp-Tyr-Gln (W-Y-Q), but also Trp-Leu-Gln (W-L-Q), Trp-Phe-Gln (W-F-Q), and Trp-Tyr-Leu (W-Y-L);
Length is typically 10 to 17 amino acid residues.

To identify a CDR-L2 amino acid sequence:
Starts always 16 residues after the end of CDR-L1;
Residues before the CDR-L2 sequence are generally Ile-Tyr (I-Y), but also Val-Tyr (V-Y), Ile-Lys (I-K), and Ile-Phe (I-F);
Length is always 7 amino acid residues.

To identify a CDR-L3 amino acid sequence:
Starts always 33 amino acids after the end of CDR-L2;
Residue before the CDR-L3 amino acid sequence is always a cysteine (C);
Residues after the CDR-L3 sequence are always Phe-Gly-X-Gly (F-G-X-G) (SEQ ID NO:67), where X is any amino acid;
Length is typically 7 to 11 amino acid residues.

To identify a CDR-H1 amino acid sequence:
Starts approximately 31 amino acid residues from amino terminus of VH region and always 9 residues after a cysteine (C);
Residues before the CDR-H1 sequence are always Cys-X-X-X-X-X-X-X-X (SEQ ID NO:68), where X is any amino acid;
Residue after CDR-H1 sequence is always a Trp (W), typically Trp-Val (W-V), but also Trp-Ile (W-I), and Trp-Ala (W-A);
Length is typically 5 to 7 amino acid residues.

To identify a CDR-H2 amino acid sequence:
Starts always 15 amino acid residues after the end of CDR-H1;
Residues before CDR-H2 sequence are typically Leu-Glu-Trp-Ile-Gly (L-E-W-I-G) (SEQ ID NO:69), but other variations also;
Residues after CDR-H2 sequence are Lys/Arg-Leu/Ile/Val/Phe/Thr/Ala-Thr/Ser/Ile/Ala (K/R-L/I/V/F/T/A-T/S/I/A);
Length is typically 16 to 19 amino acid residues.

To identify a CDR-H3 amino acid sequence:
Starts always 33 amino acid residues after the end of CDR-H2 and always 3 after a cysteine (C)′
Residues before the CDR-H3 sequence are always Cys-X-X (C-X-X), where X is any amino acid, typically Cys-Ala-Arg (C-A-R);
Residues after the CDR-H3 sequence are always Trp-Gly-X-Gly (W-G-X-G) (SEQ ID NO:70), where X is any amino acid;
Length is typically 3 to 25 amino acid residues.

With respect to constructing DVD-Ig or other binding protein molecules, the term “linker” means a single amino acid or a polypeptide comprising two or more amino acid residues joined by peptide bonds (“linker polypeptide”) used to link one or more antigen binding portions. Such linker polypeptides are well known in the art (see, e.g., Holliger et al. (1993) Proc. Natl. Acad. Sci. USA 90: 6444-6448; Poljak (1994) Structure 2: 1121-1123). Exemplary linkers include, but are not limited to, GGGGSG (SEQ ID NO:24), GGSGG (SEQ ID NO:25), GGGGSGGGGS (SEQ ID NO:26), GGSGGGGSG (SEQ ID NO:27), GGSGGGGSGS (SEQ ID NO:28), GGSGGGGSGGGGS (SEQ ID NO:29), GGGGSGGGGSGGGG (SEQ ID NO:30), GGGGSGGGGSGGGGS (SEQ ID NO:31), ASTKGP (SEQ ID NO:32), ASTKGPSVFPLAP (SEQ ID NO:33), TVAAP (SEQ ID NO:34), RTVAAP (SEQ ID NO:35), TVAAPSVFIFPP (SEQ ID NO:36), RTVAAPSVFIFPP (SEQ ID NO:37), AKTTPKLEEGEFSEAR (SEQ ID NO:38), AKTTPKLEEGEFSEARV (SEQ ID NO:39), AKTTPKLGG (SEQ ID NO:40), SAKTTPKLGG (SEQ ID NO:41), SAKTTP (SEQ ID NO:42), RADAAP (SEQ ID NO:43), RADAAPTVS (SEQ ID NO:44), RADAAAAGGPGS (SEQ ID NO:45), RADAAAAGGGGSGGGGSGGGGSGGGGS (SEQ ID NO:46), SAKTTPKLEEGEFSEARV (SEQ ID NO:47), ADAAP (SEQ ID NO:48), ADAAPTVSIFPP (SEQ ID NO:49), QPKAAP (SEQ ID NO:50), QPKAAPSVTLFPP (SEQ ID NO:51), AKTTPP (SEQ ID NO:52), AKTTPPSVTPLAP (SEQ ID NO:53), AKTTAP (SEQ ID NO:54), AKTTAPSVYPLAP (SEQ ID NO:55), GENKVEYAPALMALS (SEQ ID NO:56), GPAKELTPLKEAKVS (SEQ ID NO:57), and GHEAAAVMQVQYPAS (SEQ ID NO:58).

The term “neutralizing” means to render inactive an activity, e.g., the biological activity of an antigen when a binding protein specifically binds the antigen. Preferably, a neutralizing binding protein described herein binds to human TNF-α and/or human IL-17 resulting in the inhibition of a biological activity of each of the cytokines. Preferably, the neutralizing binding protein binds TNF-α and IL-17 and reduces a biological activity of TNF-α and IL-17 by at least about 20%, 30%, 40%, 50%, 60%, 70%, 80%, 85%, 90%, 95%, or more Inhibition of a biological activity of TNF-α and IL-17 by a neutralizing binding protein can be assessed by measuring one or more indicators of TNF-α and IL-17 biological activity well known in the art.

The term “activity” includes activities such as the binding specificity/affinity of an antibody for an antigen, for example, a binding protein that binds to TNF-α and/or IL-17.

The term “epitope” means a polypeptide determinant capable of specific binding to an immunoglobulin or T-cell receptor. In certain embodiments, epitope determinants include chemically active surface groupings of molecules such as amino acids, sugar side chains, phosphoryl or sulfonyl groups, and, in certain embodiments, may have specific three dimensional structural characteristics and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. Antibodies are said to bind to the same epitope if the antibodies cross-compete (one prevents the binding or modulating effect of the other). In addition, structural definitions of epitopes (overlapping, similar, identical) are informative, but functional definitions are often more relevant as they encompass structural (binding) and functional (modulation, competition) parameters.

The term “percent identity” means a quantitative measurement of the similarity between two sequences (complete amino acid sequence or a portion thereof). Calculations of sequence identity between sequences are known by those in the art. For example, to determine the percent identity of two amino acid sequences, the sequences are aligned for optimal comparison purposes (e.g., gaps can be introduced in one or both of a first and a second amino acid sequence for optimal alignment). The amino acid residues at corresponding amino acid positions or nucleotide positions are then compared. When a position in the first sequence is occupied by the same amino acid residue or nucleotide as the corresponding position in the second sequence, then the proteins are identical at that position. The percent identity between the two sequences is a function of the number of identical positions shared by the sequences, taking into account the number of gaps, and the length of each gap, which need to be introduced for optimal alignment of the two sequences. For example, percent identity can about 30%, 35%, 40%, 45%, 50%, 55%, 60%, 65%, 70%, 75%, 80%, 85%, 90%, 95%, 96%, 98%, 99%, or 99% or more.

The comparison of sequences and determination of percent identity between two sequences are accomplished using a mathematical algorithm. Percent identity between two amino acid sequences is determined using an alignment software program using the default parameters. Suitable programs include, for example, CLUSTAL W (see Thompson et al. (1994) Nucl. Acids Res. 22: 4673-4680) or CLUSTAL X.

The term “substantially identical” in reference to amino acid sequences means a first amino acid sequence that contains a sufficient or minimum number of amino acid residues that are identical to aligned amino acid residues in a second amino acid sequence such that the first and second amino acid sequences can have a common structural domain and/or common functional activity. For example, amino acid sequences that contain a common structural domain having at least about 30%, 35%, 40%, 45%, 50%, 55%, 60%, 65%, 70%, 75%, 80%, 85%, 90%, 95%, 96%, 98%, 99%, or 99% or more identity to a DVD-Ig binding protein described herein (e.g., a DVD-Ig binding protein comprising SEQ ID NO: 4, SEQ ID NO: 9, SEQ ID NO: 11, SEQ ID NO: 16, or a portion or combination thereof). In various embodiments, the substantially identical protein includes an amino acid sequence that is at least about 30%, about 35%, about 40%, about 45%, about 50%, about 55%, about 60%, about 65%, about 70%, about 75%, about 80%, about 85%, about 90%, about 95%, about 99%, or 99% or more identical to SEQ ID NO: 4, SEQ ID NO: 9, or a portion or a combination thereof.

The terms “Kon,” “Kₒₙ” and “kon” mean the on rate constant for association or “association rate constant,” of a binding protein (e.g., an antibody) to an antigen to form an association complex, e.g., antibody/antigen complex, as is known in the art. The term “Kon” also is known by the terms “association rate constant” or “ka”. This value indicates the binding rate of an antibody to its target antigen or the rate of complex formation between an antibody and antigen as is shown by the equation below:

Antibody (“Ab”)+Antigen (“Ag”)→Ab-Ag

The terms “Koff,” “K_{off},” and “koff” mean the off rate constant for dissociation, or “dissociation rate constant,” of a binding protein (e.g., an antibody) from an association complex (e.g., an antibody/antigen complex) as is known in the art. This value indicates the dissociation rate of an antibody from its target antigen or separation of Ab-Ag complex over time into free antibody and antigen as shown by the equation below:

Ab+Ag←Ab-Ag

The terms “KD”, “Kd” and the “equilibrium dissociation constant,” mean the value obtained in a titration measurement at equilibrium, or by dividing the dissociation rate constant (Koff) by the association rate constant (Kon). The association rate constant (Kon), the dissociation rate constant (Koff), and the equilibrium dissociation constant (K are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used. Additionally, a KinExA® (Kinetic Exclusion Assay) assay, available from Sapidyne Instruments (Boise, Id.) can also be used.

The terms “AUC” and “area under the curve” mean the area under the plasma drug concentration-time curve and reflects the actual body exposure to drug after administration of a dose of the drug. AUC is typically related to clearance. A higher clearance rate is related to a smaller AUC, and a lower clearance rate is related to a larger AUC value. The AUC higher values represent slower clearance rates.

The term “volume of distribution” means the theoretical volume of fluid into which the total drug administered would have to be diluted to produce the concentration in plasma. Calculating the volume of distribution may in various embodiments involve the quantification of the distribution of a drug, e.g., a TNFα/IL-17 DVD-Ig binding protein, or antigen-binding portion thereof, between plasma and the rest of the body after dosing. The volume of distribution is the theoretical volume in which the total amount of drug would need to be uniformly distributed in order to produce the desired blood concentration of the drug.

The terms “half-life” and “T½” mean the time for half of a drug's concentration or activity (e.g., pharmacologic or physiologic) to be measurable compared to a previously measured peak concentration or activity. In various embodiments, the quantification of the half-life may involve determining the time taken for half of the concentration or activity a dose of a drug to be measurable, e.g., in the blood, or other body fluid, in a subject or same over time. For example, the half-life may involve the time taken for half of the dose to be eliminated, excreted or metabolized.

The term “Cmax” means the peak concentration that a drug is observed, quantified or measured in a specified fluid or sample after the drug has been administrated. In various embodiments, determining the Cmax involves in part quantification of the maximum or peak serum or plasma concentration of a drug/therapeutic agent observed in a sample from a subject administered the drug.

The term “bioavailability” means the degree to which a drug is absorbed or becomes available to cells or tissue after administration of the drug. For example, bioavailability in certain embodiments involves quantification of the fraction or percent of a dose which is absorbed and enters the systemic circulation after administration of a given dosage form. See international publication number WO2013078135, which is incorporated by reference herein in its entirety.

The terms “crystal” and “crystallized” mean an agent in the form of a crystal. Crystals are one form of the solid state of matter that is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. See Giegé et al., Chapter 1, In Crystallization of Nucleic Acids and Proteins, a Practical Approach, 2nd ed., (Ducruix and Giegé, eds.) (Oxford University Press, New York, 1999) pp. 1-16.

The term “polynucleotide” means a polymer of two or more nucleotides, e.g., ribonucleotides or deoxynucleotides or a modified form of nucleotide. The term includes single and double stranded forms of DNA.

The term “isolated polynucleotide” means a polynucleotide (e.g., of genomic, cDNA, or synthetic origin, or some combination thereof) that, by virtue of its origin, is not associated with all or a portion of a polynucleotide with which the polynucleotide is found in nature; is operably linked to a polynucleotide that it is not linked to in nature; or does not occur in nature as part of a larger sequence.

The terms “antagonist” and “inhibitor” mean a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of human TNF-α and IL-17. Antagonists and inhibitors of human TNF-α and IL-17 may include, but are not limited to, proteins, nucleic acids, carbohydrates, or any other molecules, which bind to human TNF-α and IL-17.

The term “effective amount” means the amount of a therapy that is sufficient to reduce or ameliorate the severity and/or duration of a disorder or one or more symptoms thereof; prevent the advancement of a disorder; cause regression of a disorder; prevent the recurrence, development, onset, or progression of one or more symptoms associated with a disorder; detect a disorder; or enhance or improve the prophylactic or therapeutic effect(s) of another therapy (e.g., prophylactic or therapeutic agent).

The terms “patient” and “subject” mean an animal, such as a mammal, including a primate (for example, a human, a monkey, and a chimpanzee), a non-primate (for example, a cow, a pig, a camel, a llama, a horse, a goat, a rabbit, a sheep, a hamster, a guinea pig, a cat, a dog, a rat, a mouse, a whale), a bird and a fish. In an embodiment, the patient or subject is a human, such as a human being treated or assessed for a disease, disorder or condition; a human at risk for a disease, disorder or condition; and/or a human having a disease, disorder or condition.

The term “sample” means a quantity of a substance. The term “biological sample means a quantity of a substance from a living thing or formerly living thing. Such substances include, but are not limited to, blood, plasma, serum, urine, amniotic fluid, synovial fluid, endothelial cells, leukocytes, monocytes, other cells, organs, tissues, bone marrow, lymph nodes and spleen.

The term “component” means a portion of a molecule, mixture, composition, system or kit, for example a capture antibody, a detection or conjugate antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), an analyte, a stop solution, and the like that can be included in a kit for assay of a test sample, such as a patient urine, serum or plasma sample, in accordance with the methods described herein and other methods known in the art. Some components can be in solution or lyophilized for reconstitution for use in an assay.

The term “control” means a component or composition that is not, or does not contain, an analyte (“negative control”) or is or contains analyte (“positive control”). A positive control can comprise a known concentration of analyte. A “calibrator” means a composition comprising a known concentration of analyte. A positive control can be used to establish assay performance characteristics and is a useful indicator of the integrity of reagents (e.g., analytes).

The term “risk” means the possibility or probability of a particular event occurring either presently or at some point in the future. The term “risk stratification” means an array of known clinical risk factors that allows physicians to classify patients into a low, moderate, high or highest risk of developing a particular disease, disorder or condition.

The terms “DMARD resistance” and “resistance to a DMARD” means an observed or demonstrated loss of efficacy over time to treatment of a disorder (e.g., RA) using a DMARD. DMARDs resistance may be a multifactorial event including enhanced drug efflux via ABC transporters, impaired drug uptake and drug activation, enhanced drug detoxification etc. In various embodiments, the subject is observed to have a RA symptom that is not reduced by DMARD treatment.

The term “a disorder in which antigen activity is detrimental” is intended to include diseases and other disorders in which the presence of the antigen in a subject suffering from the disorder has been shown to be or is suspected of being either responsible for the pathophysiology of the disorder or a factor that contributes to a worsening of the disorder. Accordingly, a disorder in which antigen activity is detrimental is a disorder in which reduction of antigen activity is expected to alleviate the symptoms and/or progression of the disorder. Such disorders may be evidenced, for example, by an increase in the concentration of the antigen in a biological fluid of a subject suffering from the disorder (e.g., an increase in the concentration of antigen in serum, plasma, synovial fluid, etc. of the subject). Non-limiting examples of disorders that can be treated with the binding proteins provided herein include those disorders discussed below and in the section pertaining to pharmaceutical compositions comprising the binding proteins.

Inflammatory Disorders

The binding proteins described herein can be used to treat rheumatoid arthritis (RA). In one embodiment, treatment encompasses reducing the severity of at least one symptom associated with a disease state. Symptoms associated with inflammatory disorders and/or arthritis include inflammation/swelling; stiffness, pain, hyperplasia, synovitis, fever, chills, joint inflammation, tenderness, loss of appetite, weight loss, and anemia and rash.

Pharmaceutical Compositions

Pharmaceutical compositions comprising a binding protein and a pharmaceutically acceptable carrier are provided. The pharmaceutical compositions comprising binding proteins provided herein are for use in, but not limited to, diagnosing, detecting, or monitoring a disorder, in preventing, treating, managing, or ameliorating of a disorder or one or more symptoms thereof, and/or in research. In a specific embodiment, a composition comprises one or more binding proteins provided herein. In another embodiment, the pharmaceutical composition comprises one or more binding proteins provided herein and one or more prophylactic or therapeutic agents other than binding proteins provided herein for treating a disorder. In an embodiment, the prophylactic or therapeutic agents known to be useful for or having been or currently being used in the prevention, treatment, management, or amelioration of a disorder or one or more symptoms thereof. In accordance with these embodiments, the composition may further comprise of a carrier, diluent or excipient.

The binding proteins provided herein can be incorporated into pharmaceutical compositions suitable for administration to a subject. Typically, the pharmaceutical composition comprises a binding protein provided herein and a pharmaceutically acceptable carrier. The term “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In some embodiments, isotonic agents, for example, sugars, polyalcohols such as mannitol, sorbitol, or sodium chloride, are included in the composition. Pharmaceutically acceptable carriers may further comprise minor amounts of auxiliary substances such as wetting or emulsifying agents, preservatives or buffers, which enhance the shelf life or effectiveness of the antibody or antibody binding portion.

Various delivery systems are known and can be used to administer one or more antibodies provided herein or the combination of one or more antibodies provided herein and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing the antibody or antibody fragment, receptor-mediated endocytosis, construction of a nucleic acid as part of a retroviral or other vector, etc. Methods of administering a prophylactic or therapeutic agent provided herein include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer, and formulation with an aerosolizing agent. See, e.g., U.S. Pat. No. 6,019,968. In one embodiment, a binding protein provided herein, combination therapy, or a composition provided herein is administered using Alkermes AIR® pulmonary drug delivery technology (Alkermes, Inc., Cambridge, Mass.). In a specific embodiment, prophylactic or therapeutic agents provided herein are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal mucosa, and intestinal mucosa, etc.) and may be administered together with other biologically active agents. Administration can be systemic or local.

In a specific embodiment, it may be desirable to administer the prophylactic or therapeutic agents provided herein locally to the area in need of treatment; this may be achieved by, for example, and not by way of limitation, local infusion, by injection, or by means of an implant, the implant being of a porous or non-porous material, including membranes and matrices, such as sialastic membranes, polymers, fibrous matrices (e.g., Tissuel®), or collagen matrices. In one embodiment, an effective amount of one or more antibodies provided herein antagonists is administered locally to the affected area to a subject to prevent, treat, manage, and/or ameliorate a disorder or a symptom thereof. In another embodiment, an effective amount of one or more antibodies provided herein is administered locally to the affected area in combination with an effective amount of one or more therapies (e.g., one or more prophylactic or therapeutic agents) other than a binding protein provided herein to a subject to prevent, treat, manage, and/or ameliorate a disorder or one or more symptoms thereof.

In another embodiment, the prophylactic or therapeutic agent can be delivered in a controlled release or sustained release system. In one embodiment, a pump may be used to achieve controlled or sustained release. In another embodiment, polymeric materials can be used to achieve controlled or sustained release of the therapies. In an embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose. Controlled release systems are discussed in the review by Langer (1990) Science 249:1527-1533). Any technique known to one of skill in the art can be used to produce sustained release formulations comprising one or more therapeutic agents provided herein.

In a specific embodiment, where the composition is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus. Alternatively, a nucleic acid can be introduced intracellularly and incorporated within host cell DNA for expression by homologous recombination.

A pharmaceutical composition provided herein is formulated to be compatible with its intended route of administration. Where useful, the composition may also include a solubilizing agent and a local anesthetic such as lignocaine to ease pain at the site of the injection.

The method may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

The methods provided herein may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods provided herein encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In one embodiment, one or more of the prophylactic or therapeutic agents, or pharmaceutical compositions provided herein is packaged in a hermetically sealed container such as an ampoule or sachette indicating the quantity of the agent. In one embodiment, one or more of the prophylactic or therapeutic agents, or pharmaceutical compositions provided herein is supplied as a dry sterilized lyophilized powder or water free concentrate in a hermetically sealed container and can be reconstituted (e.g., with water or saline) to the appropriate concentration for administration to a subject. In an embodiment, one or more of the prophylactic or therapeutic agents or pharmaceutical compositions provided herein is supplied as a dry sterile lyophilized powder in a hermetically sealed container at a unit dosage of at least 5 mg, at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions provided herein may be stored at between 2° C. and 8° C. in its original container and the prophylactic or therapeutic agents, or pharmaceutical compositions provided herein may be administered within 1 week, e.g., within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions provided herein is supplied in liquid form in a hermetically sealed container indicating the quantity and concentration of the agent. In an embodiment, the liquid form of the administered composition is supplied in a hermetically sealed container at least 0.25 mg/ml, at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form may be stored at between 2° C. and 8° C. in its original container.

The binding proteins provided herein can be incorporated into a pharmaceutical composition suitable for parenteral administration. In an embodiment, the binding protein is prepared as an injectable solution. For example, the solution contains 0.1-250 mg/mL of the binding protein. The injectable solution can be composed of either a liquid or lyophilized dosage form in a flint or amber vial, ampule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants. The pharmaceutical composition comprising the binding proteins provided herein prepared as an injectable solution for parenteral administration, can further comprise an agent useful as an adjuvant, such as those used to increase the absorption, or dispersion of a therapeutic protein (e.g., antibody). A particularly useful adjuvant is hyaluronidase, such as Hylenex® (recombinant human hyaluronidase). Addition of hyaluronidase in the injectable solution improves human bioavailability following parenteral administration, particularly subcutaneous administration. It also allows for greater injection site volumes (i.e., greater than 1 ml) with less pain and discomfort, and minimum incidence of injection site reactions. (see PCT Publication No. WO2004078140 and U.S. Publication No. 2006104968).

The compositions provided herein may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form chosen depends on the intended mode of administration and therapeutic application. Typical compositions are in the form of injectable or infusible solutions, such as compositions similar to those used for passive immunization of humans with other antibodies. The chosen mode of administration is parenteral (e.g., intravenous, subcutaneous, intraperitoneal, intramuscular). In an embodiment, the antibody is administered by intravenous infusion or injection. In another embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody binding portion) in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated herein. In the case of sterile, lyophilized powders for the preparation of sterile injectable solutions, the methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The binding proteins provided herein can be administered by a variety of methods known in the art, although for many therapeutic applications, in an embodiment, the route/mode of administration is subcutaneous injection, intravenous injection or infusion. As is appreciated by the skilled artisan, the route and/or mode of administration varies depending upon the desired results. In certain embodiments, the active compound may be prepared with a carrier that protects the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Supplementary active compounds can also be incorporated into the compositions. In certain embodiments, a binding protein provided herein is coformulated with and/or coadministered with one or more additional therapeutic agents that are useful for treating disorders with a binding protein provided herein. For example, a binding protein provided herein may be coformulated and/or coadministered with one or more additional antibodies that bind other targets (e.g., antibodies that bind other cytokines or that bind cell surface molecules). Furthermore, one or more antibodies provided herein may be used in combination with two or more of the foregoing therapeutic agents. Such combination therapies may advantageously utilize lower dosages of the administered therapeutic agents, thus avoiding possible toxicities or complications associated with the various monotherapies.

In certain embodiments, a binding protein is linked to a half-life extending vehicle known in the art. Such vehicles include, but are not limited to, the Fc domain, polyethylene glycol, and dextran. Such vehicles are described, e.g., in U.S. Pat. No. 6,660,843 and PCT Publication No. WO1999/25044.

In a specific embodiment, nucleic acid sequences encoding a binding protein provided herein or another prophylactic or therapeutic agent provided herein are administered to treat, prevent, manage, or ameliorate a disorder or one or more symptoms thereof by way of gene therapy. Gene therapy refers to therapy performed by the administration to a subject of an expressed or expressible nucleic acid. In this embodiment, the nucleic acids produce their encoded antibody or prophylactic or therapeutic agent provided herein that mediates a prophylactic or therapeutic effect.

Any of the methods for gene therapy available in the art can be used in the methods provided herein. For general reviews of the methods of gene therapy. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990). Detailed description of various methods of gene therapy are disclosed in U.S. Patent Publication No. US20050042664.

A method for treating a human subject suffering from a disorder in which the target, or targets, capable of being bound by the binding protein disclosed herein is detrimental, comprising administering to the human subject a binding protein disclosed herein such that the activity of the target, or targets in the human subject is inhibited and one of more symptoms is alleviated or treatment is achieved is provided. In an embodiment, diseases that can be treated or diagnosed with the compositions and methods include, but are not limited to, immune and inflammatory elements, such as autoimmune diseases such as RA.

A binding protein provided herein also can be administered with one or more additional therapeutic agents useful in the treatment of various diseases.

A binding protein provided herein can be used alone or in combination to treat such diseases. It should be understood that the binding proteins can be used alone or in combination with an additional agent, e.g., a therapeutic agent, the additional agent being selected by the skilled artisan for its intended purpose. For example, the additional agent can be a therapeutic agent art-recognized as being useful to treat the disease or condition being treated by the antibody provided herein. The additional agent also can be an agent that imparts a beneficial attribute to the therapeutic composition e.g., an agent which effects the viscosity of the composition.

It should further be understood that the combinations provided herein are those combinations useful for their intended purpose. The agents set forth below are illustrative for purposes and not intended to be limited. In some embodiments, the combinations comprise the antibodies provided herein and at least one additional agent selected from the lists below. The combination can also include more than one additional agent, e.g., two or three additional agents if the combination is such that the formed composition can perform its intended function.

The pharmaceutical compositions provided herein may include a “therapeutically effective amount” or a “prophylactically effective amount” of a binding protein provided herein. A “therapeutically effective amount” refers to an amount effective, at dosages and for periods of time necessary, to achieve the desired therapeutic result. A therapeutically effective amount of the binding protein may be determined by a person skilled in the art and may vary according to factors such as the disease state, age, sex, and weight of the subject, and the ability of the binding protein to elicit a desired response in the subject. A therapeutically effective amount is also one in which any toxic or detrimental effects of the antibody, or antibody binding portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount is less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. The term “dosage unit form” means physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms provided herein are dictated by and directly dependent on (a) the unique characteristics of the active compound and the particular therapeutic or prophylactic effect to be achieved, and (b) the limitations inherent in the art of compounding such an active compound for the treatment of sensitivity in subjects.

An exemplary, non-limiting range for a therapeutically or prophylactically effective amount of a binding protein provided herein is 0.1-20 mg/kg, for example, 1-10 mg/kg. It is to be noted that dosage values may vary with the type and severity of the condition to be alleviated. It is to be further understood that for any particular subject, specific dosage regimens may be adjusted over time according to the subject need and the professional judgment of the person administering or supervising the administration of the compositions, and that dosage ranges set forth herein are exemplary only and are not intended to limit the scope or practice of the claimed composition.

### EXAMPLES

### Abbreviations

### ABBIOS Biosignal System

### ACR American College of Rheumatology

ADA Anti-drug antibody
AE Adverse event
ALT Alanine aminotransferase
AST Aspartate aminotransferase
Anti-CCP Anti-cyclic citrullinated peptide
aPTT Activated Partial Thromboplastin Time

### BL Baseline

BMI Body mass index
CBC Complete blood count
CPK Creatine phosphokinase
CVA Cerebral vascular accident
DAS28 Disease activity score 28
DM Diabetes mellitus
eCRF Electronic case report form
EDC Electronic data capture
EOW Every other week

### EP European Pharmacopoeia

ESR Erythrocyte sedimentation rate

### EudraCT European Union Drug Regulatory Authority Clinical Trials

### EULAR European League Against Rheumatism

### FOI Fluorescence Optical Imaging

FPG Fasting plasma glucose

### GCP Good Clinical Practice

### HAQ-DI Health Assessment Questionnaire Disability Index

### Hb Hemoglobin

HBsAg Hepatitis B surface antigen
HCV Ab Hepatitis C virus antibody
hsCRP High sensitivity C reactive protein

### IB Investigator Brochure

ICF Informed consent form

### ICH International Conference on Harmonization

### IEC Independent Ethics Committee

INR International normalized ratio

### IRB Institutional Review Board

LDH Lactate dehydrogenase
MCH Mean corpuscular hemoglobin
MCHC Mean corpuscular hemoglobin concentration
MCV Mean corpuscular volume

### MedDRA Medical Dictionary for Regulatory Activities

### NCI CTCAE National Cancer Institute Common Terminology Criteria for Adverse Events

NOAEL No observed adverse effect level

### NYHA New York Heart Association

### PD Premature Discontinuation

PDR Post dose reaction

### PG Pharmacogenetic

POR Proof of receipt
PT Prothrombin time
QTc QT interval corrected for heart rate
RF Rheumatoid factor
SAD Single ascending dose

### SAE Serious Adverse Event

### SCR Screening

SGPT/ALT Serum glutamic-pyruvic transaminase
SGOT/AST Serum glutamic-oxaloacetic transaminase
SJC Swollen joint count

### SUSAR Suspected Unexpected Serious Adverse Reaction

### TB Tuberculosis

TJC Tender joint count
TNF Tumor necrosis factor
VAS Visual analog scale
ULN Upper limit of normal

### Pharmacokinetic and Statistical Abbreviations

ANOVA Analysis of variance
ANCOVA Analysis of covariance
AUC Area under the serum concentration-time curve
AUCₜₐᵤ Area under the serum concentration-time curve over a dosing interval
AUC_{∞} Area under the serum concentration-time curve from time zero to infinity
β Terminal phase elimination rate constant

### CL Clearance

CL/F Apparent oral clearance
Cₘₐₓ Maximum observed serum concentration
C_{trough} Observed serum concentration prior to dose
t_{1/2} Terminal phase elimination half-life
Tₘₐₓ Time to maximum observed serum concentration
V Volume of distribution
V/F Apparent volume of distribution with subcutaneous distribution

### EXEMPLIFICATION

### Example 1

### Dual Neutralization of TNF and IL-17 with a DVD-Ig Protein in a Collagen Induced Arthritis (CIA) Model

To determine the potential therapeutic benefit of neutralization of TNF and IL-17 either alone or together, the efficacy of surrogate anti-mouse TNF and anti-mouse IL-17A antibodies, alone and in combination, was determined in the mouse collagen induced arthritis (CIA) model, a pre-clinical model of RA characterized by swelling, inflammation, and destruction of the joints (Bardwell et al. (2009) J. Immunol. 182(12):7482-7489). Multiple endpoints were assessed including clinical signs as measured by MAS. This measurement is a composite score based on the redness, swelling, and ankylosis of each paw (3-point scale for each paw with a maximum score of 12). In addition, the impact of bone erosions was evaluated by micro-computed tomography (CT) analysis of the ankle joint. Inflammation, cartilage, and bone loss within the affected joints were also evaluated histologically.

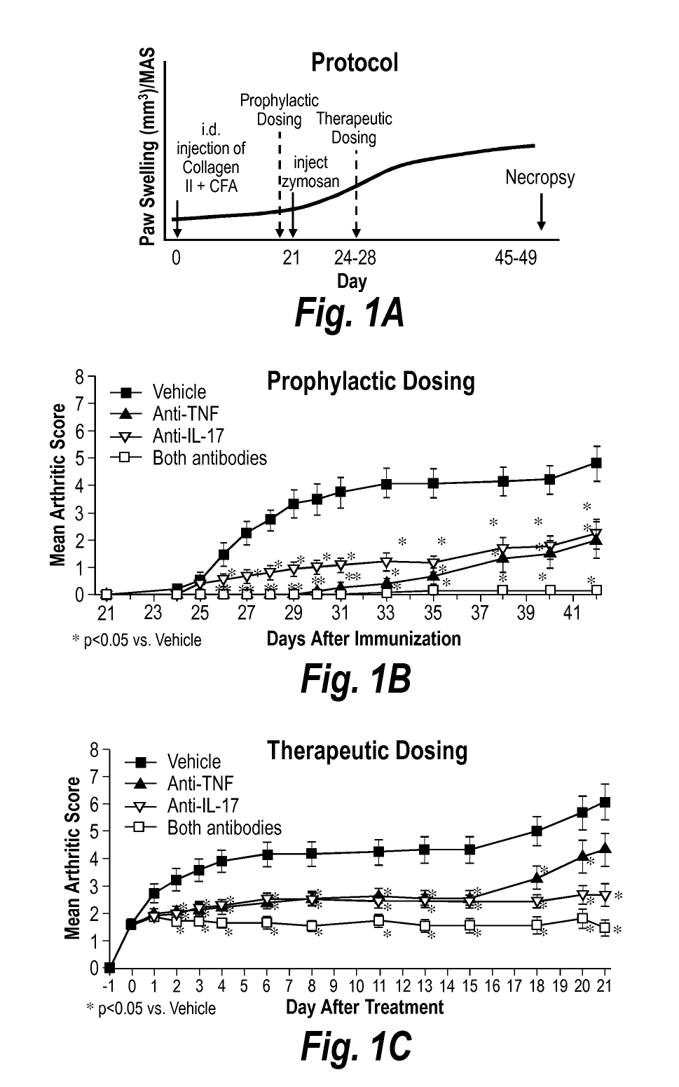

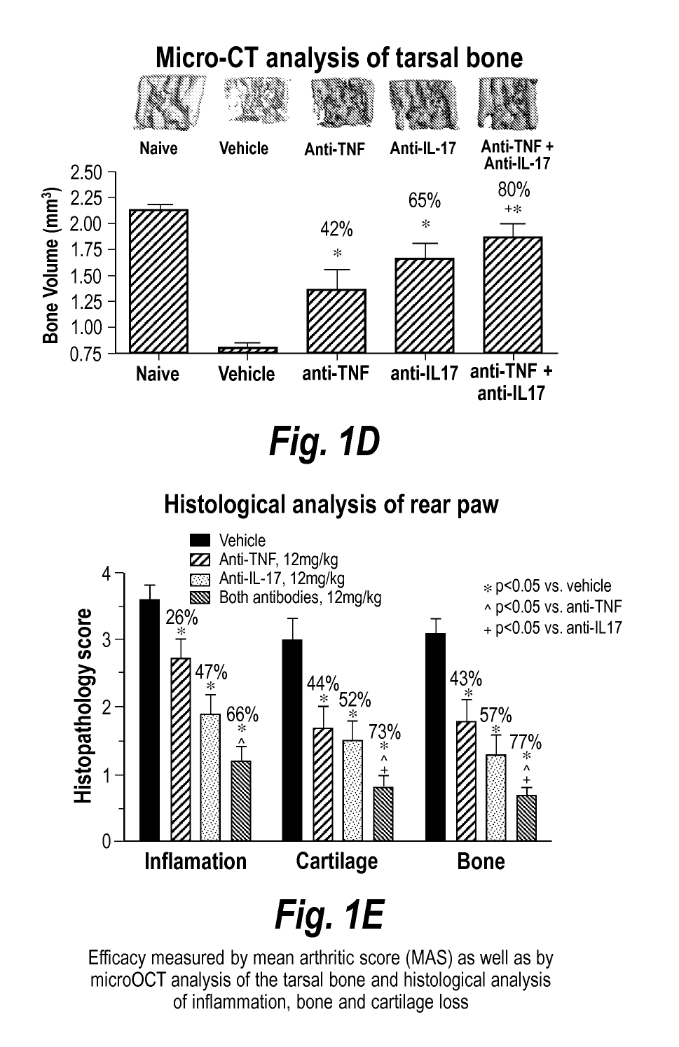

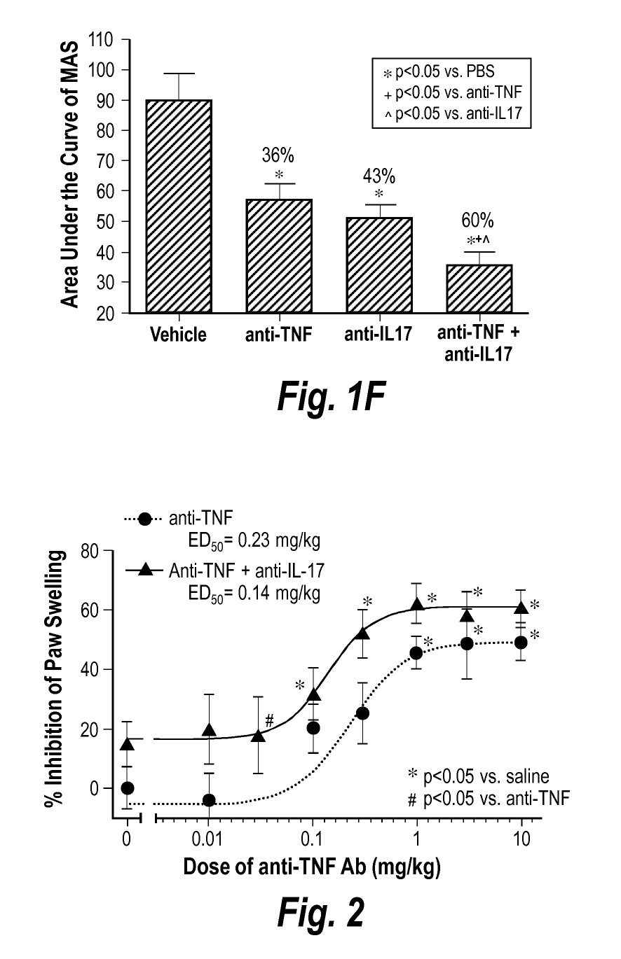

The therapeutic effects of an anti-IL-17 antibody, an anti-TNF antibody, and combined mixtures of anti-IL-17 antibodies/anti-TNF antibodies were evaluated in the CIA mouse model described herein (See FIG. 1 panel A). Briefly, male DBA-1 mice were immunized SC with bovine type II collagen in complete Freund's adjuvant (CFA) at the base of the tail and provided either a prophylactic dose 1 day prior to an intra-peritoneal injection of zymosan 21 days later, or were provided a therapeutic dose 3-7 days after the zymosan injection. The doses contained either anti-murine TNF antibody 8C11, anti-IL-17 antibody 10F7M11, or both anti-TNF antibody and anti-IL-17 antibodies. Sequences for the 8C11 antibody and the 10F7M11 antibody are shown in Tables 1-3. Control subjects were administered vehicle only.

TABLE 2
Sequences of 8C11 Antibody Variable Domains and CDRs

| | | | 1234567890123456789012345678901234 |
|---|---|---|---|
| | Identifier | Chain | 567890 |
| SEQ ID | 8C11-VH | VH | EFQLQQSGPELVKPGASVRISCKASGYSFTDYNM |
| NO: 1 | | | NWVKQSNGKSLEWVGVINPNYGSSTYNQKFKGKATLTVDQ |
| | | | SSSTAYMQLNSLTSEDSAVYYCARKWGQLGRGFFDVWGTG |
| | | | TTVTVSS |
| SEQ ID | 8C11-VL | VL | QIVLSQSPAILSASPGEKVTMTCRASSSVSYMHW |
| NO: 2 | | | FQQKPGSSPKPWIYATSNLASGVPARFSGSGSGTSYSLTI |
| | | | SRVEAEDAATYYCQQWSSSPLTFGAGTKLELKR |

| | |
|---|---|
| VH 8C11 CDR Set | |
| VH 8C11 CDR-H1 | Residues 31-35 of SEQ ID NO: 1 |
| VH 8C11 CDR-H2 | Residues 50-66 of SEQ ID NO: 1 |
| VH 8C11 CDR-H3 | Residues 99-109 of SEQ ID NO: 1 |
| VH 8C11 CDR Set | |

| | |
|---|---|
| VL 8C11 CDR-L1 | Residues 24-33 of SEQ ID NO: 2 |
| VL 8C11 CDR-L2 | Residues 49-55 of SEQ ID NO: 2 |
| VL 8C11 CDR-L3 | Residues 88-96 of SEQ ID NO: 2 |

TABLE 3
Sequences of 10F7M11 Antibody Variable Domains and CDRs

| | | | |
|---|---|---|---|
| SEQ ID | 10F7M11-VH | VH | QVQLQQSGAELVRPGTSVTLSCKASGYIFTDYEI |
| NO: 3 | | | HWVKQTPVHGLEWIGVNDPESGGTFYNQKFDGKAELTADK |
| | | | SSSTAYMELRSLTSEDSGVYYCTRYYRYESFYGNDYWGQG |
| | | | TSITVSS |
| SEQ ID | 10F7M11-VL | VL | QIVLTQSPAIMSASPGEKVTMTCSASSSISYIYW |
| NO: 4 | | | FQQKPGTSPKRWIYATFELASGVPARFSGSGSGTSYSLTI |
| | | | SSMEAEDAATYYCHQRSSYPWTFGGGSKLEIKR |

| | |
|---|---|
| VH 10F7M11 CDR Set | |
| VH 10F7M11 CDR-H1 | Residues 31-35 of SEQ ID NO: 3 |
| VH 10F7M11 CDR-H2 | Residues 50-66 of SEQ ID NO: 3 |
| VH 10F7M11 CDR-H3 | Residues 99-110 of SEQ ID NO: 3 |
| VH 10F7M11 CDR Set | |
| VL 10F7M11 CDR-L1 | Residues 24-33 of SEQ ID NO: 4 |
| VL 10F7M11 CDR-L2 | Residues 49-55 of SEQ ID NO: 4 |
| VL 10F7M11 CDR-L3 | Residues 88-96 of SEQ ID NO: 4 |

Mean arthritis score data (FIG. 1, panel B and FIG. 1, panel C), micro-CT analysis of tarsal bones (FIG. 1, panel D; and FIG. 1, panel E), and histological analysis of a rear paw from subjects show that treatment with both anti-TNF antibodies and anti-IL-17 antibodies was more efficacious than using either antibody alone. These data illustrate that interactions between TNF and IL-17 drive the arthritic process and further support the potential benefit of neutralizing both cytokines in RA.

Neutralization of both TNF and IL-17A inhibited bone loss to a greater extent than achieved with anti-TNF treatment alone (FIG. 1, panel F). Histologic evaluation confirmed the greater protection from bone loss and also demonstrated that concomitant neutralization of TNF and IL-17A reduced the amount of inflammation and cartilage destruction within the joint. Neutralization of TNF or IL-17A alone with anti-mouse Abs resulted in a partial inhibition of arthritic score (36% and 43% inhibition, respectively, P<0.05). However, when the 2 cytokines were neutralized by administration of anti-TNF and anti-IL-17A Abs, arthritic score was reduced to a greater extent (60%; P<0.05; FIG. 1 panels A-F). These data illustrate that interactions between TNF and IL-17 drive the arthritic process and further support the potential benefit of neutralizing both cytokines in RA.

To further understand how neutralization of IL-17 may impact the dose of TNF required for maximal efficacy, the dose response of the anti-TNF Ab in the presence or absence of a fixed dose of the anti-IL-17 Ab was determined in a shortened 7-day model of CIA with percent inhibition of paw swelling as the endpoint. In these studies, neutralization of IL-17A with a fixed dose of 6 mg/kg of the surrogate mouse anti-IL-17 Ab reduced the dose of anti-TNF required for maximal efficacy, from 1.0 to 0.3 mg/kg (FIG. 2). These data indicated the cooperative interaction of these cytokines in arthritic disease.

Without being limited by any particular theory or mechanism of action, it is here envisioned that subjects with RA may benefit from treatment with an agent that neutralizes both TNF and IL-17.

### Example 2

### Dual Neutralization of TNF and IL-17 with a DVD-Ig Protein in a Collagen Induced Arthritis Model

An anti-mouse TNF/IL-17 Dual Variable Domain Immunoglobulin (DVD-Ig) protein was employed in a mouse CIA model to determine whether dual neutralization of TNF and IL-17 with a bispecific molecule utilizing DVD-Ig technology would confer efficacy in an arthritis model with the intended pharmacologic activity in the joint.

TABLE 4
Sequences of Murine 8C11/10F7M11 DVD-Ig Binding
Proteins

| | | |
|---|---|---|
| DVD HEAVY | SEQ ID NO.: 5 | EFQLQQSGPELVKP |
| VARIABLE | | GASVRISCKASGYSFTDYN |
| 8C11-linker- | | MNWVKQSNGKSLEWVGV |
| 10F7M11-DVD | | INPNYGSSTYNQKFKGKAT |
| | | LTVDQSSSTAYMQLNSLTS |
| | | EDSAVYYCARKWGQLGR |
| | | GFFDVVVGTGTTVTVSSGG |
| | | GGSGGGGSQVQLQQSGAE |
| | | LVRPGTSVTLSCKASGYIF |
| | | TDYEIHWVKQTPVHGLEW |
| | | IGVNDPESGGTFYNQKFDG |
| | | KAELTADKSSSTAYMELRS |
| | | LTSEDSGVYYCTRYYRYES |
| | | FYGMDYVVGQGTSITVSS |
| 8C11 VH | SEQ ID NO.: 1 | EFQLQQSGPELVKP |
| | | GASVRISCKASGYSFTDYN |
| | | MNWVKQSNGKSLEWVGV |
| | | INPNYGSSTYNQKFKGKAT |
| | | LTVDQSSSTAYMQLNSLTS |
| | | EDSAVYYCARKWGQLGR |
| | | GFFDVVVGTGTTVTVSS |
| linker | SEQ ID NO.: 6 | GGGGSGGGGS |
| 10F7M11 VH | SEQ ID NO.: 3 | QVQLQQSGAELVR |
| | | PGTSVTLSCKASGYIFTDY |
| | | EIHWVKQTPVHGLEWIGV |
| | | NDPESGGTFYNQKFDGKA |
| | | ELTADKSSSTAYMELRSLT |
| | | SEDSGVYYCTRYYRYESFY |
| | | GMDYWGQGTSITVSS |
| CH | SEQ ID NO.: 7 | ASTKGPSVFPLAPS |
| | | SKSTSGGTAALGCLVKDY |
| | | FPEPVTVSWNSGALTSGVH |
| | | TFPAVLQSSGLYSLSSVVT |
| | | VPSSSLGTQTYICNVNHKP |
| | | SNTKVDKKVEPKSCDKTH |
| | | TCPPCPAPEAAGGPSVFLF |
| | | PPKPKDTLMISRTPEVTCV |
| | | VVDVSHEDPEVKFNWYVD |
| | | GVEVHNAKTKPREEQYNS |
| | | TYRVVSVLTVLHQDWLNG |
| | | KEYKCKVSNKALPAPIEKT |
| | | ISKAKGQPREPQVYTLPPS |
| | | REEMTKNQVSLTCLVKGF |
| | | YPSDIAVEWESNGQPENNY |
| | | KTTPPVLDSDGSFFLYSKL |
| | | TVDKSRWQQGNVFSCSVM |
| | | HEALHNHYTQKSLSLSPGK |
| DVD LIGHT | SEQ ID NO.: 8 | QIVLSQSPAILSASP |
| VARIABLE | | GEKVTMTCRASSSVSYMH |
| 8C11-linker- | | WFQQKPGSSPKPWIYATS |
| 10F7M11- | | NLASGVPARFSGSGSGTSY |
| DVD | | SLTISRVEAEDAATYYCQQ |
| | | WSSSPLTFGAGTKLELKR |
| | | GGSGGGGSGQIVLTQSPAI |
| | | MSASPGEKVTMTCSASSSI |
| | | SYIYWFQQKPGTSPKRWIY |
| | | ATFELASGVPARFSGSGSG |
| | | TSYSLTISSMEAEDAATYY |
| | | CHQRSSYPWTFGGGSKLEI |
| | | KR |
| 8C11 VL | SEQ ID NO.: 2 | QIVLSQSPAILSASP |
| | | GEKVTMTCRASSSVSYMH |
| | | WFQQKPGSSPKPWIYATS |
| | | NLASGVPARFSGSGSGTSY |
| | | SLTISRVEAEDAATYYCQQ |
| | | WSSSPLTFGAGTKLELKR |
| linker | SEQ ID NO.: 9 | GGSGGGGSG |
| 10F7M11 VL | SEQ ID NO.: 4 | QIVLTQSPAIMSASP |
| | | GEKVTMTCSASSSISYIYVV |
| | | FQQKPGTSPKRWIYATFEL |
| | | ASGVPARFSGSGSGTSYSL |
| | | TISSMEAEDAATYYCHQRS |
| | | SYPWTFGGGSKLEIKR |
| CL | SEQ ID NO.: 10 | TVAAPSVFIFPPSDE |
| | | QLKSGTASVVCLLNNFYPR |
| | | EAKVQWKVDNALQSGNS |
| | | QESVTEQDSKDSTYSLSST |
| | | LTLSKADYEKHKVYACEV |
| | | THQGLSSPVTKSFNRGEC |

### Mouse CIA Model

Disease was induced in male DBA/1J mice as described in Bardwell et al. (2009) J. Immunol. 182(12):7482-7489. Mice were immunized at the base of the tail with emulsion of type II bovine collagen in complete Freund's adjuvant. Mice were boosted 21 days later by i p administration of 1 mg zymosan A in phosphate buffered saline (PBS). Disease onset occurred within three to seven days following the zymosan challenge. Paw swelling was measured with calipers. Mice were treated at first clinical signs of disease with the anti-cytokine antibodies or 8C11/10F7 DVD-Ig protein described herein (Tables 4-5) twice a week by i.p. injection.

TABLE 5
Source And Binding Information Regarding Anti-TNF 8C11 Antibody,
Anti-IL-17 10F7 Antibody And the 8C11/10F7 DVD-Ig Protein

| | | | | | Dose (mg/kg) | |
|---|---|---|---|---|---|---|
| | | | IC₅₀ (nM) | | 21 day | 7 day |
| | Clone name | Isotype | IL-17 | TNF | CIA | CIA |
| Anti-TNF | 8C11 | Mouse | | 2 | 12 | 6 |
| | | IgG2c | | | | |
| Anti-IL-17 | MAB421 | Rat | 0.7 | 2 | 12 | 6 |
| | | IgG2a | | | | |
| Anti- | 8C11/10F7M11 | Mouse | 0.13 | 2 | 16 | 0.1-10 |
| TNF/IL-17 | | IgG2a | | | | |
| DVD-Ig | | | | | | |
| protein | | | | | | |

### Micro-CT Analysis of Bone Volume

Subject paws were imaged using a Scanco μCT40 (Scanco Medical AG) at 55 peak kilovoltage (kVp) and 145 microamperes (μA). A cylindrical contour was manually drawn around a region of interest from the proximal junction of the calcaneous and navicular bones and extending into the tarsals for a fixed height of 100 slices (1.8 mm) Three-dimensional (3D) quantitative evaluation was performed by ScancoAG analytical software for bone volume (mm³) and surface area to volumetric ratio, giving an approximation of tarsal surface roughness (mm¹)

### Analysis of Paw Homogenate

Paws were collected from all animals at the end of the study and stored at −80° C. Liquid nitrogen frozen paws were pulverized with a Bio-Pulverizer unit (BioSpec Products, Inc.) and homogenized in Radio-Immunoprecipitation Assay (RIPA) buffer using a bullet blender. Once homogenized, tubes were spun for 10 minutes at 10,000 RPM and the supernatants transferred to the assay plates. Both serum and paw homogenates were analyzed with Milliplex Map Mouse selected cytokine/chemokine magnetic panel bead system (Millipore) and the concentrations for all analytes were derived from Bio-Plex System fluorescence values (Biorad).

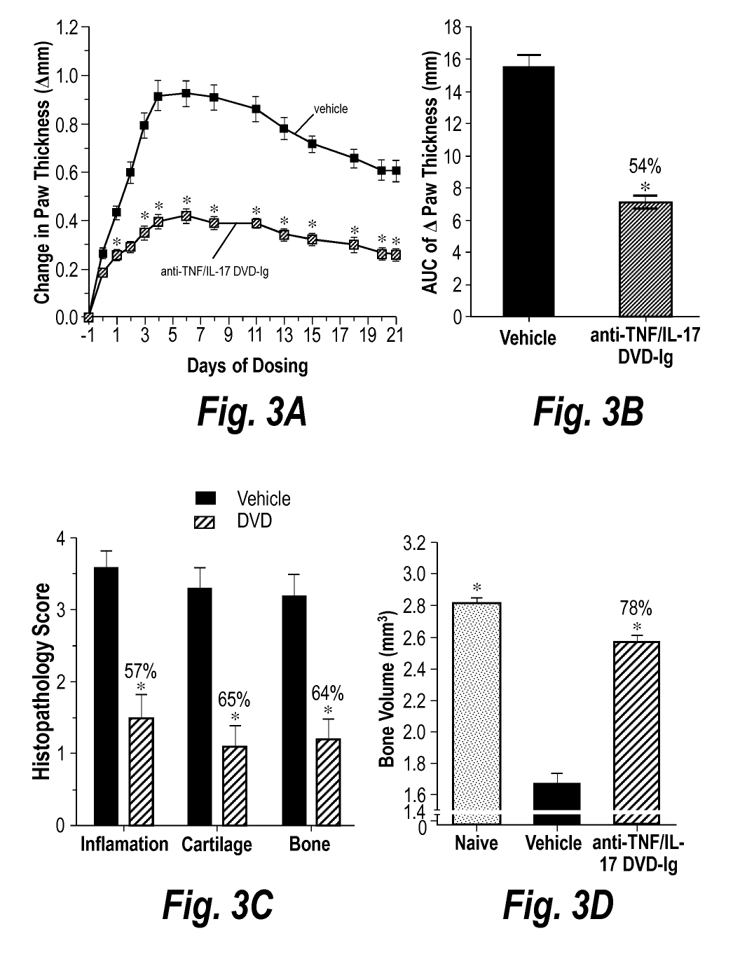

Data showed that the 8C11/10F7M11 anti-TNF/IL-17 DVD-Ig protein treatment was efficacious through 21 days in the mouse CIA model of rheumatoid arthritis. The 8C11/10F7M11 anti-TNF/IL-17 DVD-Ig protein inhibited paw swelling (FIG. 3, panel A and FIG. 3, panel B), reduced histological inflammation in and around the cartilage and bone (FIG. 3, panel C), and reduced the bone volume (FIG. 3, panel D).

In summary, the dual blockade and neutralization of TNF and IL-17 with a DVD-Ig protein is efficacious in a preclinical model of arthritis. Data herein support further clinical evaluation of other bispecific proteins, for example anti-human TNF/IL-17 bispecifics, such as DVD-Ig proteins, for the treatment of rheumatoid arthritis and other inflammatory diseases.

### Example 3

### Physical, Chemical, and Pharmaceutical Properties and Formulation of Anti Human TNF/IL-17 DVD-Ig ABBV-257

The dual binding and/or neutralization of TNF and IL-17 may provide superior efficacy to the current standard of care treatments for autoimmune and inflammatory diseases. Table 6 provides the amino acid sequences for ABBV-257, an anti-human TNF and IL-17 DVD-Ig binding protein having heavy chain and light chain domains comprising humanized and affinity matured variable domain sequences from parental mouse anti-TNF and anti-IL-17 antibodies.

TABLE 6
Heavy Variable Domain And Light Variable Domain
Amino Acid Sequences Of Anti-IL-17/TNF DVD-Ig
Protein, ABBV-257

| | | |
|---|---|---|
| DVD HEAVY | SEQ ID NO.: 11 | EVQLVQSGAEVKKPGASVKV |
| VARIABLE | | SCKASGYTFANYGIIWVRQA |
| HMAK199-1- | | PGQGLEWMGWINTYTGKPTY |
| GS10- | | AQKFQGRVTMTTDTSTSTAY |
| H10F7-M11 DVD | | MELSSLRSEDTAVYYCARKL |
| | | FTTMDVTDNAMDYWGQGTTV |
| | | TVSSGGGGSGGGGSEVQLVQ |
| | | SGAEVKKPGSSVKVSCKASG |
| | | YTFTDYEIHWVRQAPGQGLE |
| | | WMGVNDPESGGTFYNQKFDG |
| | | RVTLTADESTSTAYMELSSL |
| | | RSEDTAVYYCTRYSKWDSFD |
| | | GMDYWGQGTTVTVSS |
| HMAK199-1VH | SEQ ID NO.: 12 | EVQLVQSGAEVKKPGASVKV |
| | | SCKASGYTFANYGIIWVRQA |
| | | PGQGLEWMGWINTYTGKPTY |
| | | AQKFQGRVTMTTDTSTSTAY |
| | | MELSSLRSEDTAVYYCARKL |
| | | FTTMDVTDNAMDYWGQGTTV |
| | | TVSS |
| LINKER | SEQ ID NO.: 13 | GGGGSGGGGS |
| H10F7-M11 VH | SEQ ID NO.: 14 | EVQLVQSGAEVKKPGSSVKV |
| | | SCKASGYTFTDYEIHWVRQA |
| | | PGQGLEWMGVNDPESGGTFY |
| | | NQKFDGRVTLTADESTSTAY |
| | | MELSSLRSEDTAVYYCTRYS |
| | | KWDSFDGMDYWGQGTTVTVS |
| | | S |
| CH CG1234, | SEQ ID NO.: 15 | ASTKGPSVFPLAPSSKSTSG |
| 235 | | GTAALGCLVKDYFPEPVTVS |
| MUT Z NONA | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| DVD LIGHT | SEQ ID NO.: 16 | DIQMTQSPSSLSASVGDRVT |
| VARIABLE | | ITCRASQDISQYLNWYQQKP |
| HMAK199-1- | | GKAPKLLIYYTSRLQSGVPS |
| GS10- | | RFSGSGSGTDFTLTISSLQP |
| H10F7-M11DVD | | EDFATYFCQQGNTWPPTFGQ |
| | | GTKLEIKRGGSGGGGSGDIQ |
| | | MTQSPSSLSASVGDRVTITC |
| | | RASSGIISYIDWFQQKPGKA |
| | | PKRLIYATFDLASGVPSRFS |
| | | GSGSGTDYTLTISSLQPEDF |
| | | ATYYCRQVGSYPETFGQGTK |
| | | LEIKR |
| HMAK199-1 VL | SEQ ID NO.: 17 | DIQMTQSPSSLSASVGDRVT |
| | | ITCRASQDISQYLNWYQQKP |
| | | GKAPKLLIYYTSRLQSGVPS |
| | | RFSGSGSGTDFTLTISSLQP |
| | | EDFATYFCQQGNTWPPTFGQ |
| | | GTKLEIKR |
| LINKER | SEQ ID NO.: 18 | GGSGGGGSG |
| H10F7-M11VL | SEQ ID NO.: 19 | DIQMTQSPSSLSASVGDRVT |
| | | ITCRASSGIISYIDWFQQKP |
| | | GKAPKRLIYATFDLASGVPS |
| | | RFSGSGSGTDYTLTISSLQP |
| | | EDFATYYCRQVGSYPETFGQ |
| | | GTKLEIKR |
| CL | SEQ ID NO.: 20 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |

*Note that the component CDRS of the VH and VL binders are in bold

ABBV-257 is a recombinant DVD-Ig comprised of 2 identical κ light chains and 2 identical IgG1 heavy chains covalently attached through a full complement of inter- and intra-molecular disulfide bonds. The disulfide linkage pattern is structurally similar to that of natural IgG1 antibodies. The human IgG1 constant region in ABBV-257 contains 2 mutations (L234A, L235A) in the lower hinge region that significantly reduce binding to Fcγ receptors, and 2 mutations (T250Q and M428L) that enhance its binding to neonatal Fc receptor (FcRn) at intracellular acidic pH to increase recycling and extend serum half-life of the molecule. The heavy chain is post-translationally modified by addition of N-linked glycans to the heavy chain at the same asparagine location commonly modified on IgG1 antibodies. The major glycans are fucosylated biantennary oligosaccharides containing 0, 1, or 2 galactose residues. Each light chain and heavy chain contains two variable domains connected in tandem by flexible glycine-serine peptide linker regions enabling dual specificity capable of binding both IL-17 and TNF in a tetravalent manner ABBV-257 has a molecular weight of 202 kDa and solubility of about 50 mg/mL at a minimum in formulation buffer. It is a lyophilisate powder at 50 mg/mL after reconstitution in histidine, sucrose, polysorbate-80. The drug product (ABBV-257 powder for solution for injection, 50 mg/mL, in vials) was stored refrigerated at 2° to 8° C. and protected from light. ABBV-257 selectively neutralizes human TNF and IL-17A and does not recognize a panel of other cytokines in the TNF or IL-17 families

### Example 3

### In Vitro Pharmacology of ABBV-257

The kinetic binding of ABBV-257 to TNF and IL-17 was determined using Biacore® surface plasmon resonance technology. The apparent association rate (ka) and dissociation rate (kd) were derived and used to calculate the overall equilibrium dissociation constant (KD) for the interaction. The results from several experiments indicated that ABBV-257 has very high affinity for both TNF and IL-17 as shown in Table 7.

TABLE 7
Binding Affinity of ABBV-257 for Recombinant TNF and IL-17

| Measure- | TNF | IL-17 A/A | IL-17A/F | |
|---|---|---|---|---|
| ment | Mean ± SD | Mean ± SD | Mean ± SD | N |
| ka | 3.3 ± 1.2 × 10⁶ | 1.1 ± 0.12 × 10⁶ | 3.4 ± 0.2 × 10⁵ | 3 |
| (M⁻¹s⁻¹) | | | | |
| kd (s⁻¹) | 1.6 ± 0.7 × 10⁵ | 3.0 ± 1.8 × 10⁻⁶ | 4.8 ± 0.4 × 10⁻⁶ | 3 |
| K_{D} (pM) | 4.9 ± 0.5 | 3.0 ± 2.1 | 14 ± 2 | 3 |

N = number of experiments

### Example 4

### In-Vitro Potency for ABBV-257 for Human TNF and IL-17

ABBV-257 fully neutralized human TNF and IL-17 bioactivity. The in vitro neutralization potency (inhibitory concentration 50%; IC50) of ABBV-257 was determined by measuring the amount of ABBV-257 required to inhibit 50% of either the TNF-induced lethality of L929 cells or the IL-17 dependent induction of IL-6 in fibroblasts. ABBV-257 neutralized both the A/A and A/F isoforms of IL-17, as shown in Table 8.

TABLE 8
In-Vitro Potency of ABBV-257 for Human TNF and IL-17

| | Mean IC50 ± SD (pM) | No. of Experiments |
|---|---|---|
| TNF | 25 ± 8 | 3 |
| IL-17 A/A | 26 ± 6 | 3 |
| IL-17 A/F | 110 ± 10 | 3 |

### Example 5

### Specificity of ABBV-257

The specificity of ABBV-257 for TNF and IL-17A was determined by assessing its binding to cytokines in the IL-17 and TNF families by direct enzyme-linked immunosorbent assay (ELISA). ABBV-257 bound to IL-17A and IL-17A/F heterodimer as expected but did not bind to IL-17B, IL-17C, IL-17D, or IL-17E (IL-25). Similarly, ABBV-257 bound to TNF, but not to the family members lymphotoxin a, 4-1BB ligand, LIGHT, APRIL, BAFF, OX40 ligand, CD30 ligand, TL1A, CD40 ligand, EDA-A2, RANK ligand, Fas ligand, TWEAK, and GITR ligand.

### Example 6

### In Vitro Species Cross Reactivity of ABBV-257

The cross reactivity of ABBV-257 to recombinant TNF (rTNF) and IL-17 of other species was assessed by determining the IC50 in an in vitro neutralization assay, as well as by determining the K_{D} using Biacore®, analysis. ABBV-257 binding protein neutralized monkey TNF and IL-17 with similar IC₅₀ compared to human (Table 9). In contrast, the IC₅₀ for rodent and rabbit IL-17 was markedly increased compared to human and did not neutralize rodent or rabbit TNF. Consistent with these findings, the KD of ABBV-257 for monkey TNF and IL-17 was similar to those in humans, and was increased for rodent and rabbit IL-17, correlating with the increased IC₅₀ in the bioassay (Table 10). No binding to rodent or rabbit TNF was detected even at very higher concentrations of rTNF. The full neutralization of monkey rTNF and rIL-17 in the bioassays supported the selection of cynomolgus monkey as a pharmacologically appropriate species for toxicological testing of ABBV-257. In addition, the lack of neutralization of rodent and rabbit TNF precluded the use of these species for toxicological studies.

TABLE 9
In-Vitro Potency of ABBV-257 for
Monkey and Rodent TNF and IL-17

| Species | IL-17 | TNF | N |
|---|---|---|---|
| Human | 26 ± 6 | 25 ± 8 | 3 |
| Monkey (Rhesus/Cynomolgus) | 32 ± 4 | 25 ± 7 | 3 |
| Mouse | 239 ± 67 | NI | 3 |
| Rat | 135 ± 24 | NI | 3 |
| Rabbit | 11400 ± 300 | NI | 3 |

N = number of experiments;
NI = not inhibited (at concentrations up to 1 μM)

TABLE 10
Binding Affinity of ABBV-257 for
Monkey and Rodent TNF and IL-17

| Species | IL-17 | TNF | N |
|---|---|---|---|
| Human | 3.0 ± 2.1 | 4.9 ± 0.5 | 3 |
| Monkey (Rhesus/Cynomolgus) | 11 ± 4.1 | 24 ± 4 | 3 |
| Mouse | 72 ± 2.9 | NB | 3 |
| Rat | <35 | NB | 3 |
| Rabbit | 3300 ± 100 | NB | 3 |

N = number of experiments;
NB = No binding (at concentrations up to 500 nM of rTNF).

### Example 7

### Functional Properties of Fc Domain of ABBV-257 and Activation of Immune Cells

In vitro assays were performed to characterize the Fc-effector function and the potential of ABBV-257 to activate immune cells. The Fc isotype of ABBV-257 is a human IgG1. The Fc region has been inactivated with regards to FcγR binding utilizing mutation of amino acids L240A L241A that reduce binding to Fcg receptors and Clq (Hezareh et al. (2002) J. Virol. 75(24):12161-12168; and Wine et al. (2000) J. Immunol. 164(10):5313-5318). As shown in Table 11, ABBV-257 significantly reduced binding to FcγR1, IIa (both 131H and R variants), IIb, and IIIa (158 H and V variants), which predicts a decreased ability to activate immune cells through antibody-dependent cell-mediated cytotoxicity.

ABBV-257 binding protein also demonstrated a decreased ability to bind complement component Clq. ABBV-257 contains 2 mutations in the constant regions CH2 (amino acid glutamine; abbreviated as Q) and CH3 (leucine, abbreviated as L) that increase its binding to FcRn at the lower pH found in the endosomal compartment. These mutations extend the serum half-life of ABBV-257.

TABLE 11
Fc Binding Characteristics of ABBV-257

| Function | Variant | | R |
|---|---|---|---|
| Fcγ RI binding | NA | Biacore | No significant binding |
| Fcγ RIIa | 131H | Biacore | No significant binding |
| Fcγ RIIa | 131R | Biacore | No significant binding |
| Fcγ RIIb | NA | Biacore | No significant binding |
| Fcγ RIIIa | 158F | Biacore | No significant binding |
| Fcγ RIIIa | 158V | Biacore | Lower than IgG1 control |
| | | | antibody |
| FcR_{N} | NA | Biacore | Increased binding |
| C1q binding | NA | ELISA | Lower than IgG1 control |
| | | | antibody |

NA = not applicable

### Example 8

### Human Peripheral Blood Cell Assay of ABBV-257

The ability of ABBV-257 DVD-Ig binding protein to bind or activate cellular components of human blood was assessed in vitro utilizing peripheral blood from healthy donors. The interaction of ABBV-257 with human peripheral blood was analyzed by flow cytometry from three human blood donors utilizing fluorescently tagged ABBV-257 (fluorescein isothiocyanate [FITC]; ABBV-257-FITC) binding protein. These data demonstrated minimal binding of ABBV-257-FITC to human peripheral blood cells. ABBV-257 did not cause any platelet aggregation following incubation at 100 μg/mL. ABBV-257 did not induce production of cytokines from peripheral blood cells in an ex vivo cytokine release assay in which whole blood from three human blood donors was incubated with plate-bound compound for 48 hours at 37° C. There was no statistically significant secretion of IL-1β, IL-1ra, IL-6, IL-8 (CXCL8), or TNF-α compared to a negative control antibody.

### Example 9

### ABBV-257 Pharmacokinetic Parameters after a Single Dose

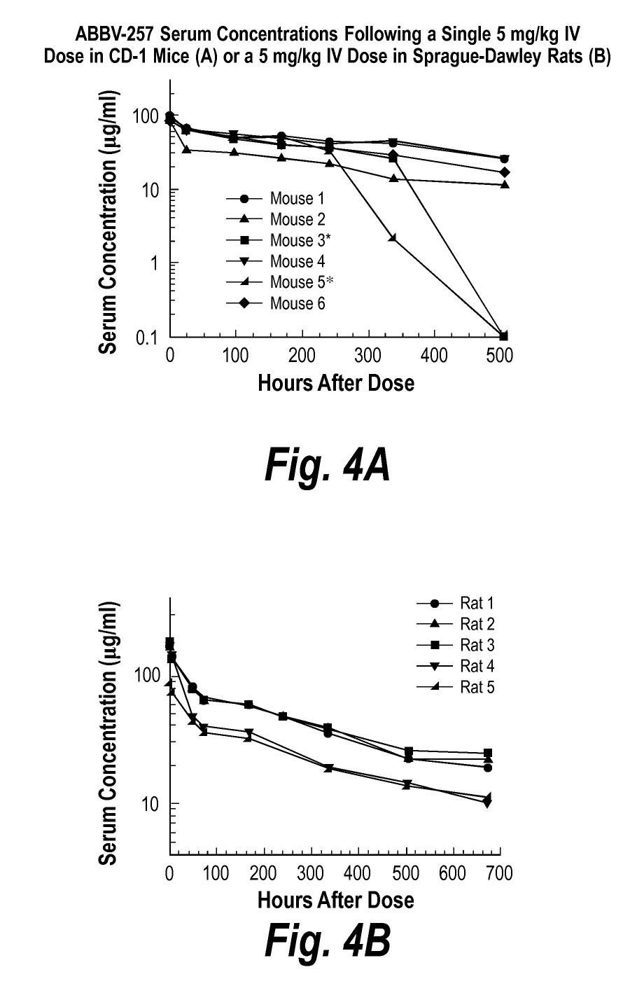

The pharmacokinetic profile of ABBV-257 following single IV doses in mouse and rat was characterized by low clearance values (0.2 and 0.15 mL/hr·kg in mice and rats, respectively), with low volumes of distribution (Vss=83.3 and 79.0 mL/kg in mice and rats, respectively). The terminal half-life in mice and rats was 12.9 and 17.5 days, respectively (Table 12). Serum exposure was maintained in 4/6 mice (FIG. 4, panel A) and in 5/5 rats (FIG. 4, panel B).

TABLE 12
ABBV-257 Pharmacokinetics Following a Single Intravenous Dose in CD-1 Mice
and Sprague-Dawley Rat

| | | | Mean (SD) | | | | |
|---|---|---|---|---|---|---|---|
| | Dose | t½ | V_{SS} | AUC_{0-inf} | CL | MRT | |
| Species | (mg/kg) | (day) | (mL/kg) | (mg · hr/mL) | (mL/hr · kg) | (days) | n |
| Mouse | 5 | 12.9 | 83.3 (24.9) | 29.1 (10.7) | 0.20 (0.09) | 18.8 (24.9) | 4 |
| Rat | 5 | 17.5 | 79.0 (23.4) | 36.8 (11.8) | 0.15 (0.05) | 22.7 (4.3) | 5 |

t½ = terminal half-life;
Vss = volume of distribution at steady state;
AUC0-inf = area under the concentration-time curve from time zero up to infinite time;
CL = clearance;
MRT = mean residence time

In cynomolgus monkey, ABBV-257 DVD-Ig binding protein serum exposures were not maintained throughout the study (up to 35 days) after a single 20 mg/kg dose of ABBV-257. The loss of exposure observed after Day 14 may have been due to the development of ADA.

### Example 10

### Human Peripheral Blood Cell Assay of ABBV-257

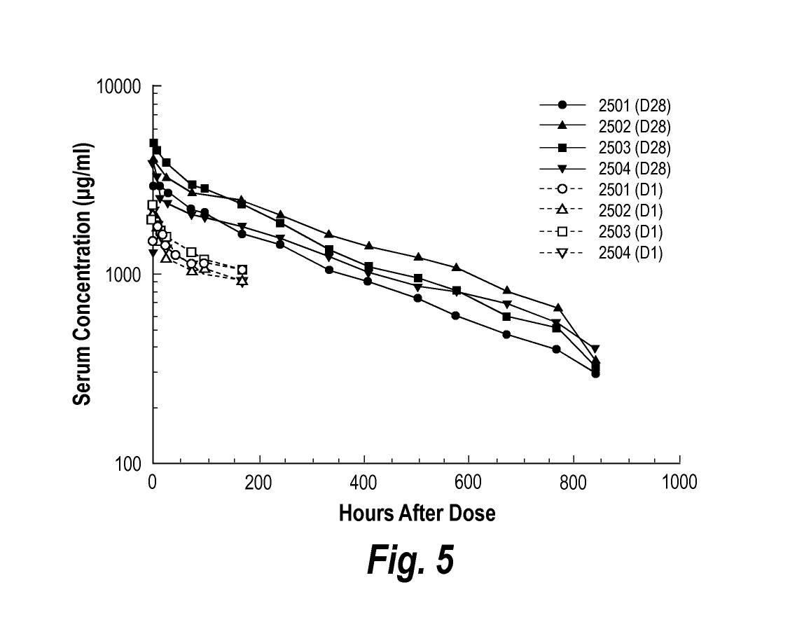

ABBV-257 DVD-Ig binding protein was administered weekly (4 doses total) via IV infusion to female cynomolgus monkeys at a dose (100 mg/kg), followed by a 5-week washout period (n=4 per group). The terminal half-life observed after the fourth dose was 13.0 days (FIG. 5).

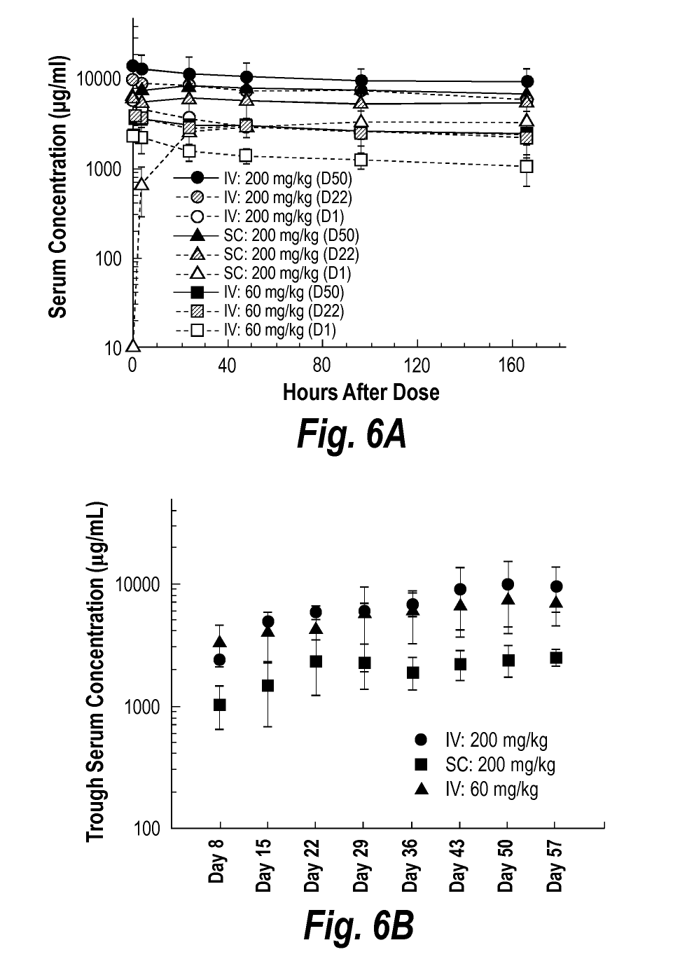

In the multiple dose Good Laboratory Practice (GLP) toxicity study, 2 groups of cynomolgus monkeys received 60 and 200 mg/kg doses of ABBV-257, administered as an intravenous bolus injection once per week for eight consecutive weeks. A third treatment group received a 200 mg/kg SC dose of ABBV-257 once weekly for eight consecutive weeks. Each treatment group contained four female and four male animals. The AUC and maximum concentration (Cmax) values increased in a dose-related fashion (FIG. 6; Table 13). Serum concentrations and AUC values for ABBV-257 did not appear to exhibit any sex-specific differences. The average of all AUC values in the 200 mg/kg SC dose group reached approximately 83% of the AUC values in the corresponding IV dose group. Peak plasma concentrations were noted 78 hours after the SC dose (average of Days 1, 22, and 50). Accumulation of ABBV-257 throughout the different dose groups was approximately a factor of 3, as indicated by an increase of the trough concentration (Ctrough) levels between Day 8 and Day 57.

TABLE 13
ABBV-257 Toxicokinetic Parameters After Intravenous
and Subcutaneous Injection in Cynomolgus Monkey
Following 8 Weeks of Once Weekly Dosing

| | ABBV-257 Dose (mg/kg) | | |
|---|---|---|---|
| Toxicokinetic | 60 IV | 200 IV | 200 SC |

| | |
|---|---|
| Parameter | Mean (SD) |

Day 1

| | | | |
|---|---|---|---|
| Number animals/ | 8 | 8 | 8 |
| group | | | |
| Cₘₐₓ (mg/mL) | 2.49 (0.668) | 6.05 (2.44) | 3.68 (1.02) |
| Cₘₐₓ/D | 0.042 (0.011) | 0.030 (0.012) | 0.0184 (0.005) |
| (mg/mL/mg/kg) | | | |
| Tₘₐₓ (hr) | N/A | N/A | 116.3 (44.3) |
| AUC | 230 (50.8) | 498 (53.8) | 494 (146) |
| (mg · hr/mL) | | | |
| AUC/D | 3.84 (0.848) | 2.49 (0.27) | 2.47 (0.73) |
| (mg · hr/mL/mg/kg) | | | |

Day 22

| | | | |
|---|---|---|---|
| Number animals/ | 6^{a} | 7 | 8 |
| group | | | |
| Cₘₐₓ (mg/mL) | 4.01 (0.782) | 10.9 (1.68) | 7.43 (2.74) |
| Cₘₐₓ/D | 0.067 (0.013) | 0.055 (0.008) | 0.037 (0.014) |
| (mg/mL/mg/kg) | | | |
| Tₘₐₓ (hr) | N/A | N/A | 92.3 (65.0) |
| AUC | 461 (95.5) | 1270 (175) | 971 (586) |
| (mg · hr/mL) | | | |
| AUC/D | 7.68 (1.6) | 6.33 (0.875) | 4.85 (2.93) |
| (mg · hr/mL/mg/kg) | | | |

Day 50

| | | | |
|---|---|---|---|
| Number animals/ | 6^{b} | 7^{c} | 8 |
| group | | | |
| Cₘₐₓ (mg/mL) | 3.76 (0.395) | 14.6 (6.29) | 9.86 (3.68) |
| Cₘₐₓ/D | 0.063 (0.007) | 0.073 (0.032) | 0.049 (0.018) |
| (mg/mL/mg/kg) | | | |
| Tₘₐₓ (hr) | N/A | N/A | 24.5 (17.4) |
| AUC | 477 (62.7) | 1770 (741) | 1290 (434) |
| (mg · hr/mL) | | | |
| AUC/D | 7.95 (1.05) | 8.83 (3.7) | 6.46 (2.17) |
| (mg · hr/mL/mg/kg) | | | |

N/A = not applicable;
Cmax/D = dose-normalized maximum concentration;
AUC/D = dose-normalized area under the concentration-time curve;
Tmax = time to maximum concentration
Data from Study TC13-084.
Tmax is not reported for IV dosing.
^{a}Two animals (at Day 22: 2002, 2502) excluded because of confirmed ADA response.
^{b}One animal (at Day 50: 2002) excluded because of confirmed ADA response; Animal 2502 was euthanized on Day 36.
^{c}One animal (at Day 22 and Day 50: 3502) excluded because of confirmed ADA response.

Two monkeys in the low dose group (60 mg/kg) and 1 monkey each in the 200 mg/kg IV and SC dose groups exhibited anti-ABBV-257 antibodies which correlated to a drop in exposure to the test article for the IV dosed animals. For the SC dosed ADA positive animal, the effect on the serum concentration profile was less obvious. Test-item induced ADA formation was not observed in any of the other animals and exposure of the animals to the test article was generally maintained.

### Example 11

### Toxicology Analysis of ABBV-257

The safety profile of ABBV-257 was evaluated in a GLP-compliant 8-week (8 doses) cynomolgus monkey toxicology study. In addition, a GLP-compliant tissue cross reactivity study was conducted using human tissues. IV and SC injection site tolerability was assessed during the 8-week toxicology study. The local tolerances of the vehicle/placebo formulations (without ABBV-257) were also qualified in a GLP-compliant rabbit local tolerability study. Cynomolgus monkey was the only species utilized for toxicology studies due to insufficient cross reactivity of ABB-257 to both TNF-α and IL-17 from mouse, rat, and rabbit species.

No adverse test article-dependent toxicities related to on-target or off-target binding of test article were observed during the GLP-compliant repeat-dose toxicology study using dose levels of 60 and 200 mg/kg IV, and 200 mg/kg SC.

During the 8-week toxicology study, one 60 mg/kg animal died following Dose 6. A comprehensive evaluation of the cumulative data from clinical observations, toxicokinetics, and anti-drug antibody analyses, serum circulating immune complex data, complement activation data, histologic evaluation of tissues, and immunohistochemical evaluation of immune complex deposition in tissues indicate the death was the result of exacerbation of an immune complex-mediated hypersensitivity reaction. The mortality was not attributed to a pharmacologic or toxicologic effect of test article administration.

Based upon a lack of adverse test article-related findings, the No Adverse Effect Level (NOAEL) during the 8-week repeat-dose toxicology study was 200 mg/kg/week among animals with sustained exposures. A summary of pivotal toxicology studies conducted with ABBV-257 is presented in Table 14.

TABLE 14
List of Pivotal Toxicology Studies Conducted with ABBV-257

| Type of | Species and | Method of | Duration | Doses^{a} |
|---|---|---|---|---|
| Study | Strain | Administration | of Dosing | (mg/kg/day) |
| Repeated- | Cynomolgus | IV, SC | 8 weeks | 60 IV, 200IV, |
| Dose | monkey | | | 200 SC once |
| Toxicity | | | | per week |
| Tissue | Human | — | — | — |
| Cross- | | | | |
| Reactivity | | | | |

Note that both the repeated-dose toxicity study and the tissue cross-reactivity study were GLP compliant.
IV = intravenous;
SC = subcutaneous;
IA = intra-arterial;
PV = paravenous;
IM = intramuscular
^{a}The NOAEL is underlined for GLP-compliant repeat-dose toxicity studies.

### Single Dose Toxicity

No single-dose toxicity studies were conducted. Analysis showed that no post-dose reactions or other ABBV-257-related effects were observed following the first dose of ABBV-257 among the 8-week repeat-dose toxicity studies described in the following section.

### Repeated Dose Toxicity—8-Week Toxicology Study of ABBV-257 by Intravenous Bolus and Subcutaneous Injection in Cynomolgous Monkeys

An 8-week GLP-compliant toxicity study was conducted in male and female cynomolgus monkeys at dose levels of 0 (placebo/vehicle; IV and SC), 60 mg/kg, or 200 mg/kg once/week IV bolus injection (3 to 5 minutes) and 200 mg/kg once per week SC injection (8 total doses/regimen). A preceding 4-week (4 dose, once/week) non-GLP repeat dose toxicokinetic/tolerability study at a single dose level of 100 mg/kg once/week indicated that serum test article exposures could be maintained at this dose level for 4 weeks.

Study parameters during the 8-week GLP-compliant repeat dose toxicology study included clinical signs, injection site observations, body weights, food evaluation, ophthalmologic and electrocardiologic examinations, clinical pathology (hematology, coagulation, clinical chemistry, urinalysis), toxicokinetic and ADA analyses, ADA parameters, ADA isotyping, circulating serum immune complex (CIC) values, gross necropsy, organ weight, histopathology and immunohistochemistry evaluation of immune complex deposition in tissues.

No adverse test article-dependent toxicities related to on-target or off-target binding of test article were observed during the GLP-compliant repeat-dose toxicology study using dose levels of 60 and 200 mg/kg IV, and 200 mg/kg SC.

Serum test article concentrations and toxicokinetic parameters for ABBV-257 did not exhibit any gender specific differences. Toxicokinetic values increased in a dose level and dose route related fashion throughout the dosing period. The 200 mg/kg IV dose and route produced the highest exposures; correlating to a Day 50 C max of 14.6 mg/mL and an AUC0-166 of 1770 mg·hr/mL.

Two 60 mg/kg IV animals (inclusive of 1 early death described below) and one 200 mg/kg IV animal exhibited ADA which corresponded to concurrently decreased ABBV-257 serum concentrations. Due to the ADA-altered systemic exposures, these three animals were excluded from mean toxicokinetic parameter calculations. One 200 mg/kg SC animal exhibited ADA that did not appear to negatively impact systemic exposure for this animal; therefore, the exposure data from this animal were not excluded from mean toxicokinetic calculations. The ADA observed in these 4 animals was IgG (not IgA, M, or E) isotype, and formed circulating immune complexes (ABBV-257/ADA complexes) in serum.

An acute post-dose response was present in a single female at 60 mg/kg/week IV, which lead to early death on Day 36 (approximately 15 minutes following Dose 6). The early death of this animal is most consistent with an immune complex-mediated hypersensitivity reaction based upon multiple study endpoints. Clinical signs following Dose 6 included unresponsiveness, no corneal reflex, faint heartbeat, and agonal breathing. The animal had IgG ADA titers corresponding to markedly decreased ABBV-257 concentrations; formation of circulating ABBV-257/ADA immune complexes; and complement activation following test article administration. Postmortem histopathologic changes suggestive of immune hypersensitivity included the following in the lung: minimal neutrophilic margination and thrombi in alveolar vessels, fibrin in alveoli, and mild histiocytic infiltration. Assessment of tissue-resident immune complex deposition by immunohistochemical techniques revealed that increased human IgG (interpreted as ABBV-257 DVD-Ig binding protein), monkey IgG and/or IgM (interpreted as ADA)-containing granular deposits in phagocytic cells in one or more tissues which were consistent with an immune complex (ABBV-257/ADA) basis for the post-dosing reaction and associated pathology in this animal. The mortality was not attributed to a pharmacologic or toxicologic effect of test article administration.

In conclusion, excluding the one early death attributable to an immune-mediated hypersensitivity response, no ABBV-257 dependent adverse effects were observed during the study. The 200 mg/kg IV animals produced the highest exposures (AUC 1770 mg·hr/mL on Day 50), which is the NOAEL among animals with sustained exposures.

### Tissue Cross-Reactivity

GLP-compliant tissue cross-reactivity studies were conducted using fluorescein labeled ABBV-257 DVD-Ig binding protein (2 and 10 μg/mL) and cryo-preserved tissues from human. At least 3 donor samples were evaluated for each tissue type. The tissue panel included all of the tissues identified in relevant regulatory guidance.

There was no fluorescein labeled ABBV-257 staining of the test human tissue cryosections, consistent with the low-grade expression of its target human epitopes in normal human tissues. There was no unexpected cross-reactivity. All assay control samples performed appropriately.

### Local Irritation

Test article injection site tolerance was evaluated during the 8-week repeat-dose toxicology studies. No injection site intolerance was observed via the IV and SC routes. A dedicated rabbit local tolerance study using ABBV-257 drug substance/drug product was not conducted.

### Example 12

### Study M14-355—a Phase 1 First-in-Human (FIH) Single Ascending Dose Study (Study M14-355) in Healthy Human Subjects

Clinical trial study M14-355 was performed and involved a single ascending dose, double-blind, randomized study planned for up to 40 healthy adult subjects to assess the safety, tolerability, and PK of ABBV-257 DVD-Ig binding protein with a single dose IV infusion or a single dose SC injection. Secondary objectives were to measure the ADA levels following a single IV or SC dose. An exploratory objective was to determine any change in biomarker assessments at multiple time points following study drug administration. The doses administered were 0.3 mg/kg (Group 1), 1.0 mg/kg (Group 2), and 3.0 mg/kg (Group 3) given IV and 0.3 mg/kg (Group 4) and 3 mg/kg (Group 4a) given SC. Eighteen subjects received IV doses and 12 subjects received SC doses of ABBV-257. Ten subjects received placebo control (6 in the IV administration arm and 4 in the SC administration arm).

### Pharmacokinetics in the First-in-Human Study of ABBV-257, Study M14-355

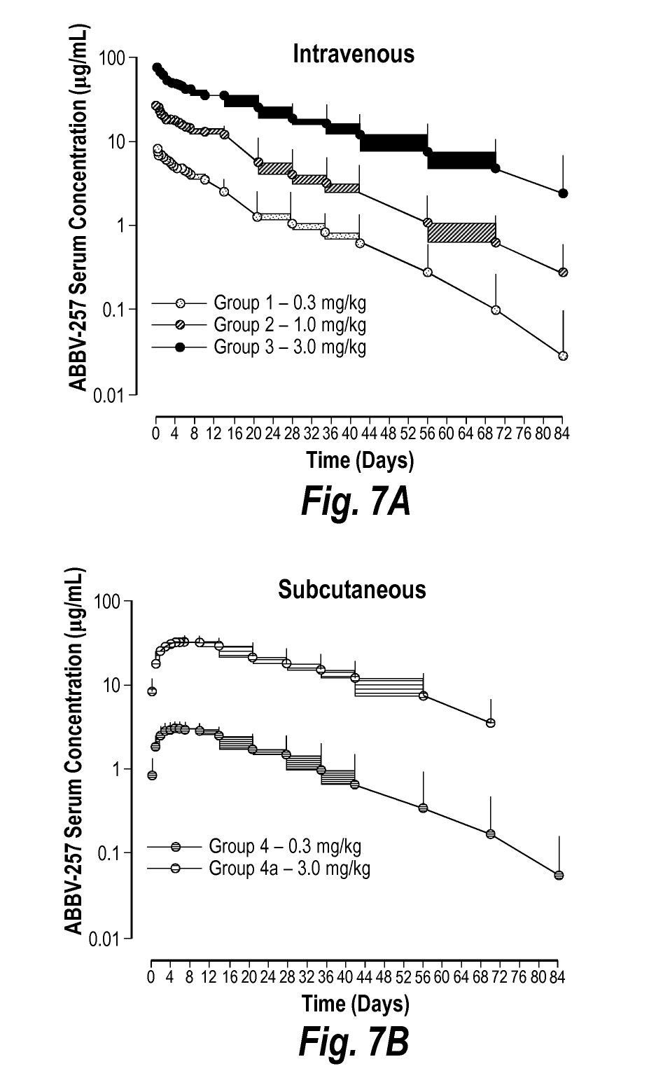

The mean and single-dose serum concentration-time profiles following an IV or SC dose of ABBV-257 are presented on a log-linear scale (FIG. 7 panel A and FIG. 7 panel B). See also Table 15.

The pharmacokinetics (Cmax and AUCinf) of ABBV-257 were slightly more than dose proportional following 0.3 to 3 mg/kg single dose range. The estimated bioavailability after SC administration was 74%.

TABLE 15
Mean (% CV) Pharmacokinetic Parameters Following a Single Dose of ABBV-257

| | Intravenous | | | | | | Subcutaneous | | | |
|---|---|---|---|---|---|---|---|---|---|---|
| | Group 1 | | Group 2 | | Group 3 | | Group 4 | | Group 5 | |
| Parameter | 0.3 mg/kg | | 1.0 mg/kg | | 3.0 mg/kg | | 0.3 mg/kg | | 3.0 mg/kg | |
| (Units) | N = 6 | | N = 6 | | N = 6 | | N = 6 | | N = 6 | |
| Cₘₐₓ (μg/mL) | 8.1 | (18) | 26.8 | (20) | 76.2 | (8) | 3.2 | (18) | 35.3 | (10) |
| Tₘₐₓ (hr)^{a} | 4.0 | (2-6) | 4.0 | (2-10) | 4.0 | (2-8) | 156 | (48-240) | 204 | (120-240) |
| Tmax (Day) | | | | | | | 6.5 | (2-10) | 8.5 | (5-10) |
| AUC_{0-Last} | 105 | (44) | 395 | (39) | 1460 | (35) | 90 | (53) | 982 | (38)^{b} |
| (μg · day/mL) | | | | | | | | | | |
| AUC_{0-inf} | 108 | (41) | 423 | (40)^{c} | 1545 | (43) | 91 | (53) | 1056 | (44)^{b} |
| (μg · day/mL) | | | | | | | | | | |
| CL (L/day)^{d} | 0.26 | (42) | 0.22 | (42)^{c} | 0.18 | (38) | 0.32 | (44) | 0.27 | (54)^{b} |
| t_{1/2} (Day)^{e} | 5.6 | (162) | 5.8 | (134) | 11.2 | (48) | 5.8 | (55) | 5.7 | (148)^{b} |
| Cₘₐₓ/Dose | 27.1 | (18) | 26.8 | (20) | 25.4 | (8) | 10.7 | (18) | 11.8 | (10) |
| (μg/mL)/(mg/kg) | | | | | | | | | | |
| AUCinf/Dose | 361 | (41) | 423 | (40)^{c} | 515 | (43) | 305 | (53) | 352 | (44)^{b} |
| (μg · day/mL)/(mg/kg) | | | | | | | | | | |

^{a}Median (range).
^{b}N = 4.
^{c}N = 5.
^{d}For SC dosing: apparent CL (CL/F: apparent total body clearance) is reported.
^{e}Harmonic mean (pseudo % CV); Terminal t½ may not be relevant because of the fast change in the slope at late time points.

The presence of ADA was measured with a validated immunoassay. Sampling for ADA occurred prior to ABBV-257 dosing (pre-dose) and following the single dose of ABBV-257 on Days 15, 22, 29, 36, 43, 57, 71 and 85. Complete preliminary ADA data are available for the first 4 dose groups and partial ADA data are available for the last dose group. ADA titers were detected in 23 out of 24 subjects in Groups 1 through 4. Of the 18 subjects who received ABBV-257 DVD-Ig binding protein in Groups 1 through 3, nine of the subjects had ADA associated with shorter half-life than the rest of the subjects, suggesting a negative impact of ADA on ABBV-257 exposure in these subjects. In the SC dose cohorts, 5 out of the 12 subjects who received ABBV-257 DVD-Ig binding protein (4 subjects in Group 4 and 1 in Group 4a) had ADA associated with shorter half-life compared to the rest of the subjects with lower ADA titer values in these dose groups. ADA detected in the study did not impact the safety or tolerability profile of ABBV-257 DVD-Ig binding protein.

### Safety and Efficacy

Efficacy was not assessed in Study M14-355. Eighteen subjects received IV doses and 12 subjects received SC doses of ABBV-257 DVD-Ig binding protein. Seven of the 18 subjects (7/18, 38.9%) who received ABBV-257 IV reported one or more AEs compared to the 4 of 6 subjects (4/6, 66.7%) who received placebo IV. Of the 12 subjects who received ABBV-257 SC, 3 (25.0%) reported at least one AE as compared to none of the 4 placebo recipients (Table 16).

Viral upper respiratory tract infection was the only preferred term reported for more than one subject (two subjects in the IV arm of the study, one placebo recipient and one subject who received a 0.3 mg/kg dose of ABBV-257 DVD-Ig binding protein). There were no deaths, SAEs, or AEs leading to discontinuation during the study. The only events considered possibly related to the study drug were injection site reaction in one subject in the ABBV-257 0.3 mg/kg SC group and hyperhidrosis in one subject in the ABBV-257 3.0 mg/kg IV group. Most AEs were mild in intensity; no severe AEs were reported. All AEs in the IV-dosed subjects were described as mild intensity and all were categorized as toxicity grade 1. One case of moderate post-traumatic pain and one case of mild rhabdomyolysis were reported in the 3.0 mg/kg SC group. This subject reported discomfort in muscles following weight lifting and alcohol consumption ten days following administration of study drug. Prior weight lifting and alcohol consumption of this subject were clinically asymptomatic and there were no concurrent laboratory abnormalities. Neither event was considered as having a reasonable possibility of being study drug related.

A female subject in Group 3 IV dosing of 3.0 mg/kg experienced an allergic reaction described as erythema and itching in her face and right hand starting 82 days post study drug administration, which was mild in intensity and was treated with steroids. The subject reported definite exposure to a pet that had been in contact with poison ivy the day before the onset of symptoms. The subject recovered after twelve days, and the allergic reaction was assessed as not related to study drug.

Overall there was no apparent dose relationship in frequency, type, or intensity of AEs in Study M14-355 following ABBV-257 DVD-Ig binding protein administration via IV or SC routes. All the infections reported in the study were mild in severity and not related to study drug. There were no systemic hypersensitivity reactions reported. Anti-drug antibodies detected in the study did not appear to impact the AE profile of ABBV-257 DVD-Ig binding protein.

TABLE 16
Number and Percentage of Subjects with Treatment-Emergent Adverse Events
by Primary MedDRA System Organ Class and Preferred Term

| | Intravenous | | | | Subcutaneous | | |
|---|---|---|---|---|---|---|---|
| System Organ | ABBV-257 mg/kg | | | | ABBV-257 mg/kg | | |
| Class | Placebo | 0.3 | 1.0 | 3.0 | Placebo | 0.3 | 3.0 |
| Preferred | N = 6 | N = 6 | N = 6 | N = 6 | N = 4 | N = 6 | N = 6 |
| Term^{a} | N (%) | n (%) | n (%) | n (%) | n (%) | n (%) | n (%) |
| Any adverse event | 4 (66.7) | 2 (33.3) | 2 (33.3) | 3 (50.0) | 0 | 1 (16.7) | 2 (33.3) |

Gastrointestinal disorders

| | | | | | | | |
|---|---|---|---|---|---|---|---|
| Abdominal pain | 0 | 0 | 1 (16.7) | 0 | 0 | 0 | 0 |
| Diarrhoea | 0 | 0 | 1 (16.7) | 0 | 0 | 0 | 0 |
| Vomiting | 0 | 1 (16.7) | 0 | 0 | 0 | 0 | 0 |

General disorders and administration site conditions

| | | | | | | | |
|---|---|---|---|---|---|---|---|
| Fatigue | 1 (16.7) | 0 | 0 | 0 | 0 | 0 | 0 |
| Infusion site haematoma | 0 | 0 | 1 (16.7) | 0 | 0 | 0 | 0 |
| Injection site reaction | 0 | 0 | 0 | 0 | 0 | 1 (16.7) | 0 |
| Local swelling | 1 (16.7) | 0 | 0 | 0 | 0 | 0 | 0 |

Immune system disorders

| | | | | | | | |
|---|---|---|---|---|---|---|---|
| Hypersensitivity | 0 | 0 | 0 | 1 (16.7) | 0 | 0 | 0 |

Infections and infestations

| | | | | | | | |
|---|---|---|---|---|---|---|---|
| Viral upper respiratory | 1 (16.7) | 1 (16.7) | 0 | 0 | 0 | 0 | 0 |
| tract infection | | | | | | | |

Injury, poisoning and procedural complications

| | | | | | | | |
|---|---|---|---|---|---|---|---|
| Eye penetration | 0 | 0 | 0 | 1 (16.7) | 0 | 0 | 0 |
| Ligament sprain | 0 | 0 | 0 | 1 (16.7) | 0 | 0 | 0 |
| Post-traumatic pain | 0 | 0 | 0 | 0 | 0 | 0 | 1 (16.7) |
| Tooth fracture | 1 (16.7) | 0 | 0 | 0 | 0 | 0 | 0 |

Musculoskeletal and connective tissue disorders

| | | | | | | | |
|---|---|---|---|---|---|---|---|
| Rhabdomyolisis | 0 | 0 | 0 | 0 | 0 | 0 | 1 (16.7) |

Nervous system disorders

| | | | | | | | |
|---|---|---|---|---|---|---|---|
| Presyncope | 1 (16.7) | 0 | 0 | 0 | 0 | 0 | 0 |

Respiratory, thoracic and mediastinal disorders

| | | | | | | | |
|---|---|---|---|---|---|---|---|
| Epistaxis | 1 (16.7) | 0 | 0 | 0 | 0 | 0 | 0 |
| Oropharyngeal pain | 1 (16.7) | 0 | 0 | 0 | 0 | 0 | 0 |

Skins and subcutaneous tissue disorders

| | | | | | | | |
|---|---|---|---|---|---|---|---|
| Hyperhidrosis | 0 | 0 | 0 | 1 (16.7) | 0 | 0 | 0 |

^{a}MedDRA version 17.0.

All treatment-emergent adverse events experienced by at least 1 subject receiving ABBV-257, regardless of causality, include gastrointestinal disorders (abdominal pain, diarrhea, vomiting), general disorders and administration site conditions (fatigue, infusion site, hematoma, injection site reaction, local swelling), immune system disorders (hypersensitivity), infections and infestations (viral upper respiratory tract infection), injury, poisoning and procedural complications (eye, penetration, ligament sprain, post-traumatic pain, tooth fracture), musculoskeletal and connective tissue disorders (rhabdomyolysis), nervous system disorders (presyncope), respiratory, thoracic and mediastinal disorders (epistaxis, oropharyngeal pain), and skin and subcutaneous tissue disorders (hyperhidrosis).

In general, the incidence of potentially clinically significant laboratory values was low. There were no AEs related to laboratory abnormalities. Grade 3 or 4 potentially clinically significant laboratory values were reported for neutrophils, creatine phosphokinase (CPK), alanine aminotransferase (ALT), aspartate aminotransferase (AST), triglycerides, and urine protein with the highest incidence of such values occurring for CPK (Table 18). With regard to the potentially clinically significant laboratory values, there was no obvious pattern or evident correlation with dose or route of administration. There were no apparent differences in the potentially clinically significant laboratory values between placebo and ABBV-257 DVD-Ig binding protein.

Of the IV-dosed subjects, two subjects (one placebo recipient and one subject in the 0.3 mg/kg dose group) had a potentially clinically significant decrease in heart rate (<50 bpm and ≧15 bpm decrease) and one subject in the 1.0 mg/kg dose group had a potentially clinically significant increase in heart rate (>120 bpm and ≧15 bpm increase). None of these observations was considered clinically significant and no AEs reported in the study were related to vital signs or ECG findings. There were no other subjects with a potentially clinically significant vital sign or ECG value.

TABLE 17
Number and Percentage of Subjects with Potentially Clinically Significant Grade
3 or 4 Laboratory Values - Study M14-355

| | IV | | | | SC | | |
|---|---|---|---|---|---|---|---|
| | ABBV-257 | | | | ABBV-257 | | |
| | Placebo | 0.3 mg/kg | 1.0 mg/kg | 3.0 mg/kg | | 0.3 mg/kg | 3.0 mg/kg |
| Variable | N = 6 | N = 6 | N = 6 | N = 6 | Placebo | N = 6 | N = 6 |
| Grade | n (%) | n (%) | n (%) | n (%) | N = 4 | n (%) | n (%) |
| Neutrophils | | | | | | | |
| Grade 3 | 0 | 1 (16.7) | 1 (16.7) | 0 | 0 | 0 | 0 |
| CPK | | | | | | | |
| Grade 3 | 1 (16.7) | 0 | 0 | 0 | 0 | 1 (16.7) | 0 |
| Grade 4 | 1 (16.7) | 1 (16.7) | 0 | 1 (16.7) | 1 (25.0) | 1 (16.7) | 1 (16.7) |
| AST | | | | | | | |
| Grade 3 | 1 (16.7) | 0 | 0 | 0 | 0 | 0 | 0 |
| Grade 4 | 0 | 0 | 0 | 0 | 0 | 0 | 1 (16.7) |
| ALT | | | | | | | |
| Grade 3 | 0 | 0 | 0 | 0 | 0 | 0 | 1 (16.7) |
| Triglycerides | | | | | | | |
| Grade 3 | 0 | 0 | 0 | 0 | 1 (25.0) | 0 | 0 |
| Urine protein | | | | | | | |
| Grade 3 | 0 | 0 | 0 | 0 | 0 | 0 | 1 (16.7) |

Toxicity grading scale for healthy adult and adolescent volunteers enrolled in preventative vaccine clinical trials (2007); the value must also be more extreme than the baseline value.

First-in-Human (FIH) Clinical Study M14-355

Examples herein discussed the FIH study of ABBV-257 (Study M14-355), which enrolled healthy adult volunteers to assess the safety, tolerability, pharmacokinetics, and ADA profile of a single dose of ABBV-257 DVD-Ig binding protein without the confounding effects of concomitant disease or therapy. Potential risks with this study were addressed by the protocol-defined inclusion and exclusion criteria, study design features, and monitoring procedures outlined herein, and specified in the protocol.

Part 1 of Study M14-355 was a randomized, double-blind, placebo-controlled design to assess the safety, tolerability, pharmacokinetics and immunogenicity (via ADA assessment) of a single IV infusion of ABBV-257 DVD-Ig binding protein. This part of the study was conducted in 24 subjects in 3 groups (Groups 1 to 3), with 8 subjects in each group. Within each group, 6 subjects were randomized to receive ABBV-257 and 2 subjects received matching placebo. The ABBV-257 dose administered in Group 1 was 0.3 mg/kg IV. The subsequent ABBV-257 doses were 1.0 mg/kg and 3.0 mg/kg IV for Groups 2 and 3, respectively. Part 2 of Study M14-355 was a randomized, double-blind, placebo-controlled design to assess the safety, tolerability, pharmacokinetics and immunogenicity (via ADA assessment) of a single SC injection of ABBV-257. This part of the study was conducted in 16 subjects in two groups (Groups 4 and 4a), with eight subjects in each group. Within each group, six subjects were randomized to receive ABBV-257 and 2 subjects received matching placebo. The ABBV-257 doses administered were 0.3 mg/kg SC and 3 mg/kg SC for Groups 4 and 4a, respectively.

Preliminary pharmacokinetic data indicated ABBV-257 DVD-Ig binding protein exposure to be slightly more than dose proportional following 0.3 to 3.0 mg/kg dose range. The preliminary bioavailability estimate after SC administration was ˜74%. The majority of subjects in the FIH study had detectable ADA, within 2 weeks of dosing. ADA detected in the study did not impact the safety or tolerability profile of ABBV-257 DVD-Ig binding protein.

Preliminary safety data indicate that ABBV-257 DVD-Ig binding protein has an acceptable safety and tolerability profile. There were no deaths, SAEs, or discontinuations due to AEs during Study M14-355. Most of the AEs reported have been mild in severity. All infections were mild in severity and not related to study treatment. No systemic hypersensitivity reactions were reported. The only AE experienced by more than one subject was viral upper respiratory tract infection, which occurred in two subjects in the IV group (one placebo recipient and one subject who received a 0.3 mg/kg dose of ABBV-257). In general, the incidence of potentially clinically significant abnormal laboratory values was low, with CPK being the most common potentially clinically significant abnormal laboratory value. Laboratory abnormalities seen following placebo or ABBV-257 DVD-Ig binding protein were comparable. No concerning patterns of AEs or laboratory findings were reported. Overall there was no apparent dose relationship in frequency, type, or intensity of AEs or laboratory abnormalities in Study M14-355 following IV or SC administration of ABBV-257 DVD-Ig binding protein. There were no observed clinically significant vital sign or ECG abnormalities. ADA detected in the study did not impact the safety or tolerability profile of ABBV-257 DVD-Ig binding protein. No dose limiting toxicities were observed during the study. Safety The potential safety concerns for administration of ABBV-257 DVD-Ig binding protein were the risk of systemic hypersensitivity reactions and an increased risk of infection. There was no evidence for either of these safety concerns in Study M14-355. Additionally, with over 100 subjects currently exposed to single or multiple doses of another DVD-Ig targeting TNF and IL-17) no systemic hypersensitivity reactions have been reported. One subject in the 3.0 mg/kg IV group experienced a mild localized rash, redness, itching on the right side of her face and right hand with onset 82 days after she received ABBV-257 3.0 mg/kg IV. The subject recovered after 12 days, and the event reported as an allergic reaction was assessed as not related to study drug by the investigator. Infections reported during Study M14-355 were mild viral upper respiratory tract infections assessed as not related to study treatment, which occurred in two subjects (one placebo recipient and one subject in the 0.3 mg/kg dose group).

The risk of other AEs that have been associated with the anti-TNF agents, including malignancy, central nervous system demyelinating disease, pancytopenia (including aplastic anemia), worsening or new onset heart failure, and lupus-like syndrome, was low given the limited duration of exposure in this study in healthy volunteers, the application of protocol-specified exclusion criteria and safety monitoring procedures in the study protocol. No such events were reported in Study M14-355.

However, several precautions were taken in the planned multiple ascending dose Study M14-439 (Example 13) to mitigate the risk of potential systemic hypersensitivity reactions with ABBV-257 DVD-Ig binding protein. To address the risk for infection or hypersensitivity reactions in humans who receive ABBV-257, the study protocol implemented enrollment criteria, screening procedures, and a clinical schedule and monitoring plan to mitigate, monitor, and manage potential hypersensitivity reactions, other systemic reactions, and infections.

### Example 13

### Clinical Study Protocol M14-439—a Randomized, Double-Blind, Placebo-Controlled Study in Subjects with Rheumatoid Arthritis to Evaluate the Safety, Tolerability and Pharmacokinetics of Multiple Doses of ABBV-257

This Phase 1, randomized, double-blind, placebo-controlled, multiple-dose study was designed to assess the safety, tolerability, pharmacokinetics and immunogenicity of different dose levels of ABBV-257 given with methotrexate (MTX). Adult male and female subjects with RA were selected to participate in the study according to the selection criteria.

The study was designed to enroll 24 subjects to meet scientific and regulatory objectives without enrolling an undue number of subjects in alignment with ethical considerations.

After meeting the selection criteria, enrolled subjects were randomly assigned in 3:1 ratio to either ABBV-257 or placebo, in sequential dose groups as shown in Table 18.

TABLE 18
Planned Dose Groups

| | | Number of Subjects |
|---|---|---|
| Group^{a} | Regimen^{b,c} | Active:Placebo |
| 1 | 30 mg of ABBV-257 or placebo | 6:2 |
| | SC EOW dosing (4 doses) | |
| 2 | 100 mg of ABBV-257 or placebo | 6:2 |
| | SC EOW dosing (4 doses) | |
| 3 | 300 mg of ABBV-257 or placebo | 6:2 |
| | SC EOW dosing (4 doses) | |

^{a}Subjects may not participate in more than one dosing group
^{b}Dose level or dosing frequency may be adjusted based on the available safety, tolerability, and PK data from previous dose group(s).
^{c}Subjects receive their stable MTX dose weekly.

Study drug (ABBV-257 or placebo) was administered on Study Days 1, 15, 29, and 43 for the EOW dosing. The first three subjects of the first dose group were dosed at least 24 hours apart. The remaining subjects within a dose group were dosed up to 2 subjects per day. Subjects continued their weekly MTX dosing throughout participation in the study.

Dosing for Groups 2 and 3 were sequentially enabled upon the review of safety data through administration of the study drug at approximately Day 15 of the last subject in the precedent dose group. The subsequent dosing scheme was adjusted (e.g., dosing interval, number of doses) based on PK and safety data from previous group(s). Subjects were confined to the study site and supervised for periods of approximately 72 hours for the first and last doses of study drug. Confinement for the first dose began on Study Day—1. Subjects remained at the study site and were supervised for at least 2 hours following the second and third doses of study drug. Confinement for the last dose began on Day 42. Each confinement period ended after completion of all study procedures on the scheduled day of discharge.

Subjects had outpatient visits between confinement periods as indicated in Table 19. Safety was assessed throughout the study. Pharmacodynamic effects of ABBV-257 were investigated by exploratory disease response measures, biomarkers and fluorescence optical imaging (FOI) in patients with tender/swollen joints in hands as indicated in Table 19. From subjects who consent, a blood sample is collected to obtain a sample of genetic material (DNA/RNA). These DNA/RNA samples were used to study genetic factors contributing to the subject's response to ABBV-257 in terms of pharmacokinetics, pharmacodynamics, and safety.

### Selection of Study Population

Subjects underwent screening procedures within 30 days prior to initial study drug administration. Adult male and female subjects in general good health who met the inclusion criteria and who did not meet any of the exclusion criteria were eligible for enrollment into the study. Subjects that initially screen-failed for the study were permitted to re-screen one time following a repeat of all screening procedures with the possible exceptions noted below. The subject had to meet all inclusion and none of the exclusion criteria at the time of re-screening in order to qualify for the study. There was no minimum period of time a subject had to wait to re-screen for the study. If the subject had a complete initial screening visit including the assessment of a PPD test (or equivalent) and chest x-ray (CXR), these two tests were not required to be repeated for the re-screening visit. Adult male and female subjects with RA were selected to participate in the study according to the selection criteria.

### Inclusion Criteria

A subject was eligible for study participation if he/she met the following criteria:
1. Male or female and age is between 18 and 75 years, inclusive.
2. Diagnosis of RA based on either the 1987 revised ACR classification criteria or the 2010 American College of Rheumatology (ACR)/European League against Rheumatism (EULAR) criteria ≧3 months.
3. Except for MTX, the subject must have discontinued all disease modifying anti-rheumatic drugs (DMARD) for at least 5 half-lives before the first dose of study drug, and undergone cholestyramine washout if received Leflunomide within the past 3 months.
4. Subject must have been on methotrexate therapy ≧3 months and on a stable dose (7.5-25 mg/week) for at least 4 weeks prior to the first dose of study drug. Subject must be able to continue on stable dose of MTX for the duration of study participation.
5. If female, subject must meet one of the following criteria:
   Postmenopausal (defined as no menses for at least 1 year, with no alternate cause for amenorrhea)
   Surgically sterile (bilateral oophorectomy or hysterectomy)
   Women not in one of the above two categories were considered of child bearing potential and must use one of the following methods of birth control:
      combined (estrogen and progestogen containing) hormonal contraception associated with inhibition of ovulation started at least 2 months prior to randomization: oral, intravaginal, or transdermal
         progestogen-only hormonal contraception associated with inhibition of ovulation started at least 2 months prior to randomization: oral, injectable, or implantable
         intrauterine device (IUD)
         intrauterine hormone-releasing system (IUS)
         bilateral tubal occlusion/ligation
         Vasectomized partner (procedure at least 6 months earlier, the vasectomized male partner should be the sole partner for that female subject)
         sexual abstinence (refraining from heterosexual intercourse during the entire study period)
6. Females must have negative results for pregnancy tests performed:
   at Screening on a urine specimen obtained within 30 days prior to initial study drug administration, and
   prior to dosing on a serum sample obtained on Study Day—1.
7. If male, subject must agree not to donate sperm starting on the first day of confinement until 5 months after last dose of study drug.
8. If male, subject (including those who have had vasectomies) should use condoms from the first dose of study drug until 5 months after the last dose of study drug.
9. Body Mass Index (BMI) is 19 to 35, inclusive. (BMI is calculated as weight [kg] divided by height [m²].)
10. Judged to be in good general health as determined by the Investigator based upon the results of medical history, laboratory profile, physical examination and 12-lead electrocardiogram (ECG) performed at Screening.
11. Must voluntarily sign and date each informed consent, approved by an Independent Ethics Committee (IEC)/Institutional Review Board (IRB), prior to the initiation of any screening or study-specific procedures.

### Exclusion Criteria

A subject was not eligible for study participation if he/she met any of the following criteria:
1. Female who is pregnant or breastfeeding.
2. Female subject who is considering becoming pregnant during the study or for approximately 5 months after the last dose of study drug or male subject who is considering fathering a child during the study or for approximately 5 months after the last dose of study drug.
3. History of clinically significant drug or alcohol abuse in the 6 months prior to initial study drug administration.
4. Positive screen for drugs of abuse or alcohol at Screening or upon initial confinement.
5. Evidence of anti-ABBV-257 antibody results in a pre-study serum sample.
6. History of significant allergic reaction or significant sensitivity to any constituents of the study drug formulation; or history of anaphylactic reaction to any agent (e.g., food products and bee sting); or history of a major reaction to any IgG-containing product; or known or suspected allergy to FOI fluorescent agent or iodine.
7. Evidence of dysplasia or history of malignancy (including lymphoma and leukaemia) other than a successfully treated non-metastatic cutaneous squamous cell or basal cell carcinoma or localized carcinoma in situ of the cervix.
8. History of persistent chronic or active infection(s) requiring hospitalization or treatment with intravenous or oral antimicrobials/antibiotics within 30 days prior to initial study drug administration.
9. HBs Ag positive (++) or detected sensitivity on the HBV-DNA PCR qualitative test for HBc Ab/HBs Ab positive subjects; or history or evidence of active hepatitis C infection.
10. History of or positive Screening test for human immunodeficiency virus (HIV Ab) infection; or a history of any genetic, congenital, or acquired immunodeficiency syndrome. Negative HIV status is confirmed at Screening and the results were maintained confidentially by the study administration.
11. History or evidence of active tuberculosis (TB). Subjects were evaluated for latent TB infection. Subject must demonstrate absence of TB infection or exposure by a negative QuantiFERON-TB Gold at Screening.
12. In the opinion of the investigator, the subject has evidence of risk factors for latent TB.
13. Has received any investigational drug product of chemical or biologic nature within 30 days or 5 half-lives of the drug (whichever is longer) prior to initial study drug administration.
14. Has a history of any clinically significant respiratory, renal, hepatic, gastrointestinal, hematologic disorder, non-healing wounds or recurrent poor wound healing, or any uncontrolled medical illness, neurologic symptoms of demyelinating disease.
15. Recent history of a psychiatric illness that in the opinion of the Investigator could interfere with compliance to the protocol.
16. Known medical diagnosis of persistent asthma, chronic obstructive pulmonary disease if it could impact participation in the study; or significant atopy requiring daily therapy; history or diagnosis of mastocytosis or clonal mast cell disorder.
17. Febrile illness within 1 week prior to dosing.
18. History of chronic recurrent or persistent infections (including mucocutaneous candidiasis).
19. Has undergone major surgery within the 2 months prior to the initial study drug administration.
20. Donation or loss of 550 mL or more blood volume (including by plasmapheresis) or receipt of a transfusion of any blood product within 8 weeks prior to initial study drug administration.
21. Clinically significant abnormal ECG including ECG with QTcF >450 msec, PR interval >220 msec, or other clinically significant baseline abnormalities as judged by the Investigator at Screening or Study Day—1.
22. Myocardial infarction, coronary stenting, or CVA within the 1 year prior to initial study drug administration or greater than Class 1 angina pectoris or clinically significant aortic stenosis.
23. Cardiac failure at time of Screening >NYHA Class 2.
24. Confirmed systolic blood pressure measurement >160 mmHg systolic and >100 mmHg diastolic on Study Day—1.
25. History of diabetes mellitus (DM), HbAlc of ≧6.5% at Screening or fasting plasma glucose (FPG) ≧126 mg/dL (7.0 mmol/L) at Screening.
26. Confirmed hemoglobin ≦9 gm/dL or platelet count <100,000 μ/L or WBC <3000 μ/L or absolute neutrophil count <1500 μ/L at Screening.
27. Clinically significant abnormal screening laboratory results as evaluated by the Investigator, including serum values of AST or ALT greater than 2.25× the upper limit of normal, or creatinine greater than 1.5× the upper limit of normal, or absolute neutrophil count <1500 μ/L.
28. Subject has received vaccination with a live viral agent (including live attenuated influenza vaccine via nasal spray)≦to 1 month prior to Screening or requires vaccination during study participation and up to approximately 5 months (at least 5× the estimated half-life for ABBV-257) after the last dose of study drug.
29. Subject is unable to washout prohibited medications.
30. Concurrent use of other immunosuppressant medications other than those allowed as specified in the protocol.
31. Subject has any medical condition or illness other than RA that is not well controlled with treatment that would, in the opinion of the Investigator, preclude study participation or interfere with other symptoms of RA.
32. Subject who has been legally institutionalized.
33. Current enrollment in another investigational study.
34. Consideration by the Investigator, for any reason that the subject is an unsuitable candidate to receive ABBV-257.

### Prior and Concomitant Therapy

Subject must have been on methotrexate therapy >3 months and on a stable dose (7.5-25 mg/week) for at least 4 weeks prior to the first dose of study drug. Subjects continued taking MTX as prescribed in addition to receiving study drug (ABBV-257 or placebo) throughout the duration of the study. Reduction in the dose of MTX was not allowed. If the subject could not tolerate their dose of MTX, he/she was discontinued from the study.

If a subject reported taking any over-the-counter or prescription medications, vitamins and/or herbal supplements or if administration of any medication became necessary from 2 weeks prior to study drug administration through the end of the study, the name of the medication, dosage information including dose, route and frequency, date(s) of administration including start and end dates, and reason for use was recorded, and the study designated physician notified.

In some circumstances, the subject may be asked to use a methotrexate acid dosing diary. The diary might have the subject note the amount, time, and dose of methotrexate taken, along with an area for any additional comments he or she that might want to record.

### Efficacy, Pharmacokinetic, Pharmacodynamic, Pharmacogenetic and Safety Assessments/Variables

### Efficacy and Safety Measurements Assessed and Flow Chart

This study was not designed to assess efficacy, however, disease response, biomarker and FOI data were collected for exploratory analysis as pharmacodynamic variables.

TABLE 19
Study Activities

| | SCR^{a} | | | | | | | | | | | | |
|---|---|---|---|---|---|---|---|---|---|---|---|---|---|
| Procedure | (D-30 to D-2) | D-1 | D1 | D2 | D3 | D4 | D5 | D6 | D8 | D11 | D15 | D22 | D29 |
| Informed Consent^{b} | X | | | | | | | | | | | | |
| Confinement^{c} | | X | X | X | X | | | | | | | | |
| Outpatient Visit | X | | | | | | X | | X | | X | X | X |
| ABBV-257 | | | X | | | | | | | | X^{d} | | X^{d} |
| Administration | | | | | | | | | | | | | |
| Medical/Surgical | X | X | | | | | | | | | | | |
| Latent TB Risk Factor | X | | | | | | | | | | | | |
| Questionnaire | | | | | | | | | | | | | |
| Physical Examination^{f} | X | X | | | | | | | | | | | |
| Body Weight | X | | X | | | | | | | | | | |
| Vital Signs | X | X | X^{g,} | X | X | | X | | X | | X | X | X |
| Urine Pregnancy Test^{l} | | X | | | | | | | | | | | |
| Serum Pregnancy Test | X | | | | | | | | | | | | |
| 12-Lead ECG | X^{j} | X^{k,l} | X^{g,k,l} | | X^{k,l} | | X^{k,l} | | X^{k,l} | | | | |
| HbA1c | X | | | | | | | | | | | | |
| PT, PTT, INR | X | | | | | | | | | | | | |

| | | | | | | | | | | | | | | PD |
|---|---|---|---|---|---|---|---|---|---|---|---|---|---|---|
| Procedure | D36 | D42 | D43 | D44 | D45 | D47 | D50 | D57 | D71 | D85 | D103 | D133 | D193 | Visit |
| Informed Consent^{b} | | | | | | | | | | | | | | |
| Confinement^{c} | | X | X | X | X | | | | | | | | | |
| Outpatient Visit | X | | | | | X | X^{m} | X | X | | X | X | X | X |
| ABBV-257 | | | X | | | | | | | | | | | |
| Administration | | | | | | | | | | | | | | |
| Medical/Surgical | | | | | | | | | | | | | | |
| Latent TB Risk Factor | | | | | | | | | | | | | | |
| Questionnaire | | | | | | | | | | | | | | |
| Physical Examination^{f} | | | | | | | | | | | X | X | X | X |
| Body Weight | | | | | | | | | | | X | X | | X |
| Vital Signs | X | X | X^{l} | X | X | X | X | X | X | | X | X | X | X |
| Urine Pregnancy Test^{l} | | | | | | | | | | | | | | |
| Serum Pregnancy Test | | X | | | | | | | | | X | X | | X |
| 12-Lead ECG | | | X^{k,l} | | X^{k,l} | X^{k,l} | X^{k,l} | | | | X^{j} | X^{l} | X^{l} | X^{j} |
| HbA1c | | | | | | | | | | | | | | |
| PT, PTT, INR | | | | | | | | | | | X | X | X | X |

| | SCR^{a} | | | | | | | | | | | | |
|---|---|---|---|---|---|---|---|---|---|---|---|---|---|
| Procedure | (D-30 to D-2) | D-1 | D1 | D2 | D3 | D4 | D5 | D6 | D8 | D11 | D15 | D22 | D29 |
| Hematology (CBC) | X | X | X^{g} | X | X | | | | X | | Xⁿ | X | Xⁿ |
| Blood Chemistry | X | X | X^{g} | X | X | | | | X | | Xⁿ | X | Xⁿ |
| Urinalysis | X | X | X^{g} | X | X | | | | X | | Xⁿ | X | Xⁿ |
| Hepatitis Panel and HIV | X | | | | | | | | | | | | |
| QuantiFERON-TB Gold | X | | | | | | | | | | | | |
| Test | | | | | | | | | | | | | |
| RF and Anti-CCP | | X | | | | | | | | | | | |
| Drug and Alcohol Screen | X | X | | | | | | | | | | | |
| Blood Sample for | | | X^{p} | X | X | | X | | X | X | X^{q} | | X^{q} |
| ABBV-257 PK Assay^{o} | | | | | | | | | | | | | |
| Blood Sample for | X | | Xⁿ | | | | | | | | Xⁿ | | Xⁿ |
| ADA Assay^{o} | | | | | | | | | | | | | |
| Disease Response | | X^{r} | Xⁿ | | X | | X | | X | | Xⁿ | | Xⁿ |
| Biomarkers | | | | | | | | | | | | | |
| Blood Sample for | | | X | | | | | | | | | | |
| Pharmacogenetic | | | | | | | | | | | | | |
| Analysis^{s} | | | | | | | | | | | | | |

| | | | | | | | | | | | | | | PD |
|---|---|---|---|---|---|---|---|---|---|---|---|---|---|---|
| Procedure | D36 | D42 | D43 | D44 | D45 | D47 | D50 | D57 | D71 | D85 | D103 | D133 | D193 | Visit |
| Hematology (CBC) | X | | Xⁿ | | X | | X | X | X | | X | X | X | X |
| Blood Chemistry | X | | Xⁿ | | X | | X | X | X | | X | X | X | X |
| Urinalysis | X | | Xⁿ | | X | | X | X | X | | X | X | X | X |
| Hepatitis Panel and HIV | | | | | | | | | | | | | | |
| QuantiFERON-TB Gold | | | | | | | | | | | | | | |
| Test | | | | | | | | | | | | | | |
| RF and Anti-CCP | | | | | | | | | | | | | | |
| Drug and Alcohol Screen | | X | | | | | | | | | | | | |
| Blood Sample for | | | X^{t} | X | X | X | X | X | X | X | X | X | | X |
| ABBV-257 PK Assay^{o} | | | | | | | | | | | | | | |
| Blood Sample for | | | X^{u} | | | | X^{u} | X^{u} | X^{u} | X^{u} | X^{u} | X | X | X |
| ADA Assay^{o} | | | | | | | | | | | | | | |
| Disease Response | | | | | | | X | X | X | | X | X | | X |
| Biomarkers | | | | | | | | | | | | | | |
| Blood Sample for | | | | | | | | | | | | | | |
| Pharmacogenetic | | | | | | | | | | | | | | |
| Analysis^{s} | | | | | | | | | | | | | | |

| | SCR^{a} | | | | | | | | | | | | |
|---|---|---|---|---|---|---|---|---|---|---|---|---|---|
| Procedure | (D-30 to D-2) | D-1 | D1 | D2 | D3 | D4 | D5 | D6 | D8 | D11 | D15 | D22 | D29 |
| Pharmacodynamic | | X^{r} | Xⁿ | | X | | X | | X | | Xⁿ | | Xⁿ |
| (PD) Serum, Plasma, | | | | | | | | | | | | | |
| Biomarkers | | | | | | | | | | | | | |
| PD Biomarker - PBMC | | | Xⁿ | | | | | | | | Xⁿ | | Xⁿ |
| and mRNA | | | | | | | | | | | | | |
| PD Biomarker - | | | Xⁿ | | | | | | | | Xⁿ | | Xⁿ |
| Whole blood | | | | | | | | | | | | | |
| Fluorescence Optical | | | X | | | | | | | | | | |
| Imaging^{v} | | | | | | | | | | | | | |
| Complement Samples | | | X | | | | | | X | | X | X | X |
| (C3, C3a, C4), | | | | | | | | | | | | | |
| cytokines (TNF, IL-1β, | | | | | | | | | | | | | |
| IL-2, IL-6), | | | | | | | | | | | | | |
| and hsCRP^{w,x} | | | | | | | | | | | | | |
| hsCRP and ESR for DAS | | X | | | | | | | | | X | | X |
| Physician Global Disease | | X | | | | | | | | | X | | X |
| Activity VAS | | | | | | | | | | | | | |
| Patient Global Disease | | X | | | | | | | | | X | | X |
| Activity VAS | | | | | | | | | | | | | |

| | | | | | | | | | | | | | | PD |
|---|---|---|---|---|---|---|---|---|---|---|---|---|---|---|
| Procedure | D36 | D42 | D43 | D44 | D45 | D47 | D50 | D57 | D71 | D85 | D103 | D133 | D193 | Visit |
| Pharmacodynamic | | | | | | | X | X | X | | X | X | | X |
| (PD) Serum, Plasma, | | | | | | | | | | | | | | |
| Biomarkers | | | | | | | | | | | | | | |
| PD Biomarker - PBMC | | | | | | | | X | | | X | X | | X |
| and mRNA | | | | | | | | | | | | | | |
| PD Biomarker - Whole | | | | | | | | | | | X | X | | X |
| Blood | | | | | | | | | | | | | | |
| Fluorescence Optical | | | | | | | | X | | | | | | |
| Imaging^{v} | | | | | | | | | | | | | | |
| Complement Samples | X | | X | | | | X | | | | | | | |
| (C3, C3a, C4), | | | | | | | | | | | | | | |
| Cytokines (TNF, IL-1β, | | | | | | | | | | | | | | |
| IL-2, IL-6), | | | | | | | | | | | | | | |
| and hsCRP^{w,x} | | | | | | | | | | | | | | |
| hsCRP and ESR for DAS | | | X | | | | | X | | | X | X | | X |
| Physician Global Disease | | | X | | | | | X | | | X | X | | X |
| Activity VAS | | | | | | | | | | | | | | |
| Patient Global Disease | | | X | | | | | X | | | X | X | | X |
| Activity VAS | | | | | | | | | | | | | | |

| | SCR^{a} | | | | | | | | | | | | |
|---|---|---|---|---|---|---|---|---|---|---|---|---|---|
| Procedure | (D-30 to D-2) | D-1 | D1 | D2 | D3 | D4 | D5 | D6 | D8 | D11 | D15 | D22 | D29 |
| Swollen Joint Count | | X | | | | | | | | | X | | X |
| (SJC)/Tender Joint | | | | | | | | | | | | | |
| Count (TJC) | | | | | | | | | | | | | |
| Patient's Disease Pain VAS | | X | | | | | | | | | X | | X |
| HAQ-DI | | X | | | | | | | | | X | | X |
| Injection Site Assessment^{y} | | | | | | | | | | | | | |
| Urine Protein/Creatine Ratio | X | X | | | | | | | X | | Xⁿ | X | Xⁿ |
| 24-Hour Methylhistamine^{z} | | | | | | | | | | | | | |
| Tryptase^{x} | | | | | | | | | | | | | |
| Point of Care Assay | | | X^{aa} | | | | | | | | X^{bb} | | X^{bb} |
| (IL-6, IL-8) | | | | | | | | | | | | | |
| Adverse Event Assessment | | X | X | X | X | | X | | X | | X | X | X |

| | | | | | | | | | | | | | | PD |
|---|---|---|---|---|---|---|---|---|---|---|---|---|---|---|
| Procedure | D36 | D42 | D43 | D44 | D45 | D47 | D50 | D57 | D71 | D85 | D103 | D133 | D193 | Visit |
| Swollen Joint Count | | | X | | | | | X | | | X | X | | X |
| (SJC)/Tender Joint | | | | | | | | | | | | | | |
| Count (TJC) | | | | | | | | | | | | | | |
| Patient's Disease Pain VAS | | | X | | | | | X | | | X | X | | X |
| HAQ-DI | | | X | | | | | X | | | X | X | | X |
| Injection Site Assessment^{u} | | | | | | | | | | | | | | |
| Urine Protein/Creatine Ratio | X | | X^{k} | | | | X | X | X | | X | X | | X |
| 24-Hour Methylhistamine^{aa} | | | | | | | | | | | | | | |
| Tryptase^{s} | | | | | | | | | | | | | | |
| Point of Care Assay | | | X^{bb} | | | | | | | | | | | |
| (IL-6, IL-8) | | | | | | | | | | | | | | |
| Adverse Event Assessment | X | X | X | X | X | X | X | X | X | | X | X | X | X |

SCR = Screening;
D = Day;
PD = Premature Discontinuation
^{a}Perform within 30 days prior to study drug administration; procedures may be conducted under an IEC/IRB-approved screening consent.
^{b}Only the informed consent was obtained within 60 days of the first dose of study drug. Any screening procedures were performed within 30 days of the first dose of study drug.
^{c}Until completion of all study procedures on the scheduled day of discharge.
^{d}Subjects were required to stay at the site for at least 2 hours after dosing for safety monitoring.
^{e}Medical history update required.
^{f}Height at Screening only; symptom directed physical exam (PE) should be performed when necessary and if needed for physician assessments/questionnaires.
^{g}Predose (0 hr) measurements on Day 1 served as baseline for clinical assessment.
^{h}Vitals on Day 1 and Day 43 were collected at predose (0 hr) and 8 hours post dose.
ⁱA urine dipstick was sufficient for the urine pregnancy test.
^{j}Collected as a single tracing.
^{k}Collected as a triplicate tracing.
^{l}ECGs on Day-1 were collected in the afternoon. ECGs on Day 1 were collected predose (0 hr) and at 48, 96 and 168 hours post dose. ECGs post Day 43 were collected at 48, 96 and 168 hours post dose.
^{m}Study Visits 50-193 have a visit window of +/−2 days.
ⁿCollected prior to dose.
^{o}A visit window of ±2 days was added for all PK and ADA samples collected out of the confinement period.
^{p}Collected predose (0 hr) and at 8 hours post Day 1 dose.
^{q}Collected predose (0 hr), no more than 30 minutes before dose.
^{r}For enrolled subjects, sample was compared to predose value to assess intra-subject variability for each biomarker.
^{s}Optional: Subject signs a separate informed consent form (ICF); if the ICF was not signed, no pharmacogenetic sample was collected. The sample can be drawn at Day 1 or at any time during the subject's participation.
^{t}Collected predose (0 hr) and at 8 hours post Day 43 dose.
^{u}Collected predose (0 hr) and at 168, 336, 672, 1008 and 1440 hours post Day 43 dose.
^{v}Collected at Day-1 OR predose (0 hr) on Day 1. FOI is optional in subjects with tender/swollen joints in hand.
^{w}On Days 1, 43, and 50, is collected predose (0 hr) and at 2 and 6 hours post dose. On Days 15 and 29, is collected at predose (0 hr) and 2 hours post dose. On Days 8, 22 and 36, is collected once. All complements and hsCRP collected for all subjects in Group 1 and 2 were tested. Cytokine samples were archived and tested if necessary. If a subject within a dose group experiences a suspected hypersensitivity reaction or other systemic post-dose reaction, cytokine samples collected for all subjects dosed in that group were tested.
^{x}In the event of a suspected hypersensitivity reaction or other systemic post-dose reaction, additional blood samples were collected within 1, 3, and 24 hours of the onset of the reaction, if the reaction onset is within 48 hours of the subject's dose; samples at additional time points could be collected at the investigator's discretion..
^{y}Completed only in the event of an injection site reaction. De-identified pictures of injection sites were taken to aid in assessment of reaction.
^{z}In the event of a suspected hypersensitivity reaction or other systemic post dose reaction urine samples were collected once within 24 hours of the reaction, if the reaction onset is within 48 hours of the subject's dose.
^{aa}Collected at predose (0 hr) and at 1, 2, 4 and 8 hours post dose.
^{bb}Collected at predose (0 hr) and at 1 and 2 hours post dose.

### Study Procedures

### Medical History

A complete medical history, including alcohol, tobacco and nicotine-containing product use histories, was taken at Screening. The medical history was updated on Study Day—1. The medical history obtained at Screening served as the baseline for clinical assessment.

Medication (prescription or over-the-counter, including vitamins and herbal supplements) use from 30 days prior to study drug administration through the end of the study were recorded.

### Hepatitis Screen

All subjects were tested for the presence of the Hepatitis B Virus (HBV) and Hepatitis C Virus (HCV) at Screening. A positive result for the Hepatitis B surface antigen (HBs Ag) was exclusionary. Samples that were negative for HBs Ag were tested for HepB surface antibodies (HBsAb) and HepB core antibodies (HBcAb). If test results were positive for HBcAb or HBsAb, the patient had previously been infected with Hepatitis B and HBV, PCR was performed to determine if the patient was immune or whether the patient was still infected with Hepatitis B. Any result that met or exceeded detection sensitivity for HBV PCR was exclusionary as it was evidence of ongoing Hepatitis B infection. Patients with a positive Hepatitis C test, or with a past history of Hepatitis C infection were excluded. The hepatitis test panels were performed by a certified laboratory.

### HIV Screen

Subjects had their blood tested by a certified laboratory for the presence of anti-HIV Ab at Screening. Only those subjects negative for the presence of antibodies were allowed to enroll in the study. The results of the HIV Ab testing were retained by the study site under confidential restriction.

### QuantiFERON®-TB Gold Test

All subjects were tested for active or latent TB by the QuantiFERON®-TB Gold test. A subject that had a positive test was not allowed to enroll into the study. The results of the TB test were retained at the site as the original source documentation. Results were not transferred to the clinical database. The test was performed at Screening. The analyses were performed by a certified laboratory. Evidence of risk factors for latent TB were assessed by a questionnaire. The questionnaire may involve asking any number of questions. Exemplary questions include: Have you or an immediate family member or other close contact ever been diagnosed or treated for tuberculosis? Have you lived in or had prolonged travels to countries in the specific regions (Sub-Saharan Africa, India, China, Mexico, Southeast Asia or Micronesia, and the former Soviet Union); Have you lived or worked in a prison, homeless shelter, immigration center, or nursing home?; Have you, or an immediate family member, had any of the following problems for the past 3 weeks or longer: chronic cough; production of sputum, blood-streaked sputum, unexplained weight loss, fever, fatigue/tiredness, night sweats, and shortness of breath). These questions are exemplary and may be adapted, removed, or supplemented with additional questions. Only those subjects with a negative QuantiFERON®-TB Gold test and no evidence of latent TB risk factors were allowed to enroll in the study.

If the QuantiFERON®-TB Gold result was indeterminate, the test was repeated with a fresh blood sample. If a repeat QuantiFERON®-TB Gold result was indeterminate, this was considered a positive test result and the subject was excluded.

### Physical Examination

A physical examination was performed at time points specified in Table 19.

A symptom-directed physical examination was performed when necessary. Height was measured at Screening only. Body weight was measured at time points specified in Table 19.

The physical examination performed on Study Day-1 serves as the baseline physical examination for clinical assessment. Any significant physical examination findings after dosing were recorded as adverse events.

### Vital Signs

Body temperature, blood pressure and pulse were measured at time points specified in Table 19. The vital signs measurements just prior to dosing on Study Day 1 served as the baseline measurements for clinical assessment. Blood pressure and pulse rate were measured after the subject had been sitting for at least 3 minutes.

### 12-Lead Electrocardiogram (ECG)

A single 12-lead resting ECG was obtained at Screening; on Study Day 103, 133, and 193; upon subject premature discontinuation; or as clinically required. A 12-lead resting ECG was obtained in triplicate (approximately 2 minutes apart) as follows: Study Day—1 (in the afternoon); Study Day 1: pre dose (0 hr) and at 48, 96, and 168 hours post dose; and Study Day 43: 48, 96, and 168 hours post dose. The first of the triplicate ECG measurements obtained immediately prior to dosing on Study Day 1 served as the baseline for clinical assessment. When an ECG was scheduled at the same time as a blood collection, the ECG was obtained prior to the blood collection. ECGs occurring near meals took place prior to meals. ECGs were recorded after the subject had been in the supine position for at least 5 minutes. Subjects were instructed to remain completely stationary (no talking, laughing, deep breathing, sleeping, or swallowing) for approximately 10 seconds during the ECG recording. While ECGs were being acquired, subjects and staff were prohibited from having devices (e.g., cellular telephones, fans, heaters, etc.) that emit radiofrequency signals in the room.

### ECG Safety Review

Each ECG was printed and evaluated by an appropriately qualified physician (preferably a cardiologist) at the study site (the “local reader”). The local reading of the ECG was used by the investigator for subject safety assessments, including adverse event determination and management, dose escalation and decision on whether a subject was discontinued from the study.

ECGs designated as safety ECGs were interpreted, signed and dated by the local reader. Safety ECGs were: single ECGs (scheduled or unscheduled) and the first ECG of any triplicate set.

The local reader signed and dated the safety ECG and provided a global interpretation using the following categories: Normal ECG; Abnormal ECG—Not clinically significant (NCS); Abnormal ECG—Clinically significant (CS) and; Unable to evaluate.

All local reader evaluations of safety ECGs were entered into the source documents, electronic case report forms or paper case report forms. If the global interpretation was Abnormal (NCS or CS), the local reader provided further information (e.g., sinus bradycardia, arrhythmia) on the ECG worksheet. The QT interval corrected for heart rate using Fridericia's formula (QTcF) was documented on the worksheet only if the QT interval was determined to be prolonged by the local reader.

ECGs that were not designated as safety ECGs were evaluated by the local reader and recorded as “Assessed” then signed and dated. The evaluation was not entered into the case report form (electronic or paper) and the ECG worksheet, even if the local reader judged the ECG to be an Abnormal ECG—CS. However, an adverse event was recorded on the basis of a non-safety ECG that was judged to be an Abnormal ECG—CS.

All original ECG tracings were retained as source documentation in the subject's records at the study site. The automatic cardiograph reading (i.e., cardiograph-generated measurements and interpretations that were printed on the ECG tracing) was not collected for analysis.

### ECG Interval Collection and Measurement

The electronic tracings of all ECGs performed in triplicate and of any specified single ECGs were transferred to and evaluated by eECG/ABBIOS (electronic ECG/BIOsignal System), which uses a validated automated signal analysis algorithm to measure predefined ECG intervals (RR, PR, QT, and QRS duration).

A qualified Over Reader reviews the electronic ECG data using standardized quality-review criteria that were prospective, objective and evidence-based. All ECGs flagged according to the quality review criteria were manually adjudicated by the Over Reader, who inspected each flagged ECG and evaluated the accuracy of the interval measurements generated by eECG/ABBIOS. Based upon this manual verification, the Over Reader excluded the ECG from analysis or retained it for analysis, in which case the Over Reader adjusted or confirmed the measurements obtained by ABBIOS. The measurements obtained by the manual adjudication process superseded those initially obtained by eECG/ABBIOS. The data provided by eECG/ABBIOS were entered into the database and summarized.

### Screens for Drugs of Abuse and Alcohol

A urine screen for drugs of abuse was performed at time points specified in Table 19. The panel for drugs of abuse minimally includes cannabinoids, opiates, barbiturates, amphetamines, cocaine and benzodiazepines. These analyses were performed by the certified laboratory chosen for the study. Alcohol was prohibited from 48 hours prior to confinement and throughout the confinement period and was measured by a breath test. Drugs of abuse were prohibited throughout the study.

### Pregnancy Test

Urine and serum pregnancy tests were performed at time points specified in Table 19 for all female subjects. A dipstick test was sufficient for the urine pregnancy test. Serum pregnancy tests were performed by a certified laboratory.

### Clinical Laboratory Tests

Samples were obtained at a minimum for the clinical laboratory tests outlined in Table 19 and Table 20. Samples were obtained according to the time points specified in Table 19. The blood samples for serum chemistry tests were collected following a minimum 8-hour fast. For outpatient visits where samples for serum chemistry were collected, subjects fasted whenever possible. If a subject was not able to fast for serum chemistry due to unforeseen circumstances, the non-fasting status was recorded in the study source documentation.

A certified laboratory was utilized to process and provide results for the clinical laboratory tests. The baseline laboratory test results for clinical assessment for a particular test were defined as the last measurement prior to the initial dose of study drug.

TABLE 20
Clinical Laboratory Tests

Hematology
Hematocrit
Hemoglobin
Red blood cell (RBC) count
White blood cell (WBC) count
Neutrophils
Bands (if detected)
Lymphocytes
Monocytes
Basophils (if detected)
Eosinophils (if detected)
Platelet count (estimate not acceptable)
Mean corpuscular hemoglobin (MCH)
Mean corpuscular volume (MCV)
Mean corpuscular hemoglobin concentration (MCHC)
Prothrombin time (PT)
Activated partial thromboplastin time (aPTT)
Reticulocyte count
Clinical Chemistry
Blood urea nitrogen (BUN)
Creatinine
Total bilirubin
Albumin
Aspartate aminotransferase (AST)
Alanine aminotransferase (ALT)
Alkaline phosphatase
Sodium
Potassium
Calcium
Inorganic phosphorus
Uric acid
Cholesterol
Total protein
Glucose
Triglycerides
Bicarbonate/CO₂
Chloride
Lactate dehydrogenase (LDH)
High density lipoprotein (HDL) cholesterol
Low density lipoprotein (LDL) cholesterol
Very low density lipoprotein (VLDL) cholesterol
Magnesium
Gamma-glutamyl transpeptidase (GGTP)
Leucine aminopeptidase
Urinalysis
Specific gravity
Ketones
pH
Protein
Glucose
Blood
Urobilinogen
Bilirubin
Microscopic examination^{a}
Additional Labs^{b}
Urine Protein/Creatine Ratio
24-hour methylhistamine^{c}
Prothrombin time (PT)
Activated partial thromboplastin time (aPTT)
International Normalized Ratio (INR)
C3^{c}
C3a^{c} C4^{c}
Tryptase^{c}
High sensitivity CRP (hsCRP)^{c,d}
ESR^{d}
TNF^{c}
IL-1-β^{c}
IL-2^{c}
IL-6^{c}
HbA1c
RF
Anti-CCP
^{a}Only if abnormalities found in urinalysis.
^{b}As outlined in Table 19.
^{c}With occurrence of suspected hypersensitivity reaction or other systemic post-dose reaction, as outlined in Table 19.
^{d}Performed for DAS calculation.

For any laboratory test value outside the reference range that the investigator considered to be clinically significant:
The investigator repeated the test to verify the out-of-range value.
The investigator followed the out-of-range value to a satisfactory clinical resolution.
A laboratory test value that required a subject to be discontinued from the study or required a subject to receive treatment was recorded as an adverse event.
   Complement, Cytokine, hsCRP and Tryptase Measurements

For all subjects, samples for complement measurements (C3, C3a, and C4), cytokine measurements (TNF, IL-1β, IL-2 and IL-6), and high sensitivity CRP (hsCRP) were collected as follows:
On Days 1, 43, and 50, at predose (0 hr) and approximately 2 and 6 hours post injection. On Days 15 and 29, at predose (0 hr) and approximately 2 hours post injection. Once on Days 8, 22, and 36.

For Groups 1 and 2 only: All complements and hsCRP analyses were conducted for each subject; however, cytokine samples were archived (without analyses) for possible future evaluation if warranted.

For all other subjects in Group 3; all complement, hsCrp and cytokine samples were archived (without analysis) for possible future evaluation if warranted.

Samples for each dosing group were stored at the site under the direction of the site's laboratory, minimally until the last subject in the current dosing group has received their final dose. In the event of a suspected hypersensitivity reaction or other post-dose systemic reaction, all stable samples were analyzed. Once the last subject in the current dosing group had reached the time point of 30 days after the dose of study drug time point, samples for that group were destroyed pending sponsors approval.

If a subject experienced a suspected hypersensitivity reaction or other post-dose systemic reaction within 48 hours of the subject's dose, additional cytokine and complement samples, including hsCRP and a tryptase level, were collected from the subject(s) experiencing the reaction. If the reaction occurred while the subject was at the study site, these samples were collected within 1 hour, 3 hours and 24 hours after the onset of the reaction. If a subject experienced a suspected hypersensitivity reaction or other post-dose systemic reaction away from the study site, the subject notified the investigator and returned to the site for additional testing as soon as possible. Urine protein/creatine ratio and 24-hour methylhistamine were collected once within 24 hours after the onset of the reaction. Additional samples were collected as indicated thereafter to assist with characterizing the nature and etiology of the reaction. Based on the clinical status of the subject and in the opinion of the Investigator and/or Medical Monitor, additional sampling was considered.

### Point of Care Assay

Interleukins IL-6 and IL-8 were measured by a bedside (PicoScan) lateral flow immunoassay for semi-quantitative measurement at time points specified in Table 19. Additional assessments were scheduled according to the investigator's discretion.

### Injection Site Assessment

The injection site assessment was completed by the investigator for each subject only in the event of an injection site reaction. The assessment may involve a scale of grades 0 to 5. A zero grade may correspond to no pain or interference with activity; no treatment with non-narcotic or narcotic medications; no ER visit or hospitalization. The assessment might ask the patient how their experiences on a scale of 0 to 4. A grade of 1 indicates mild discomfort to touch, does not interfere with activity; erythema or induration of 2.5 to 5 cm. A grade of 2 indicates repeated use of non-narcotic pain reliever >24 hours or interferes with activity; discomfort with movement; erythema or induration of 5.1 to 10 cm. A grade of 3 indicates any use of narcotic pain reliever or prevents daily activity; significant discomfort at rest; erythema or induration of >10 cm. A grade 4 indicates an emergency room visit or hospitalization; necrosis or exfoliative dermatitis.

### Randomization and Assignment of Subject Numbers

The results of all screening and Study Day—1 evaluations were within clinically acceptable limits, upon review by the investigator before a subject can be administered study drug. Subjects were not enrolled in the study if laboratory or other screening results were unacceptable. Subjects who met the inclusion criteria and did not meet any of the exclusion criteria proceeded to randomization.

As they were enrolled into the study, subjects were assigned unique consecutive numbers and randomized.

### Confinement

Subjects were confined to the study site and supervised for periods of approximately 72 hours for first and last doses of study drug. Confinement for the first dose began on Study Day—1. Subjects remained at the study site and were supervised for at least 2 hours following the second and third doses of study drug. Confinement for the last dose began on Day 42. Each confinement period ended after the completion of all study procedures on the scheduled day of discharge.

### Meals and Dietary Requirements

Subjects received a standardized diet providing approximately 2400 kcal per day, for all meals (breakfast, lunch, dinner, snack) during confinement. During confinement, the subjects consumed only the scheduled meals provided in the study and water to quench thirst. The subjects abstained from all other food and beverage.

Subjects did not consume:
alcohol within the 48-hour period prior to confinement and during the confinement period, or
caffeine containing products or beverages during the confinement period.

### Blood Samples for Pharmacogenetic Analysis

One 4 mL whole blood sample for DNA isolation was collected on Day 1 from each subject who consented to provide samples for pharmacogenetic analysis. If the sample was not collected on Day 1, it was collected at any time throughout the study.

Whole blood was collected by standard phlebotomy techniques as described below:
Collect approximately 4 mL of blood into an appropriately labeled EDTA tube.
Immediately invest the collection tube 8 to 10 times to reduce the likelihood of clot formation.
Within 30 minutes of blood collection, store samples at −20° C. or colder until shipped on dry ice sufficient to last during transport.

### Drug Concentration and ADA Measurements

### Drug Concentration Sample Collection

The timing of blood collection was fundamental to the success of the study. The timing of blood collections took priority over all other scheduled study activities except for dosing. The order of blood collections was maintained to the minute such that the time intervals relative to the preceding dose were the same for all subjects. The time that each blood sample was collected was recorded to the minute. Blood samples for ABBV-257 assay were collected as closely as possible relative to the time of dosing according to the time points specified herein (e.g., Table 19). Subjects who dosed in the afternoon had their ABBV-257 blood samples drawn in the morning of the outpatient visits if necessary.

The blood samples were collected by venipuncture into appropriately labeled evacuated 10 mL serum collection tubes without gel separator. Sufficient blood was collected to provide approximately 1.5 mL serum from each sample. Blood was allowed to clot for 30 minutes at room temperature before centrifugation.

Twenty-one (21) blood samples were planned to be collected per subject for pharmacokinetic analysis. The total number of serum samples planned for pharmacokinetic analysis was 168 per group (a total of 504 samples for Groups 1 through 3 as planned).

### ADA Sample Collection

The serum samples for ADA assays were extracted from the serum collected from the 10 mL venipuncture draw for ABBV-257 (PK). Serum samples for the ADA assays were collected as outlined in Table 19.

Eleven (11) blood samples were planned to be collected per subject for ADA analysis. The total number of serum samples planned for ADA analysis was 88. A total of 264 samples for Groups 1 through 3 were planned.

### Handling/Processing of Samples

Blood Samples for Assays of ABBV-257 ADA

Blood samples for the ABBV-257 PK and ADA assays were centrifuged within 60 minutes of collection to separate the serum using a centrifuge.

At time points where only serum samples for ABBV-257 PK analysis were collected two 2 mL screw-capped polypropylene cryotubes were filled using plastic pipettes with approximately 0.75 mL of serum each. The residual serum volume can be disposed of.

At time points where ABBV-257 PK samples and ADA samples were collected, the total volume of serum derived from the 10 mL draw was equally split using plastic pipettes over 6 (approximately 0.75 mL per vial) 2 mL screw-capped polypropylene cryotubes.

The tubes were labeled with the drug number, type of sample (e.g., PK 1, PK 2, ADA 1, ADA 2, nADA 1 or nADA 2), type of matrix (e.g., serum), the protocol number, the subject number, and the planned time of sampling relative to dosing. The serum samples were frozen within 2 hours after collection and maintained at −20° C. (+/−5° C.) or colder until shipped.

Samples for the nADA assay were banked and analyzed upon request. ADA samples, nADA, and PK samples collected may also be used for assay development.

### Disposition of Samples

The frozen serum samples for the ABBV-257 and ADA assays were packed in dry ice sufficient to last during transport and shipped/transferred from the study site to the company site. An inventory of the samples included accompanied the package. Samples were batched and sent for all subjects within a group. The two splits of a sample set were shipped in separate shipments in case of transportation problems (e.g., custom, damage, or loss of shipment).

### Measurement Methods

Serum concentrations of ABBV-257 were determined and ADA analysis were performed using validated methods at the Drug Analysis Department.

### Safety Variables

The following safety evaluations were performed during the study: adverse event monitoring and vital signs, physical examination, ECG and laboratory tests assessments. The QT interval from the ECG were assessed with (QTc) and without correction. The QTc interval was calculated using the Fridericia correction.

### Pharmacokinetic Variables

The following pharmacokinetic parameters were estimated using non-compartmental methods: maximum observed serum concentration (Cmax), the time to Cmax (peak time, Tmax), the observed serum concentration at the end of the dosing interval (C trough) and the area under the concentration time curve at a dosing interval (AUCtau) for the first and the final dose intervals. The terminal phase elimination rate constant (β), the terminal elimination half-life (t1/2) and apparent clearance (CL/F) were determined after the final dose. In addition, accumulation ratios for Cmax and AUCtau from the first to the last dose were calculated.

If appropriate, other pharmacokinetic parameters were estimated. ADA titers were determined as part of the assessment of immunogenicity.

### Pharmacogenetic Variables

DNA samples were analyzed for genetic factors contributing to the subject's response to ABBV-257, or other study treatment, in terms of pharmacokinetics, efficacy, tolerability and safety. Such genetic factors may include genes for drug metabolizing enzymes, drug transport proteins, genes within the target pathway, or other genes believed to be related to drug response. Some genes currently insufficiently characterized or unknown may be important at the time of analysis. The samples were analyzed as part of a multi-study assessment of genetic factors involved in the response to ABBV-257 or drugs of this class. The samples were used for the development of diagnostic tests related to ABBV-257 (or drugs of this class). The results of pharmacogenetic analyses were exploratory and may not have been reported with the study summary.

### Pharmacodynamic Variables

The following clinical assessments of disease level were obtained for the time points specified in Table 19: swollen joint count, tender joint count, physician global assessment of disease activity using a visual analog scale (VAS), patient global assessment of disease activity using VAS, patient assessment of pain using VAS, HAQ-DI score, C-reactive protein (CRP), erythrocyte sedimentation rate (ESR), disease activity score 28 (both DAS28-CRP and DAS28-ESR) and American College of Rheumatology (ACR 20/50/70) response criteria.

### Tender Joint Count/Swollen Joint Count

### Tender Joint Count (TJC)

An assessment of 68 joints was done for tenderness by pressure manipulation on physical examination. Joint pain/tenderness was classified as either present (“1”), absent (“0”), replaced (“9”) or no assessment (“NA”). See Table 20.

### Swollen Joint Count (SJC)

An assessment of 66 joints was done by physical examination. The joints to be examined for swelling were the same as those examined for tenderness, except the hip joints were excluded. Joint swelling was classified as present (“1”), absent (“0”), replaced (“9”) or no assessment (“NA”). See Table 21 below.

TABLE 21
Tender Joint Count (TJC) and Swollen Joint Count (SJC) Example
JOINT EVALUATION

| | Patient Right | | | | | | Patient Left | | | | | |
|---|---|---|---|---|---|---|---|---|---|---|---|---|
| | | | | | 9 = | | | | | | 9 = | |
| | 0 = Absent | | | | Replaced | | 0 = Absent | | | | Replaced | |
| | 1 = Present | | | | NA = No | | 1 = Present | | | | NA = No | |
| JOINT | Pain/ | | | | Assessment | | Pain/ | | | | Assessment | |
| (Circle Correct Answer) | Tenderness | | Swelling | | Joint | | Tenderness | | Swelling | | Joint | |
| 1. Temporomandibular | 0 | 1 | 0 | 1 | 9 | NA | 0 | 1 | 0 | 1 | 9 | NA |
| 2. Sternoclavicular | 0 | 1 | 0 | 1 | 9 | NA | 0 | 1 | 0 | 1 | 9 | NA |
| 3. Acromio-clavicular | 0 | 1 | 0 | 1 | 9 | NA | 0 | 1 | 0 | 1 | 9 | NA |
| 4. Shoulder | 0 | 1 | 0 | 1 | 9 | NA | 0 | 1 | 0 | 1 | 9 | NA |
| 5. Elbow | 0 | 1 | 0 | 1 | 9 | NA | 0 | 1 | 0 | 1 | 9 | NA |
| 6. Wrist | 0 | 1 | 0 | 1 | 9 | NA | 0 | 1 | 0 | 1 | 9 | NA |
| 7. Metacarpophalangeal I | 0 | 1 | 0 | 1 | 9 | NA | 0 | 1 | 0 | 1 | 9 | NA |
| 8. Metacarpophalangeal II | 0 | 1 | 0 | 1 | 9 | NA | 0 | 1 | 0 | 1 | 9 | NA |
| 9. Metacarpophalangeal III | 0 | 1 | 0 | 1 | 9 | NA | 0 | 1 | 0 | 1 | 9 | NA |
| 10. Metacarpophalangeal IV | 0 | 1 | 0 | 1 | 9 | NA | 0 | 1 | 0 | 1 | 9 | NA |
| 11. Metacarpophalangeal V | 0 | 1 | 0 | 1 | 9 | NA | 0 | 1 | 0 | 1 | 9 | NA |
| 12. Thumb Interphalangeal | 0 | 1 | 0 | 1 | 9 | NA | 0 | 1 | 0 | 1 | 9 | NA |
| 13. Prox. Interphalangeal II | 0 | 1 | 0 | 1 | 9 | NA | 0 | 1 | 0 | 1 | 9 | NA |
| 14. Prox. Interphalangeal III | 0 | 1 | 0 | 1 | 9 | NA | 0 | 1 | 0 | 1 | 9 | NA |
| 15. Prox. Interphalangeal IV | 0 | 1 | 0 | 1 | 9 | NA | 0 | 1 | 0 | 1 | 9 | NA |
| 16. Prox. Interphalangeal V | 0 | 1 | 0 | 1 | 9 | NA | 0 | 1 | 0 | 1 | 9 | NA |
| 17. Distal Interphalangeal II | 0 | 1 | 0 | 1 | 9 | NA | 0 | 1 | 0 | 1 | 9 | NA |
| 18. Distal Interphalangeal III | 0 | 1 | 0 | 1 | 9 | NA | 0 | 1 | 0 | 1 | 9 | NA |
| 19. Distal Interphalangeal IV | 0 | 1 | 0 | 1 | 9 | NA | 0 | 1 | 0 | 1 | 9 | NA |
| 20. Distal Interphalangeal V | 0 | 1 | 0 | 1 | 9 | NA | 0 | 1 | 0 | 1 | 9 | NA |
| 21. Hip | 0 | 1 | — | — | 9 | NA | 0 | 1 | — | — | 9 | NA |
| 22. Knee | 0 | 1 | 0 | 1 | 9 | NA | 0 | 1 | 0 | 1 | 9 | NA |
| 23. Ankle | 0 | 1 | 0 | 1 | 9 | NA | 0 | 1 | 0 | 1 | 9 | NA |
| 24. Tarsus | 0 | 1 | 0 | 1 | 9 | NA | 0 | 1 | 0 | 1 | 9 | NA |
| 25. Metatarsophalangeal I | 0 | 1 | 0 | 1 | 9 | NA | 0 | 1 | 0 | 1 | 9 | NA |
| 26. Metatarsophalangeal II | 0 | 1 | 0 | 1 | 9 | NA | 0 | 1 | 0 | 1 | 9 | NA |

### Physician Global Assessment of Disease Activity

The physician globally assessed the subject's current RA activity utilizing a Visual Analog Scale (VAS). VAS was used to assess the subject's global assessment of disease activity. Each VAS consisted of a horizontal 100 mm line anchored at either end by opposite adjectives reflecting the spectrum/severity of the parameters assessed.

### Patient Global Assessment of Disease Activity

The subject assessed his/her overall rheumatoid arthritis disease activity within the past 24 hours utilizing a VAS. The subject completed the VAS before site personnel performed any clinical assessments and before any interaction with site personnel had occurred to avoid biasing the subject's response. Each VAS consisted of a horizontal 100 mm line anchored at either end by opposite adjectives (e.g., no pain and work possible pain) reflecting the spectrum/severity of the parameters assessed: Subject's global assessment of disease activity (within last 24 hours) The subject rated the severity of the RA symptoms and how he/she was doing from 0 to 100. This assessment was used for the DAS28 (CRP) calculation in this study.

For example, the patient was asked to place a vertical mark on the line below to indicate how well your rheumatoid arthritis has been doing during the last 24 hours (e.g., very well, very poorly, or somewhere in between).

### Patient's Assessment of Pain

The subject assessed his/her pain intensity for the past week utilizing a VAS. The subject completed the assessment before site personnel performed any clinical assessments and before any interaction with site personnel had occurred to avoid biasing the subject's response. VAS was used to for the subject's assessment of pain. Each VAS consisted of a horizontal 100 mm line anchored at either end by opposite adjectives reflecting the spectrum/severity of the parameters assessed. For example the patient was asked how much pain they had because of their condition within the previous week. The patient then placed a mark on a line (e.g., between no pain and worst possible pain) to indicate how severe their pain had been.

### HAQ-DI (Questionnaire)

The subject assessed his/her physical function during the past week using the HAQ-DI (e.g., questions regarding difficulties or ease in which he or she had been able to stand up, dress themselves, eat, walk, groom themselves, bend over, reach and get down a five pound object, grip a handle, open a jar, run errands, and get out of a car). The patient was asked to identify what aids or devices they had to use, e.g., cane, wheelchair, and long handled shoe horn. The subject completed the questionnaire before site personnel performed any clinical assessments and before any interaction with site personnel had occurred to avoid biasing the subject's response.

### DAS28 [CRP] Score and DAS [ESR] Score

The DAS28 [CRP] score was calculated using the following formula (DAS28-4(CRP) as given by (http://www.das-score.nl/):

DAS28[CRP]=0.56*sqrt(TJC28)+0.28*sqrt(SJC28)+0.36*ln(CRP+1)+0.014*GH+0.96

CRP refers to C-reactive protein expressed as mg/L. Sites were receiving the CRP from the laboratory in values in mg/dL.

GH refers to the Patient's Global Assessment of Disease Activity measured on a VAS of 100 mm.

TJC28 refers to the subject's tender joint count out of the provided 28 evaluated joints. SJC28 refers to the subject's swollen joint count out of the provided 28 evaluated joints.

The DAS28 [ESR] score was calculated using the following formula: DAS28 [ESR]=0.56*sqrt(TJC28)+0.28*sqrt(SJC28)+0.70*ln(ESR)+0.014*GH

TJC, SJC, and GH have the same definition as they do for DAS28 [CRP]. ESR was erythrocyte sedimentation rate.

### American College of Rheumatology Responder Criteria (ACR20/50/70)

For the American College of Rheumatology ACR20 responder criterion, a subject was classified as a responder if the following 3 criteria were met:
At least 20% improvement relative to baseline (Day—1) in TJC.
At least 20% improvement relative to baseline (Day—1) in SJC.
At least 20% improvement relative to baseline (Day—1) in 3 of the following 5 assessments:
   Patients assessment of pain (VAS)
   Patients global assessment of disease activity (VAS)
   Physician's global assessment of disease activity (VAS)
   Patients assessment of physical function (HAQ-DI score)
   Acute phase reactant value (CRP)
      The definition of ACR50 and ACR70 responders was the same as that of ACR20 except that 20% was replaced by 50% and 70%.

### Biomarkers

Blood samples were collected to assess the mechanism of action of ABBV-257 and disease response. Samples were analyzed for measurement of markers related to disease activity/prognosis of RA, autoimmunity/inflammation, and/or response to anti-RA medications, including ABBV-257 or drug of this class.

Pharmacodynamic and mRNA Biomarkers

Each subject had blood samples collected via venipuncture, prior to dosing (when applicable) at time points specified in Table 19. Separate instructions for the collection, handling and shipping of the pharmacodynamic serum, plasma, whole blood, PBMC and mRNA biomarkers were provided outside of the study protocol in the laboratory manual.

### Disease Response Biomarkers

Blood and urine samples were collected at time points specified in Table 19. The panel may include, but was not limited to: CRPM, MMP-3, C1M, C2M, C3M, CTX-I, CTX-II, osteocalcin and VICM. Separate instructions for the collection, handling and shipping of disease response biomarkers were provided outside of the study protocol in the laboratory manual. Due to diurnal variation, a urine sample from the second morning void was collected whenever possible.

### Pharmacodynamic Biomarkers

Blood samples were collected at time points specified in Table 19 to assess the mechanism of action of ABBV-257. Results from these exploratory studies were necessarily be a part of the study report. Disease Response Biomarkers Subjects have additional blood samples collected at time points specified in Table 19 and to assess disease response. Samples were stored frozen for future measurement of non-genetic markers related to disease activity/prognosis of RA, autoimmunity/inflammation, and/or response to anti-RA medications, including_ABBV-257 or drug of this class.

### Fluorescence Optical Imaging (FOI)

FOI is a non-invasive exploratory optical imaging methodology which images the microcirculation in the joints of the hands and wrists assessing individual disease activity and treatment response in patients with RA.

The evaluation utilizing FOI was optional. FOI analysis was conducted in subjects demonstrating at least one tender/swollen joint in either hand or wrist prior to study treatment. FOI was an exploratory imaging procedure used to potentially detect early pharmacodynamic effects of ABBV-257. FOI procedure consists of IV administration of indocyanine green (0.1 mg/kg) followed by image acquisition for about 10 minutes with commercially available FOI system.

Subjects consented to this procedure by signing the IEC/IRB approved informed consent form (ICF). Further details regarding the specific imaging techniques were provided outside of the study protocol by the sponsor or designee.

Removal of Subjects from Therapy or Assessment

### Discontinuation of Individual Subjects

Each subject had the right to withdraw from the study at any time. In addition, the investigator discontinued a subject from the study at any time if the investigator considers it necessary for any reason, including the occurrence of an adverse event or noncompliance with the protocol. Subjects who withdrew from the study were not replaced unless it was mutually agreed upon, in writing, by the investigator and the company.

In the event that a subject withdrew or was discontinued from the study, the primary reason for discontinuation and any other reason(s) for the discontinuation from the study were recorded and a physical examination, body weight, vital signs measurement, ECG, laboratory analyses, biomarker collections, disease response assessments and an assessment of adverse events were performed as soon as possible after discontinuation from the study. Additional blood samples for drug measurement were collected at the time of discontinuation from subjects who were discontinued due to adverse events; the clock time, time in relation to dose, and date the sample was taken were recorded.

If a subject was discontinued from the study with an ongoing adverse event or an unresolved laboratory result that was significantly outside of the reference range, the investigator attempted to provide follow-up until a satisfactory clinical resolution of the laboratory result or adverse event was achieved.

In the event that a positive result was obtained on a pregnancy test for a subject or a subject reported becoming pregnant during the study, the administration of study drug to that subject was discontinued immediately. The investigator reported a pregnancy within 1 working day of the site being aware to one of the company representatives.

### Discontinuation of Entire Study

The company may terminate this study prematurely, either in its entirety or at any study site, for reasonable cause provided that written notice was submitted in advance of the intended termination. The investigator may also terminate the study at his/her site for reasonable cause.

### Dose Escalation and Stopping Criteria

Dose escalation was reevaluated and adjusted, dosing suspended, or the study potentially stopped, should one or more of the following occur within a dosing group:

### Adverse Events

Two or more subjects who received active study drug experience moderate to severe adverse events that were of a similar nature and for which the investigator considers the relationship to study drug as a reasonable possibility. However, AEs not directly related to study drug administration such as vasovagal syncope or phlebitis due to blood draws should generally not be considered.
A subject who received active study drug experiences a serious adverse event for which the investigator considered the relationship to study drug to be a reasonable possibility.

### Post-Dose Systemic Hypersensitivity Reaction

One or more subjects (who received active study drug) experienced a serious systemic hypersensitivity reaction.

### Injection Site Reactions

Two or more subjects (who received active study drug) experienced a moderate to severe injection site reaction.

### Laboratory Abnormalities, ECG Changes

Two or more subjects on active drug experience a confirmed WBC <2000 cells/mm³
Two or more subjects on active drug experienced a confirmed ANC <1000 cells/mm³
Two or more subjects on active drug experienced a confirmed haemoglobin <8 gm/dL WITH a decline of at least 1.5 gm/dL or more from baseline.
Two or more subjects on active drug experienced a confirmed platelet count <50,000 cells/mm³.
Two or more subjects on active drug experienced a confirmed creatinine >2×ULN.
One subject who received active study drug having confirmed ALT or AST >3×ULN and total bilirubin >2×ULN or INR >1.5, for which no alternative etiology was identified.
At least one subject administered ABBV-257 having confirmed ALT or AST >3×ULN with the appearance of fatigue, nausea, vomiting, right upper quadrant pain or tenderness, fever, rash, and/or eosinophilia (>5%), for which no alternative etiology was identified.
One subject who received active study drug with ECG changes that were considered clinically significant for which the investigator considered the relationship to study drug to be a reasonable possibility or an absolute QTcF value >500 msec for any scheduled ECGs.

Dosing was suspended in individual subjects if any of the following occur:
A subject experienced a serious adverse event (SAE) for which the investigator considers the relationship to study drug to be a reasonable possibility.
A subject experienced a severe injection site reaction.
A subject had a confirmed absolute neutrophil count (ANC)<1000 cells/mm³ or WBC <2000 cells/mm³
A subject had a confirmed platelet count <50,000 cells/mm³
A subject had a confirmed hemoglobin <8.0 gm/dL WITH a decline of at least 1.5 gm/dL or more from baseline.
A subject had a confirmed rise in creatinine of 50% above baseline.
A subject had a confirmed ALT or AST >3×ULN with a Total Bilirubin >2×ULN or INR >1.5.
A subject had a confirmed ALT or AST >3×ULN with the appearance of fatigue, nausea, vomiting, right upper quadrant pain or tenderness, fever, rash, and/or eosinophilia (>5%).
A subject had a confirmed ALT or AST >8×ULN.
A subject experienced a confirmed ECG change considered clinically significant for which the investigator considers the relationship to study drug to be a reasonable possibility OR a confirmed absolute QTcF value >500 msec for any scheduled ECGs.
A subject had a nonserious AE of a systemic hypersensitivity reaction with clinical symptoms involving 2 or more body systems as well as a detectable ADA response and ABBV-257 levels below the level of detection documented prior to the onset of the AE.

### Treatments

### Treatments Administered

Study drug was administered as follows in Table 22:

TABLE 22
Dosing Schedule

| Group | Dose | Dosing Days |
|---|---|---|
| 1 | 30 mg SC | 1, 15, 29, 43 |
| 2 | 100 mg SC | 1, 15, 29, 43 |
| 3 | 300 mg SC | 1, 15, 29, 43 |

Dosing for Groups 2 and 3 was enabled after all subjects in the previous group have satisfactorily completed at least a minimum of 1 week of safety assessments after the last subject's second dose was administered. The escalation scheme was adjusted (e.g., dosing interval, number of doses) based on PK and safety from preceding dose groups based on data from previous group(s). The number of injections per subject varied by dose level. Depending on the number of syringes required for each subject, one injection per site was administered subcutaneously in the following order (as needed):

1. Left upper quadrant of the abdomen

2. Right upper quadrant of the abdomen

3. Left anterior proximal thigh

4. Right anterior proximal thigh

5. Left lower quadrant of the abdomen

6. Right lower quadrant of the abdomen

The areas to avoid for SC injections included: any blood vessels, thickening or t tenderness of skin, scars, fibrous tissue, lesions, stretch marks, bruises, redness, nevi, or other skin imperfections. Injection sites were at least 1 inch apart and at least 2 inches from the navel. The subject remained in a supine position for at least 30 minutes following study drug administration. The time of each drug administration was recorded to the nearest minute.

### Identity of Investigational Products

Information about the ABBV-257 formulations used in this study is presented i n Table 23.

TABLE 23
Identity of Investigational Products

| Investiga- | ABBV-257 | Placebo for ABBV-257 |
|---|---|---|
| tional | 50 mg Powder for | 50 mg Powder for |
| Product | Injection Vial | Injection Vial |
| Dosage form | Powder for solution for | Powder for solution for |
| | injections in vials | injection in vials |
| Strength | 50 mg/mL when | N/A |
| (mg) | reconstituted with 1.2 | |
| | mL of sterile water | |
| | for injection | |
| Mode of | Subcutaneous injection | Subcutaneous injection |
| Administration | | |

ABBV-257 50 mg powder for solution for injection vial and matching placebo for

ABBV-257 50 mg powder for solution for injection vial were reconstituted with sterile water for injection described herein.

### Packaging and Labeling

Study drug ABBV-257 50 mg powder for solution for injection vials and matching placebo for ABBV-257 50 mg powder for solution for injection vial was open-labeled and packaged in cartons. Each vial and carton included at least the information as required by local regulations. Each label remain affixed to the vial and carton.

Study drug was provided as a powder in vials that were reconstituted to a solution for subcutaneous injection at the study site by the unblinded pharmacist or designee prior to dosing.

ABBV-257 active and placebo vials were packed separately in kit cartons.

Once vials were prepared for subcutaneous injection, each prepared syringe was labeled with a syringe label to be completed by the unblinded study drug preparation designee or pharmacist. No information was provided on the label that breaks the study blind.

### Storage and Disposition of Study Drugs

ABBV-257 vials and matching placebo vials must be stored at 2° to 8° C./36° to 46° F., protected from light, and must not be frozen. A storage temperature log was maintained to document proper storage conditions. The refrigerator temperature was recorded on a daily basis (excluding weekend/holidays) on temperature log to record proper function. Malfunctions or any temperature excursion must be reported to immediately. Study drug was quarantined and not dispensed until GPRD deems the medication as acceptable.

All study drug (dispensed, used, or unused) was handled at the study site by the assigned site personnel. The unblinded study monitor was responsible for inventory and plans for final disposition of study drug. The investigational products were for investigational use only and were to be used only within the context of this study. The study drug supplied for this study was maintained under adequate security to prohibit unblinding. The study drug was stored under the conditions specified on the label until dispensed for subject use or returned to the destruction facility.

### Preparation/Reconstitution of Dosage Forms

The ABBV-257 drug product (active and placebo) was provided as a powder in vials. Each vial of ABBV-257 and placebo was reconstituted with 1.2 mL of sterile water for injection to provide a 50 mg/mL ABBV-257 active or a placebo solution. The ABBV-257 drug product was dosed as a fixed dose. The total volume administered was dependent upon the assigned dose. The ABBV-257 drug product and placebo solutions were administered via subcutaneous (SC) injection. Specific dose preparation and documentation details were provided to the site pharmacy outside of this protocol.

### Method of Assigning Subjects to Treatment Groups

The randomization schedule was computer-generated before the start of the study by the Statistics Department. As they were randomized in the study, subjects of each group were assigned unique, consecutive numbers beginning with 1001 for Group 1, 2001 for Group 2 and 3001 for Group 3. Within each group, subjects were randomized in a 3:1 ratio to receive either ABBV-257 or matching placebo. If additional groups were added (beginning with Group 4), subjects of each group were assigned unique, consecutive numbers (beginning with 4001) for randomization to ABBV-257 or matching placebo.

### Selection and Timing of Dose for Each Subject

Selection of the dose for this study was discussed herein. Within a group, the subjects assigned to ABBV-257 were administered the same dose. ABBV-257 or matching placebo were administered on Study Days 1, 15, 29, and 43 prior to MTX dose.

### Blinding

The study was conducted in a double-blind manner such that the principal investigator, study coordinator, subjects and the study team were blinded to the treatment assignments. Placebo in its powder form was identical in appearance to the ABBV-257 powder form; however, both were delivered to the study drug preparation designee or pharmacist in an open-label format for further preparation.

The study designated statisticians assigned to this study were unblinded to allow for expedited review of the safety and pharmacokinetic data.

### Appropriateness of Measurements

Standard pharmacokinetic, statistical, clinical, and laboratory procedures were utilized in this study. Disease response, biomarker and imaging data were collected for exploratory analysis.

### Suitability of Subject Population

This study enrolls male and female subjects who have been diagnosed with RA and have been on MTX for at least 3 months and were on a stable regimen of MTX (7.5-25 mg/week) for at least 4 weeks. The study population selected in this study reflects the standard population for RA trials with new intervention.

### Selection of Doses in the Study

ABBV-257 has been evaluated in the first-in-human (FIH) single ascending dose study (Study M14-355) in healthy subjects that included IV doses of 0.3, 1.0 and 3.0 mg/kg and SC doses of 0.3 and 3 mg/kg. This study recently completed dosing; preliminary analysis of safety data demonstrate a favorable safety profile up to 3 mg/kg following IV or SC administration. There were no severe or serious adverse events following study treatment. No subjects prematurely discontinued due to adverse events to ABBV-257. All infections were mild in severity and no systemic hypersensitivity reactions were reported. Study M14-355 safety data were reviewed more in detail at ABBV-257 investigator's brochure.

When ABBV-257 was tested in a GLP 8-week monkey toxicology study, there were no first-dose infusion reactions at any of the dose levels tested (60, and 200 mg/kg IV and 200 mg/kg SC), nor any reactions observed with the SC route of administration. The NOAEL dose was 200 mg/kg IV and resulted in a Cₘₐₓ and estimated AUC_{0-14day} of 14,600 μg/mL and 147,500 μg·day/mL, respectively. The estimated AUC at the NOAEL provide 1222- and 122-fold safety margin relative to the steady-state AUC at the starting dose of 30 mg/kg EOW and highest dose of 300 mg/kg EOW, respectively. In addition, the preliminary estimate of AUC_{inf} at the highest IV dose in the SAD study provide 1.3-fold margin from the predicted steady-state exposure at the highest dose proposed for this study.

ABBV-257 is a high-affinity bispecific recombinant human molecule with TNF-binding properties comparable to those of the monoclonal anti-TNF antibody Adalimumab. Affinities for TNF were 5 pM with ABBV-257, 8 pM with a distinct TNF/IL-17 DVD-Ig and 30 pM with Adalimumab. In a Phase 1 clinical trial of patients with RA administered a single IV dose of Adalimumab, a clinical response was observed at the lowest dose tested, 0.5 mg/kg, with greater response observed at all higher doses tested, up to 10 mg/kg. In a Phase 3 study of multiple, SC doses of Adalimumab monotherapy for RA, a successively greater clinical response was observed with increasing exposure in doses corresponding to a dose range of approximately 0.3 to 1.14 mg/kg. This range encompasses the indicated starting dose for Adalimumab in RA, 40 mg every other week (EOW), which corresponds to approximately 0.6 mg/kg.

### Adverse Events

The investigator monitored each subject for clinical and laboratory evidence of adverse events on a routine basis throughout the study. The investigator assessed and record any adverse event in detail including the date of onset, event diagnosis (if known) or sign/symptom, severity, time course (end date, ongoing, intermittent), relationship of the adverse event to study drug, and any action(s) taken. For serious adverse events considered as having “no reasonable possibility” of being associated with study drug, the investigator provided another cause of the event. For adverse events to be considered intermittent, the events must be of similar nature and severity.

Adverse events, whether in response to a query, observed by site personnel, or reported spontaneously by the subject were recorded. All adverse events were followed to a resolution.

### Adverse Event

An adverse event was defined as any untoward medical occurrence in a patient or clinical investigation subject administered a pharmaceutical product and which does not necessarily have a causal relationship with this treatment. An adverse event therefore was any unfavorable and unintended sign (including an abnormal laboratory finding), symptom, or disease temporally associated with the use of a medicinal (investigational) product, whether or not the event was considered causally related to the use of the product.

Such an event can result from use of the drug as stipulated in the protocol or labeling, as well as from accidental or intentional overdose, drug abuse, or drug withdrawal. Any worsening of a pre-existing condition or illness was considered an adverse event. Worsening in severity of a reported adverse event was reported as a new adverse event. Laboratory abnormalities and changes in vital signs were considered to be adverse events only if they result in discontinuation from the study, necessitate therapeutic medical intervention, and/or if the investigator considers them to be adverse events.

An elective surgery/procedure scheduled to occur during a study was not be considered an adverse event if the surgery/procedure was being performed for a pre-existing condition and the surgery/procedure has been pre-planned prior to study entry. However, if the

pre-existing condition deteriorates unexpectedly during the study (e.g., surgery performed earlier than planned), then the deterioration of the condition for which the elective surgery/procedure was being done was considered an adverse event.

### Serious Adverse Events

If an adverse event met any of the following criteria, it was to be reported as a serious adverse event within 24 hours of the site being made aware of the serious adverse event:

### Death of Subject

An event that results in the death of a subject.

### Life-Threatening

An event that, in the opinion of the investigator, would have resulted in immediate fatality if medical intervention had not been taken. This does not include an event that would have been fatal if it had occurred in a more severe form.

### Hospitalization or Prolongation of Hospitalization

An event that resulted in an admission to the hospital for any length of time or prolongs the subject's hospital stay. This does not include an emergency room visit or admission to an outpatient facility.

### Congenital Anomaly

An anomaly detected at or after birth, or any anomaly that results in fetal loss.

### Persistent or Significant Disability/Incapacity

An event that resulted in a condition that substantially interferes with the activities of daily living of a study subject. Disability was not intended to include experiences of relatively minor medical significance such as headache, nausea, vomiting, diarrhea, influenza, and accidental trauma (e.g., sprained ankle).

Important Medical Event Requiring Medical or Surgical Intervention to Prevent Serious Outcome

An important medical event that may not be immediately life-threatening or resulted in death or hospitalization, but based on medical judgment may jeopardize the subject and may require medical or surgical intervention to prevent any of the outcomes listed above (i.e., death of subject, life-threatening, hospitalization, prolongation of hospitalization, congenital anomaly, or persistent or significant disability/incapacity). Additionally, any elective or spontaneous abortion or stillbirth was considered an important medical event. Examples of such events include allergic bronchospasm requiring intensive treatment in an emergency room or at home, blood dyscrasias or convulsions that do not result in inpatient hospitalization, or the development of drug dependency or drug abuse.

### Relationship to Study Drug

The investigator uses the following definitions to assess the relationship of the adverse event to the use of study drug:

### Reasonable Possibility

An adverse event where there was evidence to suggest a causal relationship between the study drug and the adverse event.

### No Reasonable Possibility

An adverse event where there was no evidence to suggest a causal relationship between the study drug and the adverse event.

For causality assessments, events assessed as having a reasonable possibility of being related to the study drug were considered “associated.” Events assessed as having no reasonable possibility of being related to study drug were considered “not associated.”

In addition, when the investigator has not reported a causality or deemed it not assessable, the company considers the event associated.

If an investigator's opinion of no reasonable possibility of being related to study drug was given, another cause of event must be provided by the investigator for the serious adverse event.

### Adverse Event Collection Period

All adverse events reported from the time of study drug administration until 5 half-lives following discontinuation of study drug administration have elapsed were collected, whether solicited or spontaneously reported by the subject. In addition, serious adverse events and protocol-related nonserious adverse events were collected from the time the subject signed the study-specific informed consent.

During the outpatient portion(s) of the study, subjects were provided with a telephone number for the study site and instructions to contact the study site if they experience an adverse event requiring medical care. Adverse event and medical history information were updated upon subject reconfinement to confirm eligibility for continued participation in the study.

### Adverse Event Reporting

In the event of a serious adverse event, whether associated with study drug or not, the Investigator notifies Clinical Pharmacovigilance within 24 hours of the site becoming aware of the serious adverse event by entering the serious adverse event data into the electronic data capture (EDC) system. Serious adverse events that occur prior to the site having access to the RAVE® system or if RAVE was not operable should be faxed to Clinical Pharmacovigilance within 24 hours of being made aware of the serious adverse event.

Statistical Methods and Determination of Sample Size

Statistical and Analysis Plans

All hypothesis testing was performed at significance level of two-tailed 0.05 unless specified otherwise.

Demographics and Baseline Characteristics

Descriptive statistics were provided for demographic variables and baseline characteristics with a breakdown by dose level. The data of the subjects assigned to placebo were combined.

Pharmacokinetics

Tabulations and Summary Statistics

Serum concentrations of ABBV-257 and pharmacokinetic parameter values were tabulated for each subject and each regimen as defined by dose level and frequency of dosing, and summary statistics were computed for each sampling time and each parameter by regimen. ADA assay data was also tabulated by subject and descriptive statistics were provided for each sampling time by regimen.

Model and Tests

To investigate attainment of steady state, a repeated measures analysis is performed on the trough concentration measurements for the second through last times of dosing. The logarithmic transformation was used unless the data show good reason to do otherwise (e.g., measurements below the lower limit of quantification or a skewness coefficient with magnitude >1.0) while untransformed Ctrough or another transformation had an approximate symmetric distribution. The model has classification by dose level and day and includes an effect for the interaction of dose level and day. If the statistic on the interaction of dose level and day is not significant at level 0.05, inferences are based upon the main effects for day (the average over the several dose levels). If the statistic for the interaction is significant at level 0.05, inferences are made for each dose level within the framework of the model. For each time of measurement before the last dose, a point estimate and 95% confidence interval are provided for the ratio of the central value on that day to the central value on the day of the last dose. Assuming that the logarithm was analyzed, the point estimate and confidence limits for a ratio were obtained by exponentiation of the point estimate and confidence limits for the difference of logarithm means.

To address the issue of dose proportionality and linear kinetics after multiple doses, an analysis is performed on the pharmacokinetic parameters. The parameters include dose-normalized Cmax, dose normalized AUC, and dose normalized Ctrough of the last dose interval and β after the last dose. For this analysis, Ctrough is the concentration at the end of the dose interval. An analysis is performed for each pharmacokinetic parameter in turn. Observations are classified by dose level. For dose-normalized Cmax and dose normalized AUC, the logarithmic transformation is employed unless the data give evidence that the logarithm has considerable nonsymmetry (e.g., skewness coefficient with magnitude ≧1.0). For dose-normalized Ctrough, the transformation decided upon for the analysis of change with time is used for this analysis also. For the exposure variables, body weight is a covariate. Other variables that might explain some of the variability among subjects may be considered. Except for body weight for an exposure variable, a necessary condition for a variable to be included in the final model is that the regression coefficient be significant at level 0.10. Within the framework of the final model, the hypothesis of no difference between the highest and lowest dose levels is tested.

An analysis is performed to estimate the accumulation ratio for C max and AUC from the first dose to the last dose of the regimen. If the test statistic on the dose level effect is not significant at level 0.05, the estimate of the accumulation ratio is the exponentiation of the estimate of the average of the mean changes for the several dose levels. If the test statistic on dose level effect is significant, an estimate of the accumulation ratio is provided for each dose level separately. Along with the point estimate, a 95% confidence interval is provided within the framework of the ANOVA. Additional analyses are performed if useful and appropriate.

Safety

If subjects prematurely discontinue from the study for reasons that were possibly study drug related, the possibility of bias in the estimation of drug effects due to the resulting missing data was addressed.

The data of the subjects assigned to placebo re combined. Interval measurements from triplicate ECGs are averaged to obtain a single value for each time of measurement. The QTc interval is calculated using the Fridericia correction.

For laboratory variables and vital signs, the baseline value is the last measurement obtained before the first dose of the study drug which is expected to be a measurement obtained prior to dosing on the day of the first dose. For ECG variables, the baseline value is the average from the predose ECG on the day of the first dose and the ECG on Day—1.

Adverse events were coded using Medical Dictionary for Regulatory Activities (MedDRA). The number and percentage of subjects reporting treatment-emergent adverse events (onset or worsening after first dose of study drug administration) were tabulated by MedDRA preferred term and system organ class with a breakdown by dose level. Tabulations were also be provided in which the number of subjects reporting an adverse event (MedDRA term) was additionally broken down by rating and by whether the event was associated with the study drug. Any deaths, other serious adverse events and other significant adverse events, including those leading to treatment discontinuation, were also separately identified.

The serious and nonserious adverse events occurring after the study specific informed consent was signed but prior to the first drug of the investigational product that considered to be causally related to study required procedures are summarized.

Laboratory test values and measurements on vital signs that were potentially clinically significant, according to predefined criteria, are identified. ECG QT and QTc interval values >500 msec and changes from baseline >30 msec and >60 msec are identified.

Descriptive statistics were provided for blood pressure, pulse rate, quantitative ECG variables and laboratory variables for each scheduled time of measurement with a breakdown by dose level. A repeated measures analysis was performed on the scheduled measurements of the last dose interval, beginning with the first post-dose measurement and ending with the measurement on Day 70. For this analysis, the data of subjects administered placebo was combined; i.e., there was a single placebo group for the purposes of this analysis. A statistical analysis that includes measurements before or after the last dose interval was performed if the analysis for the last dose interval gives evidence of an effect of ABBV-257, if the descriptive statistics suggest an effect of ABBV-257 before the last dose interval, or if there was other reason to do so. In particular, an analysis that includes data obtained before the last dose interval was considered if there are subjects who prematurely terminate for reasons possibly related to ABBV-257, with the possibility that the absence of data from these subjects in the last dose interval has resulted in meaningful bias.

For the repeated measures analysis, the model has an effect for baseline value, classification by dose level (with placebo considered a dose level of zero) and by day of measurement and have an effect for the interaction of dose level with day of measurement. The model has an appropriate structure for the variance/covariance matrix for the measurements from a subject.

Within the framework of the model, the estimate of the difference in mean from placebo is provided for each ABBV-257 dose level for each time of measurement, and the results of a test on each of these differences is reported. However, the primary emphasis is on a test that has good power for an effect of ABBV-257 that is an approximately linear function of the anticipated ABBV-257 exposure or of the logarithm of anticipated exposure and on the test for the comparison of the with highest dose level of ABBV-257 to placebo. These tests are performed on the dose level main effects (pertaining to an average over the several times of measurement) and also for each time of measurement, but all within the framework of the repeated measures analysis. Whether the focus is on the tests for the individual times of measurement or on the tests on the dose level main effects are depend upon whether the statistic on the interaction of dose level and day of measurement is significant at level 0.10.

Additional analyses are performed if useful and appropriate.

### Pharmacodynamics

The baseline value for a pharmacodynamic variable is the value obtained prior to the first dose of the study drug. For the clinical assessment variables that are quantitative (as opposed to categorical), descriptive statistics are provided for each scheduled time of assessment after the beginning of the regimen. This includes descriptive statistics for the change from baseline, as well as for the given time of assessment. A repeat measures analysis is performed. Observations are classified by dose level, and the baseline value is the covariate. For this analysis, the data of subjects administered placebo are combined; i.e., there is a single placebo group for the purposes of this analysis. If it may be useful, an analysis is performed for earlier times of assessment. If a variable appears to have notable skewness (e.g., skewness coefficient >1.5), a transformation is sought in order to meaningfully reduce the degree of non-symmetry. If a variable appears to have a distribution with both tails quite long, a simple nonparametric analysis may be performed.

Within the framework of the model, the estimate of the difference in mean from placebo is provided for each ABBV-257 dose level, and the results of a test on each of these differences are reported. However, the primary emphasis is on a test that has good power for an effect of ABBV-257 that is an approximately linear function of the anticipated ABBV-257 exposure or of the logarithm of anticipated exposure and on the test for the comparison of the highest ABBV-257 dose level to placebo.

For each of the ACR responder criteria, the number and percentage of subjects who satisfy the criterion are tabulated by day of assessment and dose level. For these tabulations the placebo data are combined.

The biomarker data are summarized as appropriate. Additional analyses may be performed if useful and appropriate.

Without being limited by any particular theory or mechanism of action, it is here envisioned that patient data shows that ABBV-257 DVD-Ig binding protein effectively treats rheumatoid arthritis in patients, and rheumatoid arthritis patients that are resistant to other therapeutic agents, e.g., DMARDs.

### INCORPORATION BY REFERENCE

The present invention incorporates by reference in their entirety techniques well known in the field of molecular biology, drug delivery, immunology, molecular biology and cell biology. These techniques include, but are not limited to, techniques described in the following publications: Ausubel et al. (eds.) (1993) Current Protocols in Molecular Biology, John Wiley & Sons, NY; Ausubel et al. (eds.) (1999) Short Protocols In Molecular Biology John Wiley & Sons, NY (ISBN 0-471-32938-X); Smolen and Ball (eds.) (1984) Controlled Drug Bioavailability Drug Product Design and Performance, Wiley, NY; Giege and Ducruix (1999) Crystallization of Nucleic Acids and Proteins, a Practical Approach, 2nd ed., pp. 20 1-16, Oxford University Press, NY; Goodson (1984) Medical Applications of Controlled Release, vol. 2, pp. 115-138; Hammerling et al. (1981) Monoclonal Antibodies and T-Cell Hybridomas 563-681 (Elsevier, N.Y.; Harlow et al. (1988) Antibodies: A Laboratory Manual, (Cold Spring Harbor Laboratory Press, 2nd ed.; Kabat et al. (1987) Sequences of Proteins of Immunological Interest (National Institutes of Health, Bethesda, Md.; Kabat et al. (1991) Sequences of Proteins of Immunological Interest, Fifth Edition, U.S. Department of Health and Human Services, NIH Publication No. 91-3242; Kontermann and Dubel (eds.) (2001) Antibody Engineering Springer-Verlag, NY 790 pp. (ISBN 3-540-41354-5); Kriegler (1990) Gene Transfer and Expression, A Laboratory Manual, Stockton Press, NY; Lu and Weiner (eds.) (2001) Cloning and Expression Vectors for Gene Function Analysis BioTechniques Press. Westborough, Mass. 298 pp. (ISBN 1-881299-21-X); Langer and Wise (eds.) (1974) Medical Applications of Controlled Release, CRC Pres., Boca Raton, Fla.; Old and Primrose (1985) Principles of Gene Manipulation: An Introduction To Genetic Engineering (3d Ed.) Blackwell Scientific Publications, Boston, Mass. Studies in Microbiology; V. 2:409 pp. (ISBN 0-632-01318-4); Sambrook et al. (eds.) (1989) Molecular Cloning: A Laboratory Manual (2d Ed.) Cold Spring Harbor Laboratory Press, NY, Vols. 1-3 (ISBN 0-87969-309-6); Robinson (ed.) (1978) Sustained and Controlled Release Drug Delivery Systems, Marcel Dekker, Inc., NY; Winnacker (1987) from Genes To Clones: Introduction To Gene Technology; VCH Publishers, NY (translated by Horst Ibelgaufts), 634 pp. (ISBN 0-89573-614-4).

### EQUIVALENTS

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

1. A method of treating a subject having rheumatoid arthritis (RA), wherein the subject is resistant to treatment with methotrexate, the method comprising administering a binding protein, wherein the binding protein specifically binds IL-17 and TNF-α and comprises three complementarity determining regions (CDRs) for binding TNF-α of a heavy chain variable region comprising the amino acid sequence of SEQ ID NO: 12, wherein
the CDR-H1 has an amino acid sequence NYGII;
the CDR-H2 has an amino acid sequence WINTYTGKPTYAQKFQ; and
the CDR-H3 has an amino acid sequence KLFTTMDVTDNAMDY;
and the three CDRs for binding IL-17 of a heavy chain variable region comprising the amino acid sequence of SEQ ID NO: 14, wherein
the CDR-H1 has an amino acid sequence DYEIH;
the CDR-H2 has an amino acid sequence VNDPESGGTFYNQ; and
the CDR-H3 has an amino acid sequence YSKWDSFDGMDY.

2. A method of treating a subject having rheumatoid arthritis (RA), wherein the subject is resistant to treatment with methotrexate, the method comprising administering a binding protein, wherein the binding protein specifically binds IL-17 and TNF-α and comprises three CDRs for binding TNF-α of a light chain variable region comprising the amino acid sequence of SEQ ID NO: 17, wherein
the CDR-L1 has an amino acid sequence RASQDISQYLN;
the CDR-L2 has an amino acid sequence YTSRLQS; and
the CDR-L3 has an amino acid sequence QQGNTWPPT;
and the three CDRs for binding IL-17 of a light chain variable region comprising the amino acid sequence of SEQ ID NO: 19, wherein
the CDR-L1 has an amino acid sequence RASSGIISYID;
the CDR-L2 has an amino acid sequence ATFDLAS; and
the CDR-L3 has an amino acid sequence RQVGSYPET.

3-5. (canceled)

6. The method of claim 1, wherein the binding protein comprises a heavy chain variable region comprising the amino acid sequence of SEQ ID NO: 11.

7. The method of claim 1, wherein the binding protein comprises a light chain variable region comprising the amino acid sequence of SEQ ID NO: 16.

8-36. (canceled)

37. A method for treating a subject having rheumatoid arthritis (RA), wherein the subject is resistant to treatment with methotrexate, the method comprising the step of administering to the subject a composition comprising a binding protein that specifically binds both IL-17 and TNF-α, wherein the binding protein is a dual variable domain immunoglobulin (DVD-Ig) protein, and wherein the binding protein comprises at least one variable heavy chain polypeptide comprising the amino acid sequence of SEQ ID NO: 11 and at least one variable light chain polypeptide comprising the amino acid sequence of SEQ ID NO: 16, wherein the binding protein is administered weekly and wherein the total amount of binding protein administered to the subject is about: 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, about 325-350 mg, or about 350-400 mg of the binding protein.

38. A method for treating a subject having rheumatoid arthritis (RA), wherein the subject has been or is currently being treated with methotrexate, the method comprising the step of administering to the subject a binding protein that specifically binds TNF-α and IL-17, wherein the binding protein is a dual variable domain immunoglobulin (DVD-Ig) binding protein, wherein the binding protein comprises a variable heavy chain comprising the amino acid sequence of SEQ ID NO: 11 and a variable light chain comprising the amino acid sequence of SEQ ID NO: 16, wherein administering the binding protein is performed using a dose of about: 0.005 (milligrams per kilogram) mg/kg to 0.01 mg/kg, 0.01 mg/kg to 0.05 mg/kg, 0.05 mg/kg to 0.1 mg/kg, 0.1 mg/kg to 0.5 mg/kg, 0.5 mg/kg to 1 mg/kg, 1 mg/kg to 1.5 mg/kg, 1.5 mg/kg to 2 mg/kg, 2 mg/kg to 3 mg/kg, 3 mg/kg to 4 mg/kg, 4 mg/kg to 5 mg/kg, 5 mg/kg to 6 mg/kg, 6 mg/kg to 7 mg/kg, 7 mg/kg to 8 mg/kg, 8 mg/kg to 9 mg/kg, or 9 mg/kg to 10 mg/kg of weight of the binding protein to weight of the subject.

39. A method for treating a subject having rheumatoid arthritis (RA), wherein the
subject has been or is currently being treated with methotrexate, the method comprising the step of administering to the subject a binding protein that specifically binds TNF-α and IL-17, wherein the binding protein is a dual variable domain immunoglobulin (DVD-Ig) binding protein, wherein the binding protein comprises a variable heavy chain comprising the amino acid sequence of SEQ ID NO: 11 and a variable light chain comprising the amino acid sequence of SEQ ID NO: 16, wherein administering the binding protein is performed using one dose or multiple doses of the binding protein.

40-44. (canceled)

45. The method of claim 39, wherein the improvement comprises using an improvement in a score, a test, or a metric for RA.

46. The method of claim 45, wherein the score, the test, or the metric is selected from the group consisting of: Physician Global Assessment of Disease Activity (Physician Global); Global Arthritis Score; a Patient Global Assessment of Disease Activity (PTGL); Patient Assessment of General Health (GH); Patient Assessment of General Health (GH); patient's assessment of pain; Patient Reported Outcome; global disease activity and physical function; a Health Assessment Questionnaire (HAQ-DI); measurement or presence of an anti-drug antibody (ADA); tender joint count (TJC); swollen joint count (SJC); Work Instability Scale for Rheumatoid Arthritis; Short Form Health Survey (SF-36); American College of Rheumatology (ACR) Criteria; ACR20; ACR50; ACR70; ACR Response Rate; proportion of subjects achieving Low Disease Activity (LDA); Disease Activity Score 28 (DAS28); DAS28 based on C-reactive protein; proportion of subjects achieving ACR70 responder status; Clinical Disease Activity Index (CDAI); simple disease activity index (SDAI); Clinical Remission criteria, and acute phase reactant levels.

47-53. (canceled)

54. The method of 39, wherein the subject has a resistance to a DMARD.

55-59. (canceled)

60. A dose of the binding protein of claim 1 that neutralizes TNF and IL-17 sufficient to treat or prevent at least one symptom of RA.

61. The dose of claim 60, wherein the dose comprises about 120 milligrams or about 240 milligrams.

62. A method for reducing a symptom of rheumatoid arthritis in a subject in need thereof, wherein the method comprises administering a binding protein comprising first and second polypeptide chains,
wherein the first polypeptide chain comprises a VD1-(X1)n-VD2-C-(X2)n, wherein;
VD1 is a first heavy chain variable domain;
VD2 is a second heavy chain variable domain;
C is a heavy chain constant domain;
X1 is a linker with the proviso that it is not CH1;
X2 is an Fc region; and
n is 0 or 1;
wherein the VD1 or VD2 heavy chain variable domain comprises three CDRs from the amino acid sequence of SEQ ID NO: 12; wherein the three CDRs comprise CDR-H1, CDR-H2, and CDR-H3, wherein each amino acid in the CDR-H1, the CDR-H2 and the CDR-H3 is represented by a one letter code; and wherein
the CDR-H1 has an amino acid sequence NYGII;
the CDR-H2 has an amino acid sequence WINTYTGKPTYAQKFQ; and
the CDR-H3 has an amino acid sequence KLFTTMDVTDNAMDY;
the other of the VD1 or VD2 heavy chain variable domain comprises three CDRs from the amino acid sequence of SEQ ID NO:14, wherein the CDRs comprise CDR-H1, CDR-H2, and CDR-H3, wherein
the CDR-H1 has an amino acid sequence DYEIH;
the CDR-H2 has an amino acid sequence VNDPESGGTFYNQ; and
the CDR-H3 has an amino acid sequence YSKWDSFDGMDY; and
wherein said second polypeptide chain comprises a second VD1-(X1)n-VD2-C-(X2)n, wherein
VD1 is a first light chain variable domain;
VD2 is a second light chain variable domain;
C is a light chain constant domain;
X1 is a linker with the proviso that it is not CH1;
X2 does not comprise an Fc region; and
n is 0 or 1;
the VD1 or VD2 light chain variable domain comprises three CDRs from the amino acid sequence of SEQ ID NO: 17, wherein the CDRs comprise CDR-L1, CDR-L2, and CDR-L3, wherein
the CDR-L1 has an amino acid sequence RASQDISQYLN;
the CDR-L2 has an amino acid sequence YTSRLQS; and
the CDR-L3 has an amino acid sequence QQGNTWPPT; and
the other of the VD1 or VD2 light chain variable domain comprises three SEQ ID NO:19, wherein the CDRs comprise CDR-L1, CDR-L2, and CDR-L3, wherein
the CDR-L1 has an amino acid sequence RASSGIISYID;
the CDR-L2 has an amino acid sequence ATFDLAS; and
the CDR-L3 has an amino acid sequence RQVGSYPET and
wherein the binding protein is capable of binding human IL-17 and TNF-α.

63. (canceled)

64. The method of claim 62, wherein n is 0 or 1 and X1 is a polypeptide comprising the amino acid sequence SEQ ID NO: 13.

65. The method of claim 62, wherein the heavy chain comprises the amino acid sequence SEQ ID NO: 11.

66. (canceled)

67. The method of claim 62, wherein n is 0 or 1 and the X1 of the first light chain variable domain comprises SEQ ID NO: 18.

68. The method of claim 62, wherein the light polypeptide chain comprises the amino acid sequence SEQ ID NO: 16.

69. The method of any of claim 62, wherein the binding protein comprises two heavy polypeptide chains and two light polypeptide chains.

70. The method of claim 68, wherein the binding protein further comprises a mutated Fc region.

71. The method of claim 70, wherein the Fc region comprises an amino acid sequence of SEQ ID NO: 15 or SEQ ID NO: 20.

72. The method of claim 62, wherein the subject having the rheumatoid arthritis is resistant to at least one disease-modifying antirheumatic drug (DMARD).

73. The method of claim 72, wherein the DMARD is methotrexate.

74. The method of any of claim 62, wherein the binding protein is administered subcutaneously or parenterally.

75. The method of claim 62, wherein the symptom is selected from the group consisting of inflammation, swelling, stiffness, pain, hyperplasia, and synovitis.

76. The binding protein of claim 62, wherein the VD1 of the first polypeptide chain comprises three CDRs, wherein the three CDRs are CDR-H1, CDR-H2 and CDR-H3, wherein CDR-H1 comprises the amino acid sequence of NYGII, CDR-H2 comprises the amino acid sequence of WINTYTGKPTYAQKFQ, and CDR-H3 comprises the amino acid sequence of KLFTTMDVTDNAMDY;
wherein the VD2 of the first polypeptide chain comprises three CDRs, wherein the three CDRs are CDR-H1, CDR-H2 and CDR-H3, wherein CDR-H1 comprises the amino acid sequence of DYEIH, CDR-H2 comprises the amino acid sequence of VNDPESGGTFYNQKFDG, and CDR-H3 comprises the amino acid sequence of YSKWDSFDGMDY;
wherein the VD1 of the second polypeptide chain comprises three CDRs, wherein the three CDRs are CDR-L1, CDR-L2 and CDR-L3, wherein CDR-L1 comprises the amino acid sequence of RASQDISQYLN, CDR-L2 comprises the amino acid sequence of YTSRLQS, and CDR-L3 comprises the amino acid sequence of QQGNTWPPTIS; and
wherein the VD2 of the second polypeptide chain comprises three CDRs, wherein the three CDRs are CDR-L1, CDR-L2 and CDR-L3, wherein CDR-L1 comprises the amino acid sequence of RASSGIISYID, CDR-L2 comprises the amino acid sequence of ATFDLAS, and CDR-L3 comprises the amino acid sequence of RQVGSYPET.

77. The binding protein of claim 62, wherein the VD1 of the first polypeptide chain comprises three CDRs, wherein the three CDRs are CDR-H1, CDR-H2 and CDR-H3, wherein CDR-H1 comprises the amino acid sequence of DYEIH, CDR-H2 comprises the amino acid sequence of VNDPESGGTFYNQKFDG, and CDR-H3 comprises the amino acid sequence of YSKWDSFDGMDY;
wherein the VD2 of the first polypeptide chain comprises three CDRs, wherein the three CDRs are CDR-H1, CDR-H2 and CDR-H3, wherein CDR-H1 comprises the amino acid sequence of NYGII, CDR-H2 comprises the amino acid sequence of WINTYTGKPTYAQKFQ, and CDR-H3 comprises the amino acid sequence of KLFTTMDVTDNAMDY;
wherein the VD1 of the second polypeptide chain comprises three CDRs, wherein the three CDRs are CDR-L1, CDR-L2 and CDR-L3, wherein CDR-L1 comprises the amino acid sequence of RASSGIISYID, CDR-L2 comprises the amino acid sequence of ATFDLAS, and CDR-L3 comprises the amino acid sequence of RQVGSYPET; and
wherein the VD2 of the second polypeptide chain comprises three CDRs, wherein the three CDRs are CDR-L1, CDR-L2 and CDR-L3, wherein CDR-L1 comprises the amino acid sequence of RASQDISQYLN, CDR-L2 comprises the amino acid sequence of YTSRLQS, and CDR-L3 comprises the amino acid sequence of QQGNTWPPTIS.


SEQUENCE LISTING (70 sequences)

SEQ ID NO: 1 - protein, 121 aa, Artificial Sequence
  Description of Artificial Sequence: Synthetic polypeptide
EFQLQQSGPELVKPGASVRISCKASGYSFTDYNMNWVKQSNGKSLEWVGVINPNYGSSTYNQKFKGKATLTVDQSSSTAYMQLNSLTSEDSAVYYCARKWGQLGRGFFDVWGTGTTVTVSS

SEQ ID NO: 2 - protein, 107 aa, Artificial Sequence
  Description of Artificial Sequence: Synthetic polypeptide
QIVLSQSPAILSASPGEKVTMTCRASSSVSYMHWFQQKPGSSPKPWIYATSNLASGVPARFSGSGSGTSYSLTISRVEAEDAATYYCQQWSSSPLTFGAGTKLELKR

SEQ ID NO: 3 - protein, 121 aa, Artificial Sequence
  Description of Artificial Sequence: Synthetic polypeptide
QVQLQQSGAELVRPGTSVTLSCKASGYIFTDYEIHWVKQTPVHGLEWIGVNDPESGGTFYNQKFDGKAELTADKSSSTAYMELRSLTSEDSGVYYCTRYYRYESFYGMDYWGQGTSITVSS

SEQ ID NO: 4 - protein, 107 aa, Artificial Sequence
  Description of Artificial Sequence: Synthetic polypeptide
QIVLTQSPAIMSASPGEKVTMTCSASSSISYIYWFQQKPGTSPKRWIYATFELASGVPARFSGSGSGTSYSLTISSMEAEDAATYYCHQRSSYPWTFGGGSKLEIKR

SEQ ID NO: 5 - protein, 252 aa, Artificial Sequence
  Description of Artificial Sequence: Synthetic polypeptide
EFQLQQSGPELVKPGASVRISCKASGYSFTDYNMNWVKQSNGKSLEWVGVINPNYGSSTYNQKFKGKATLTVDQSSSTAYMQLNSLTSEDSAVYYCARKWGQLGRGFFDVWGTGTTVTVSSGGGGSGGGGSQVQLQQSGAELVRPGTSVTLSCKASGYIFTDYEIHWVKQTPVHGLEWIGVNDPESGGTFYNQKFDGKAELTADKSSSTAYMELRSLTSEDSGVYYCTRYYRYESFYGMDYWGQGTSITVSS

SEQ ID NO: 6 - protein, 10 aa, Artificial Sequence
  Description of Artificial Sequence: Synthetic peptide
GGGGSGGGGS

SEQ ID NO: 7 - protein, 330 aa, Artificial Sequence
  Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 223 aa, Artificial Sequence
  Description of Artificial Sequence: Synthetic polypeptide
QIVLSQSPAILSASPGEKVTMTCRASSSVSYMHWFQQKPGSSPKPWIYATSNLASGVPARFSGSGSGTSYSLTISRVEAEDAATYYCQQWSSSPLTFGAGTKLELKRGGSGGGGSGQIVLTQSPAIMSASPGEKVTMTCSASSSISYIYWFQQKPGTSPKRWIYATFELASGVPARFSGSGSGTSYSLTISSMEAEDAATYYCHQRSSYPWTFGGGSKLEIKR

SEQ ID NO: 9 - protein, 9 aa, Artificial Sequence
  Description of Artificial Sequence: Synthetic peptide
GGSGGGGSG

SEQ ID NO: 10 - protein, 106 aa, Artificial Sequence
  Description of Artificial Sequence: Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 255 aa, Artificial Sequence
  Description of Artificial Sequence: Synthetic polypeptide
EVQLVQSGAEVKKPGASVKVSCKASGYTFANYGIIWVRQAPGQGLEWMGWINTYTGKPTYAQKFQGRVTMTTDTSTSTAYMELSSLRSEDTAVYYCARKLFTTMDVTDNAMDYWGQGTTVTVSSGGGGSGGGGSEVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 12 - protein, 124 aa, Artificial Sequence
  Description of Artificial Sequence: Synthetic polypeptide
EVQLVQSGAEVKKPGASVKVSCKASGYTFANYGIIWVRQAPGQGLEWMGWINTYTGKPTYAQKFQGRVTMTTDTSTSTAYMELSSLRSEDTAVYYCARKLFTTMDVTDNAMDYWGQGTTVTVSS

SEQ ID NO: 13 - protein, 10 aa, Artificial Sequence
  Description of Artificial Sequence: Synthetic peptide
GGGGSGGGGS

SEQ ID NO: 14 - protein, 121 aa, Artificial Sequence
  Description of Artificial Sequence: Synthetic polypeptide
EVQLVQSGAEVKKPGSSVKVSCKASGYTFTDYEIHWVRQAPGQGLEWMGVNDPESGGTFYNQKFDGRVTLTADESTSTAYMELSSLRSEDTAVYYCTRYSKWDSFDGMDYWGQGTTVTVSS

SEQ ID NO: 15 - protein, 330 aa, Artificial Sequence; identical to SEQ ID NO: 7
  Description of Artificial Sequence: Synthetic polypeptide

SEQ ID NO: 16 - protein, 225 aa, Artificial Sequence
  Description of Artificial Sequence: Synthetic polypeptide
DIQMTQSPSSLSASVGDRVTITCRASQDISQYLNWYQQKPGKAPKLLIYYTSRLQSGVPSRFSGSGSGTDFTLTISSLQPEDFATYFCQQGNTWPPTFGQGTKLEIKRGGSGGGGSGDIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 17 - protein, 108 aa, Artificial Sequence
  Description of Artificial Sequence: Synthetic polypeptide
DIQMTQSPSSLSASVGDRVTITCRASQDISQYLNWYQQKPGKAPKLLIYYTSRLQSGVPSRFSGSGSGTDFTLTISSLQPEDFATYFCQQGNTWPPTFGQGTKLEIKR

SEQ ID NO: 18 - protein, 9 aa, Artificial Sequence
  Description of Artificial Sequence: Synthetic peptide
GGSGGGGSG

SEQ ID NO: 19 - protein, 108 aa, Artificial Sequence
  Description of Artificial Sequence: Synthetic polypeptide
DIQMTQSPSSLSASVGDRVTITCRASSGIISYIDWFQQKPGKAPKRLIYATFDLASGVPSRFSGSGSGTDYTLTISSLQPEDFATYYCRQVGSYPETFGQGTKLEIKR

SEQ ID NO: 20 - protein, 106 aa, Artificial Sequence; identical to SEQ ID NO: 10
  Description of Artificial Sequence: Synthetic polypeptide

SEQ ID NO: 21-23 - skipped (3 entries)

SEQ ID NO: 24 - protein, 6 aa, Artificial Sequence
  Description of Artificial Sequence: Synthetic peptide
GGGGSG

SEQ ID NO: 25 - protein, 5 aa, Artificial Sequence
  Description of Artificial Sequence: Synthetic peptide
GGSGG

SEQ ID NO: 26 - protein, 10 aa, Artificial Sequence
  Description of Artificial Sequence: Synthetic peptide
GGGGSGGGGS

SEQ ID NO: 27 - protein, 9 aa, Artificial Sequence
  Description of Artificial Sequence: Synthetic peptide
GGSGGGGSG

SEQ ID NO: 28 - protein, 10 aa, Artificial Sequence
  Description of Artificial Sequence: Synthetic peptide
GGSGGGGSGS

SEQ ID NO: 29 - protein, 13 aa, Artificial Sequence
  Description of Artificial Sequence: Synthetic peptide
GGSGGGGSGGGGS

SEQ ID NO: 30 - protein, 14 aa, Artificial Sequence
  Description of Artificial Sequence: Synthetic peptide
GGGGSGGGGSGGGG

SEQ ID NO: 31 - protein, 15 aa, Artificial Sequence
  Description of Artificial Sequence: Synthetic peptide
GGGGSGGGGSGGGGS

SEQ ID NO: 32 - protein, 6 aa, Artificial Sequence
  Description of Artificial Sequence: Synthetic peptide
ASTKGP

SEQ ID NO: 33 - protein, 13 aa, Artificial Sequence
  Description of Artificial Sequence: Synthetic peptide
ASTKGPSVFPLAP

SEQ ID NO: 34 - protein, 5 aa, Artificial Sequence
  Description of Artificial Sequence: Synthetic peptide
TVAAP

SEQ ID NO: 35 - protein, 6 aa, Artificial Sequence
  Description of Artificial Sequence: Synthetic peptide
RTVAAP

SEQ ID NO: 36 - protein, 12 aa, Artificial Sequence
  Description of Artificial Sequence: Synthetic peptide
TVAAPSVFIFPP

SEQ ID NO: 37 - protein, 13 aa, Artificial Sequence
  Description of Artificial Sequence: Synthetic peptide
RTVAAPSVFIFPP

SEQ ID NO: 38 - protein, 16 aa, Artificial Sequence
  Description of Artificial Sequence: Synthetic peptide
AKTTPKLEEGEFSEAR

SEQ ID NO: 39 - protein, 17 aa, Artificial Sequence
  Description of Artificial Sequence: Synthetic peptide
AKTTPKLEEGEFSEARV

SEQ ID NO: 40 - protein, 9 aa, Artificial Sequence
  Description of Artificial Sequence: Synthetic peptide
AKTTPKLGG

SEQ ID NO: 41 - protein, 10 aa, Artificial Sequence
  Description of Artificial Sequence: Synthetic peptide
SAKTTPKLGG

SEQ ID NO: 42 - protein, 6 aa, Artificial Sequence
  Description of Artificial Sequence: Synthetic peptide
SAKTTP

SEQ ID NO: 43 - protein, 6 aa, Artificial Sequence
  Description of Artificial Sequence: Synthetic peptide
RADAAP

SEQ ID NO: 44 - protein, 9 aa, Artificial Sequence
  Description of Artificial Sequence: Synthetic peptide
RADAAPTVS

SEQ ID NO: 45 - protein, 12 aa, Artificial Sequence
  Description of Artificial Sequence: Synthetic peptide
RADAAAAGGPGS

SEQ ID NO: 46 - protein, 27 aa, Artificial Sequence
  Description of Artificial Sequence: Synthetic peptide
RADAAAAGGGGSGGGGSGGGGSGGGGS

SEQ ID NO: 47 - protein, 18 aa, Artificial Sequence
  Description of Artificial Sequence: Synthetic peptide
SAKTTPKLEEGEFSEARV

SEQ ID NO: 48 - protein, 5 aa, Artificial Sequence
  Description of Artificial Sequence: Synthetic peptide
ADAAP

SEQ ID NO: 49 - protein, 12 aa, Artificial Sequence
  Description of Artificial Sequence: Synthetic peptide
ADAAPTVSIFPP

SEQ ID NO: 50 - protein, 6 aa, Artificial Sequence
  Description of Artificial Sequence: Synthetic peptide
QPKAAP

SEQ ID NO: 51 - protein, 13 aa, Artificial Sequence
  Description of Artificial Sequence: Synthetic peptide
QPKAAPSVTLFPP

SEQ ID NO: 52 - protein, 6 aa, Artificial Sequence
  Description of Artificial Sequence: Synthetic peptide
AKTTPP

SEQ ID NO: 53 - protein, 13 aa, Artificial Sequence
  Description of Artificial Sequence: Synthetic peptide
AKTTPPSVTPLAP

SEQ ID NO: 54 - protein, 6 aa, Artificial Sequence
  Description of Artificial Sequence: Synthetic peptide
AKTTAP

SEQ ID NO: 55 - protein, 13 aa, Artificial Sequence
  Description of Artificial Sequence: Synthetic peptide
AKTTAPSVYPLAP

SEQ ID NO: 56 - protein, 15 aa, Artificial Sequence
  Description of Artificial Sequence: Synthetic peptide
GENKVEYAPALMALS

SEQ ID NO: 57 - protein, 15 aa, Artificial Sequence
  Description of Artificial Sequence: Synthetic peptide
GPAKELTPLKEAKVS

SEQ ID NO: 58 - protein, 15 aa, Artificial Sequence
  Description of Artificial Sequence: Synthetic peptide
GHEAAAVMQVQYPAS

SEQ ID NO: 59 - skipped

SEQ ID NO: 60 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 330 aa, Artificial Sequence; identical to SEQ ID NO: 60
  Description of Artificial Sequence: Synthetic polypeptide

SEQ ID NO: 62 - protein, 106 aa, Artificial Sequence; identical to SEQ ID NO: 10
  Description of Artificial Sequence: Synthetic polypeptide

SEQ ID NO: 63 - protein, 104 aa, Artificial Sequence
  Description of Artificial Sequence: Synthetic polypeptide
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 64 - protein, 132 aa, Homo sapiens
GITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 65 - protein, 133 aa, Homo sapiens [sequence omitted]

SEQ ID NO: 66 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 67 - protein, 4 aa, Artificial Sequence
  Description of Artificial Sequence: Synthetic peptide
  MOD_RES (3)..(3): Any amino acid
FGXG

SEQ ID NO: 68 - protein, 9 aa, Artificial Sequence
  Description of Artificial Sequence: Synthetic peptide
  MOD_RES (2)..(9): Any amino acid
CXXXXXXXX

SEQ ID NO: 69 - protein, 5 aa, Artificial Sequence
  Description of Artificial Sequence: Synthetic peptide
LEWIG

SEQ ID NO: 70 - protein, 4 aa, Artificial Sequence
  Description of Artificial Sequence: Synthetic peptide
  MOD_RES (3)..(3): Any amino acid
WGXG